SEQUENCE LISTING (57 sequences)

SEQ ID NO: 1 - protein, 541 aa, Artificial Sequence
  EGFR
MRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 2 - protein, 410 aa, Artificial Sequence
  IGF1R
MHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 3 - protein, 433 aa, Artificial Sequence
  KIT
MYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 4 - protein, 555 aa, Artificial Sequence
  VEGFR1
MKRSSSEIKTDYLSIIMDPDEVPLDEQCERLPYDASKWEFARERLKLGKSLGRGAFGKVVQASAFGIKKSPTCRTVAVKMLKEGATASEYKALMTELKILHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 5 - protein, 424 aa, Artificial Sequence
  FGFR1
MKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 6 - protein, 355 aa, Artificial Sequence
  TRKA
MGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 7 - protein, 435 aa, Artificial Sequence
  MET
MWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIMKDFSHPNVLSLLGICLRSEGSPLVVLPYMKHGDLRNFIRNETHNPTVKDLIGFGLQVAGMKYLASKKFVHRDLAARNCMLDEKFTVKVADFGLARDMYDKEYYSVHNKTGAKLPVWMALESLQTQKFTTKSDVWSFGVVLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 8 - protein, 422 aa, Artificial Sequence
  EPHB4
MSNGREAEYSDKHGQYLIGHGTKVYIDPFTYEDPNEAVREFAKEIDVSYVKIEEVIGAGEFGEVCRGRLKAPGKKESCVAIKTLKGGYTERQRREFLSEASIMGQFEHPNIIRLEGVVTNSMPVMILTEFMENGALDSFLRLNDGQFTVIQLVGMLRGIASGMRYLAEMSYVHRDLAARNILVNSNLVCKVSDFGLSRFLEENSSDPTYTSSLGGKIPIRWTAPEAIAFRKFTSASDAWSYGIVMWEVMSFGERPYWDMSNQDVINAIEQDYRLPPPPDCPTSLHQLMLDCWQKDRNARPRFPQVVSALDKMIRNPASLKIVARENGGASHPLLDQRQPHYSAFGSVGEWLRAIKMGRYEESFAAAGFGSFELVSQISAEDLLRIGVTLAGHQKKILASVQHMKSQAKPGTPGGTGGPAPQY

SEQ ID NO: 9 - protein, 419 aa, Artificial Sequence
  AXL
MKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGGQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 10 - protein, 352 aa, Artificial Sequence
  TIE1
MRSCLHRRRTFTYQSGSGEETILQFSSGTLTLTRRPKLQPEPLSYPVLEWEDITFEDLIGEGNFGQVIRAMIKKDGLKMNAAIKMLKEYASENDHRDFAGELEVLCKLGHHPNIINLLGACKNRGYLYIAIEYAPYGNLLDFLRKSRVLETDPAFAREHGTASTLSSRQLLRFASDAANGMQYLSEKQFIHRDLAARNVLVGENLASKIADFGLSRGEEVYVKKTMGRLPVRWMAIESLNYSVYTTKSDVWSFGVLLWEIVSLGGTPYCGMTCAELYEKLPQGYRMEQPRNCDDEVYELMRQCWRDRPYERPPFAQIALQLGRMLEARKAYVNMSLFENFTYAGIDATAEEA

SEQ ID NO: 11 - protein, 474 aa, Artificial Sequence
  DDR1
MLHWRRLLSKAERRVLEEELTVHLSVPGDTILINNRPGPREPPPYQEPRPRGNPPHSAPCVPNGSALLLSNPAYRLLLATYARPPRGPGPPTPAWAKPTNTQAYSGDYMEPEKPGAPLLPPPPQNSVPHYAEADIVTLQGVTGGNTYAVPALPPGAVGDGPPRVDFPRSRLRFKEKLGEGQFGEVHLCEVDSPQDLVSLDFPLNVRKGHPLLVAVKILRPDATKNARNDFLKEVKIMSRLKDPNIIRLLGVCVQDDPLCMITDYMENGDLNQFLSAHQLEDKAAEGAPGDGQAAQGPTISYPMLLHVAAQIASGMRYLATLNFVHRDLATRNCLVGENFTIKIADFGMSRNLYAGDYYRVQGRAVLPIRWMAWECILMGKFTTASDVWAFGVTLWEVLMLCRAQPFGQLTDEQVIENAGEFFRDQGRQVYLSRPPACPQGLYELMLRCWSRESEQRPPFSQLHRFLAEDALNTV

SEQ ID NO: 12 - protein, 454 aa, Artificial Sequence
  RET
MKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 13 - protein, 617 aa, Artificial Sequence
  ROS
MVYKTGENSTSLPESFKTKAGVPNKPGIPKLLEGSKNSIQWEKAEDNGCRITYYILEIRKSTSNNLQNQNLRWKMTFNGSCSSVCTWKSKNLKGIFQFRVVAANNLGFGEYSGISENIILVGDDFWIPETSFILTIIVGIFLVVTIPLTFVWHRRLKNQKSAKEGVTVLINEDKELAELRGLAAGVGLANACYAIHTLPTQEEIENLPAFPREKLTLRLLLGSGAFGEVYEGTAVDILGVGSGEIKVAVKTLKKGSTDQEKIEFLKEAHLMSKFNHPNILKQLGVCLLNEPQYIILELMEGGDLLTYLRKARMATFYGPLLTLVDLVDLCVDISKGCVYLERMHFIHRDLAARNCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQNQLQLFRNFFLNSIYQCRDEANNSGVINESFEGEDGDVICLNSDDIMPVVLMETKNREGLNYMVLATECGQGEEKSEGPLGSQESESCGLRKEEKEPHADKDFCQEKQVAYCPSGKPEGLNYACLTHSGYGDGSD

SEQ ID NO: 14 - protein, 296 aa, Artificial Sequence
  ALK
MTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIE

SEQ ID NO: 15 - protein, 517 aa, Artificial Sequence
  ROR1
MFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWMEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 16 - protein, 359 aa, Artificial Sequence
  MUSK
MITTLYCCRRRKQWKNKKRESAAVTLTTLPSELLLDRLHPNPMYQRMPLLLNPKLLSLEYPRNNIEYVRDIGEGAFGRVFQARAPGLLPYEPFTMVAVKMLKEEASADMQADFQREAALMAEFDNPNIVKLLGVCAVGKPMCLLFEYMAYGDLNEFLRSMSPHTVCSLSHSDLSMRAQVSSPGPPPLSCAEQLCIARQVAAGMAYLSERKFVHRDLATRNCLVGENMVVKIADFGLSRNIYSADYYKANENDAIPIRWMPPESIFYNRYTTESDVWAYGVVLWEIFSYGLQPYYGMAHEEVIYYVRDGNILSCPENCPVELYNLMRLCWSKLPADRPSFTSIHRILERMCERAEGTVSV

SEQ ID NO: 17 - protein, 536 aa, Artificial Sequence
  SRC
MGSNKSKPKDASQRRRSLEPAENVHGAGGGAFPASQTPSKPASADGHRGPSAAFAPAAAEPKLFGGFNSSDTVTSPQRAGPLAGGVTTFVALYDYESRTETDLSFKKGERLQIVNNTEGDWWLAHSLSTGQTGYIPSNYVAPSDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL

SEQ ID NO: 18 - protein, 600 aa, Artificial Sequence
  ABL
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLF

SEQ ID NO: 19 - protein, 763 aa, Artificial Sequence
  JAK1
MELKLSSHEEALSFVSLVDGYFRLTADAHHYLCTDVAPPLIVHNIQNGCHGPICTEYAINKLRQEGSEEGMYVLRWSCTDFDNILMTVTCFEKSEQVQGAQKQFKNFQIEVQKGRYSLHGSDRSFPSLGDLMSHLKKQILRTDNISFMLKRCCQPKPREISNLLVATKKAQEWQPVYPMSQLSFDRILKKDLVQGEHLGRGTRTHIYSGTLMDYKDDEGTSEEKKIKVILKVLDPSHRDISLAFFEAASMMRQVSHKHIVYLYGVCVRDVENIMVEEFVEGGPLDLFMHRKSDVLTTPWKFKVAKQLASALSYLEDKDLVHGNVCTKNLLLAREGIDSECGPFIKLSDPGIPITVLSRQECIERIPWIAPECVEDSKNLSVAADKWSFGTTLWEICYNGEIPLKDKTLIEKERFYESRCRPVTPSCKELADLMTRCMNYDPNQRPFFRAIMRDINKLEEQNPDIVSRKKNQPTEVDPTHFEKRFLKRIRDLGEGHFGKVELCRYDPEDNTGEQVAVKSLKPESGGNHIADLKKEIEILRNLYHENIVKYKGICTEDGGNGIKLIMEFLPSGSLKEYLPKNKNKINLKQQLKYAVQICKGMDYLGSRQYVHRDLAARNVLVESEHQVKIGDFGLTKAIETDKEYYTVKDDRDSPVFWYAPECLMQSKFYIASDVWSFGVTLHELLTYCDSDSSPMALFLKMIGPTHGQMTVTRLVNTLKEGKRLPCPPNCPDEVYQLMRKCWEFQPSNRTSFQNLIEGFEALLK

SEQ ID NO: 20 - protein, 531 aa, Artificial Sequence
  ACK1
MQPEEGTGWLLELLSEVQLQQYFLRLRDDLNVTRLSHFEYVKNEDLEKIGMGRPGQRRLWEAVKRRKALCKRKSWMSKVFSGKRLEAEFPPHHSQSTFRKTSPAPGGPAGEGPLQSLTCLIGEKDLRLLEKLGDGSFGVVRRGEWDAPSGKTVSVAVKCLKPDVLSQPEAMDDFIREVNAMHSLDHRNLIRLYGVVLTPPMKMVTELAPLGSLLDRLRKHQGHFLLGTLSRYAVQVAEGMGYLESKRFIHRDLAARNLLLATRDLVKIGDFGLMRALPQNDDHYVMQEHRKVPFAWCAPESLKTRTFSHASDTWMFGVTLWEMFTYGQEPWIGLNGSQILHKIDKEGERLPRPEDCPQDIYNVMVQCWAHKPEDRPTFVALRDFLLEAQPTDMRALQDFEEPDKLHIQMNDVITVIEGRAENYWWRGQNTRTLCVGPFPRNVVTSVAGLSAQDISQPLQNSFIHTGHGDSDPRHCWGFPDRIDELYLGNPMDPPDLLSVELSTSRPPQHLGGVKKPTYDPVSEDQDPLSSD

SEQ ID NO: 21 - protein, 401 aa, Artificial Sequence
  FAK
MVNGTSQSFIIRPQKEGERALPSIPKLANSEKQGMRTHAVSVSETDDYAEIIDEEDTYTMPSTRDYEIQRERIELGRCIGEGQFGDVHQGIYMSPENPALAVAIKTCKNCTSDSVREKFLQEALTMRQFDHPHIVKLIGVITENPVWIIMELCTLGELRSFLQVRKYSLDLASLILYAYQLSTALAYLESKRFVHRDIAARNVLVSSNDCVKLGDFGLSRYMEDSTYYKASKGKLPIKWMAPESINFRRFTSASDVWMFGVCMWEILMHGVKPFQGVKNNDVIGRIENGERLPMPPNCPPTLYSLMTKCWAYDPSRRPRFTELKAQLSTILEEEKAQQEERMRMESRRQATVSWDSGGSDEAPPKPSRPGYPSPRSSEGFYPSPQHMVQTNHYQVSGYPGS

SEQ ID NO: 22 - protein, 423 aa, Artificial Sequence
  FES
MLLQDDRHSTSSSEQEREGGRTPTLEILKSHISGIFRPKFSLPPPLQLIPEVQKPLHEQLWYHGAIPRAEVAELLVHSGDFLVRESQGKQEYVLSVLWDGLPRHFIIQSLDNLYRLEGEGFPSIPLLIDHLLSTQQPLTKKSGVVLHRAVPKDKWVLNHEDLVLGEQIGRGNFGEVFSGRLRADNTLVAVKSCRETLPPDLKAKFLQEARILKQYSHPNIVRLIGVCTQKQPIYIVMELVQGGDFLTFLRTEGARLRVKTLLQMVGDAAAGMEYLESKCCIHRDLAARNCLVTEKNVLKISDFGMSREEADGVYAASGGSRQVPVKWTAPEALNYGRYSSESDVWSFGILLWETFSLGASPYPNLSNQQTREFVEKGGRLPCPELCPDAVFRLMEQCWAYEPGQRPSFSTIYQELQSIRKRHR

SEQ ID NO: 23 - protein, 451 aa, Artificial Sequence
  BRK
MVSRDQAHLGPKYVGLWDFKSRTDEELSFRAGDVFHVARKEEQWWWATLLDEAGGAVAQGYVPHNYLAERETVESEPWFFGCISRSEAVRRLQAEGNATGAFLIRVSEKPSADYVLSVRDTQAVRHYKIWRRAGGRLHLNEAVSFLSLPELVNYHRAQSLSHGLRLAAPCRKHEPEPLPHWDDWERPREEFTLCRKLGSGYFGEVFEGLWKDRVQVAIKVISRDNLLHQQMLQSEIQAMKKLRHKHILALYAVVSVGDPVYIITELMAKGSLLELLRDSDEKVLPVSELLDIAWQVAEGMCYLESQNYIHRDLAARNILVGENTLCKVGDFGLARLIKEDVYLSHDHNIPYKWTAPEALSRGHYSTKSDVWSFGILLHEMFSRGQVPYPGMSNHEAFLRVDAGYRMPCPLECPPSVHKLMLTCWCRDPEQRPCFKALRERLSSFTSYENPT

SEQ ID NO: 24 - protein, 481 aa, Artificial Sequence
  TEC
MSIRKALPPAPETKKRRPPPPIPLEEEDNSEEIVVAMYDFQAAEGHDLRLERGQEYLILEKNDVHWWRARDKYGNEGYIPSNYVTGKKSNNLDQYEWYCRNMNRSKAEQLLRSEDKEGGFMVRDSSQPGLYTVSLYTKFGGEGSSGFRHYHIKETTTSPKKYYLAEKHAFGSIPEIIEYHKHNAAGLVTRLRYPVSVKGKNAPTTAGFSYEKWEINPSELTFMRELGSGLFGVVRLGKWRAQYKVAIKAIREGAMCEEDFIEEAKVMMKLTHPKLVQLYGVCTQQKPIYIVTEFMERGCLLNFLRQRQGHFSRDVLLSMCQDVCEGMEYLERNSFIHRDLAARNCLVSEAGVVKVSDFGMARYFLDDQYTSSSGAKFPVKWCPPEVFNYSRFSSKSDVWSFGVLMWEVFTEGRMPFEKYTNYEVVTMVTRGHRLYQPKLASNYVYEVMLRCWQEKPEGRPSFEDLLRTIDELVECEETFGR

SEQ ID NO: 25 - protein, 619 aa, Artificial Sequence
  ZAP70
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 26 - protein, 507 aa, Artificial Sequence
  BLK
GSGLVSSKKPDKEKPIKEKDKGQWSPLKVSAQDKDAPPLPPLVVFNHLTPPPPDEHLDEDKHFVVALYDYTAMNDRDLQMLKGEKLQVLKGTGDWWLARSLVTGREGYVPSNFVARVESLEMERWFFRSQGRKEAERQLLAPINKAGSFLIRESETNKGAFSLSVKDVTTQGELIKHYKIRCLDEGGYYISPRITFPSLQALVQHYSKKGDGLCQRLTLPCVRPAPQNPWAQDEWEIPRQSLRLVRKLGSGQFGEVWMGYYKNNMKVAIKTLKEGTMSPEAFLGEANVMKALQHERLVRLYAVVTKEPIYIVTEYMARGCLLDFLKTDEGSRLSLPRLIDMSAQIAEGMAYIERMNSIHRDLRAANILVSEALCCKIADFGLARIIDSEYTAQEGAKFPIKWTAPEAIHFGVFTIKADVWSFGVLLMEVVTYGRVPYPGMSNPEVIRNLERGYRMPRPDTCPPELYRGVIAECWRSRPEERPTFEFLQSVLEDFYTATERQYELQPS

SEQ ID NO: 27 - protein, 677 aa, Artificial Sequence
  BMX
RSDTKSILEELLLKRSQQKKKMSPNNYKERLFVLTKTNLSYYEYDKMKRGSRKGSIEIKKIRCVEKVNLEEQTPVERQYPFQIVYKDGLLYVYASNEESRSQWLKALQKEIRGNPHLLVKYHSGFFVDGKFLCCQQSCKAAPGCTLWEAYANLHTAVNEEKHRVPTFPDRVLKIPRAVPVLKMDAPSSSTTLAQYDNESKKNYGSQPPSSSTSLAQYDSNSKKIYGSQPNFNMQYIPREDFPDWWQVRKLKSSSSSEDVASSNQKERNVNHTTSKISWEFPESSSSEEEENLDDYDWFAGNISRSQSEQLLRQKGKEGAFMVRNSSQVGMYTVSLFSKAVNDKKGTVKHYHVHTNAENKLYLAENYCFDSIPKLIHYHQHNSAGMITRLRHPVSTKANKVPDSVSLGNGIWELKREEITLLKELGSGQFGVVQLGKWKGQYDVAVKMIKEGSMSEDEFFQEAQTMMKLSHPKLVKFYGVCSKEYPIYIVTEYISNGCLLNYLRSHGKGLEPSQLLEMCYDVCEGMAFLESHQFIHRDLAARNCLVDRDLCVKVSDFGMTRYVLDDQYVSSVGTKFPVKWSAPEVFHYFKYSSKSDVWAFGILMWEVFSLGKQPYDLYDNSQVVLKVSQGHRLYRPHLASDTIYQIMYSCWHELPEKRPTFQQLLSSIEPLREKDKQS

SEQ ID NO: 28 - protein, 655 aa, Artificial Sequence
  BTK
GSILESIFLKRSQQKKKTSPLNFKKRLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIRYNSDLVQKYHPCFWIDGQYLCCSQTAKNAMGCQILENRNGSLKPGSSHRKTKKPLPPTPEEDQILKKPLPPEPAAAPVSTSELKKVVALYDYMPMNANDLQLRKGDEYFILEESNLPWWRARDKNGQEGYIPSNYVTEAEDSIEMYEWYSKHMTRSQAEQLLKQEGKEGGFIVRDSSKAGKYTVSVFAKSTGDPQGVIRHYVVCSTPQSQYYLAEKHLFSTIPELINYHQHNSAGLISRLKYPVSQQNKNAPSTAGLGYGSWEIDPKDLTFLKELGTGQFGVVKYGKWRGQYDVAIKMIKEGSMSEDEFIEEAKVMMNLSHEKLVQLYGVCTKQRPIFIITEYMANGCLLNYLREMRHRFQTQQLLEMCKDVCEAMEYLESKQFLHRDLAARNCLVNDQGVVKVSDFGLSRYVLDDEYTSSVGSKFPVRWSPPEVLMYSKFSSKSDIWAFGVLMWEIYSLGKMPYERFTNSETAEHIAQGLRLYRPHLASEKVYTIMYSCWHEKADERPTFKILLSNILDVMDS

SEQ ID NO: 29 - protein, 431 aa, Artificial Sequence
  CSFR
GSKYQVRWKIIESYEGNSYTFIDPTQLPYNEKWEFPRNNLQFGKTLGAGAFGKVVEATAFGLGKEDAVLKVAVKMLKSTAHADEKESLMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFCS

SEQ ID NO: 30 - protein, 452 aa, Artificial Sequence
  CSK
GSSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHLS

SEQ ID NO: 31 - protein, 509 aa, Artificial Sequence
  CTK
GSAGRGSLVSWRAFHGCDSAEELPRVSPRFLRAWHPPPVSARMPTRRWAPGTQCITKCEHTRPKPGELAFRKGDVVTILEACENKSWYRVKHHTSGQEGLLAAGALREREALSADPKLSLMPWFHGKISGQEAVQQLQPPEDGLFLVRESARHPGDYVLCVSFGRDVIHYRVLHRDGHLTIDEAVFFCNLMDMVEHYSKDKGAICTKLVRPKRKHGTKSAEEELARAGWLLNLQHLTLGAQIGEGEFGAVLQGEYLGQKVAVKNIKCDVTAQAFLDETAVMTKMQHENLVRLLGVILHQGLYIVMEHVSKGNLVNFLRTRGRALVNTAQLLQFSLHVAEGMEYLESKKLVHRDLAARNILVSEDLVAKVSDFGLAKAERKGLDSSRLPVKWTAPEALKHGKFTSKSDVWSFGVLLWEVFSYGRAPYPKMSLKEVSEAVEKGYRMEPPEGCPGPVHVLMSSCWEAEPARRPPFRKLAEKLARELRSAGAPASVSGQDADGSTSPRSQEPS

SEQ ID NO: 32 - protein, 466 aa, Artificial Sequence
  DDR2
GSVDDSNTRILIGCLVAIIFILLAIIVIILWRQFWQKMLEKASRRMLDDEMTVSLSLPSDSSMFNNNRSSSPSEQGSNSTYDRIFPLRPDYQEPSRLIRKLPEFAPGEEESGCSGVVKPVQPSGPEGVPHYAEADIVNLQGVTGGNTYSVPAVTMDLLSGKDVAVEEFPRKLLTFKEKLGEGQFGEVHLCEVEGMEKFKDKDFALDVSANQPVLVAVKMLRADANKNARNDFLKEIKIMSRLKDPNIIHLLAVCITDDPLCMITEYMENGDLNQFLSRHEPPNSSSSDVRTVSYTNLKFMATQIASGMKYLSSLNFVHRDLATRNCLVGKNYTIKIADFGMSRNLYSGDYYRIQGRAVLPIRWMSWESILLGKFTTASDVWAFGVTLWETFTFCQEQPYSQLSDEQVIENTGEFFRDQGRQTYLPQPAICPDSVYKLMLSCWRRDTKNRPSFQEIHLLLLQQGDDS

SEQ ID NO: 33 - protein, 419 aa, Artificial Sequence
  EPHA2
RSRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPIS

SEQ ID NO: 34 - protein, 379 aa, Artificial Sequence
  EPHA4
GSSKYSKAKQEADEEKHLNQGVRTYVDPFTYEDPNQAVREFAKEIDASCIKIEKVIGVGEFGEVCSGRLKVPGKREICVAIKTLKAGYTDKQRRDFLSEASIMGQFDHPNIIHLEGVVTKCKPVMIITEYMENGSLDAFLRKNDGRFTVIQLVGMLRGIGSGMKYLSDMSYVHRDLAARNILVNSNLVCKVSDFGMSRVLEDDPEAAYTTRGGKIPIRWTAPEAIAYRKFTSASDVWSYGIVMWEVMSYGERPYWDMSNQDVIKAIEEGYRLPPPMDCPIALHQLMLDCWQKERSDRPKFGQIVNMLDKLIRNPNSLKRTGTESSRPNTALLDPSSPEFSAVVSVGDWLQAIKMDRYKDNFTAAGYTTLEAVVHVNQDS

SEQ ID NO: 35 - protein, 424 aa, Artificial Sequence
  FGFR2
GSKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKTS

SEQ ID NO: 36 - protein, 415 aa, Artificial Sequence
  FGFR4
GSRGQALHGRHPRPPATVQKLSRFPLARQFSLESGSSGKSSSSLVRGVRLSSSGPALLAGLVSLDLPLDPLWEFPRDRLVLGKPLGEGCFGQVVRAEAFGMDPARPDQASTVAVKMLKDNASDKDLADLVSEMEVMKLIGRHKNIINLLGVCTQEGPLYVIVECAAKGNLREFLRARRPPGPDLSPDGPRSSEGPLSFPVLVSCAYQVARGMQYLESRKCIHRDLAARNVLVTEDNVMKIADFGLARGVHHIDYYKKTSNGRLPVKWMAPEALFDRVYTHQSDVWSFGILLWEIFTLGGSPYPGIPVEELFSLLREGHRMDRPPHCPPELYGLMRECWHAAPSQRPTFKQLVEALDKVLLAVSEEYLDLRLTFGPYSPSGGDASSTCSSSDSVFSHDPLPLGSSSFPFGSGVQTS

SEQ ID NO: 37 - protein, 532 aa, Artificial Sequence
  FGR
RSGCVFCKKLEPVATAKEDAGLEGDFRSYGAADHYGPDPTKARPASSFAHIPNYSNFSSQAINPGFLDSGTIRGVSGIGVTLFIALYDYEARTEDDLTFTKGEKFHILNNTEGDWWEARSLSSGKTGCIPSNYVAPVDSIQAEEWYFGKIGRKDAERQLLSPGNPQGAFLIRESETTKGAYSLSIRDWDQTRGDHVKHYKIRKLDMGGYYITTRVQFNSVQELVQHYMEVNDGLCNLLIAPCTIMKPQTLGLAKDAWEISRSSITLERRLGTGCFGDVWLGTWNGSTKVAVKTLKPGTMSPKAFLEEAQVMKLLRHDKLVQLYAVVSEEPIYIVTEFMCHGSLLDFLKNPEGQDLRLPQLVDMAAQVAEGMAYMERMNYIHRDLRAANILVGERLACKIADFGLARLIKDDEYNPCQGSKFPIKWTAPEAALFGRFTIKSDVWSFGILLTELITKGRIPYPGMNKREVLEQVEQGYHMPCPPGCPASLYEAMEQTWRLDPEERPTFEYLQSFLEDYFTSAEPQYQPGDQTYS

SEQ ID NO: 38 - protein, 423 aa, Artificial Sequence
  FLT3
GSRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVS

SEQ ID NO: 39 - protein, 507 aa, Artificial Sequence
  FRK
GSSNICQRLWEYLEPYLPCLSTEADKSTVIENPGALCSPQSQRHGHYFVALFDYQARTAEDLSFRAGDKLQVLDTLHEGWWFARHLEKRRDGSSQQLQGYIPSNYVAEDRSLQAEPWFFGAIGRSDAEKQLLYSENKTGSFLIRESESQKGEFSLSVLDGAVVKHYRIKRLDEGGFFLTRRRIFSTLNEFVSHYTKTSDGLCVKLGKPCLKIQVPAPFDLSYKTVDQWEIDRNSIQLLKRLGSGQFGEVWEGLWNNTTPVAVKTLKPGSMDPNDFLREAQIMKNLRHPKLIQLYAVCTLEDPIYIITELMRHGSLQEYLQNDTGSKIHLTQQVDMAAQVASGMAYLESRNYIHRDLAARNVLVGEHNIYKVADFGLARVFKVDNEDIYESRHEIKLPVKWTAPEAIRSNKFSIKSDVWSFGILLYEIITYGKMPYSGMTGAQVIQMLAQNYRLPQPSNCPQQFYNIMLECWNAEPKERPTFETLRWKLEDYFETDSSYSDANNFISS

SEQ ID NO: 40 - protein, 540 aa, Artificial Sequence
  FYN
GSMGCVQCKDKEATKLTEERDGSLNQSSGYRYGTDPTPQHYPSFGVTSIPNYNNFHAAGGQGLTVFGGVNSSSHTGTLRTRGGTGVTLFVALYDYEARTEDDLSFHKGEKFQILNSSEGDWWEARSLTTGETGYIPSNYVAPVDSIQAEEWYFGKLGRKDAERQLLSFGNPRGTFLIRESETTKGAYSLSIRDWDDMKGDHVKHYKIRKLDNGGYYITTRAQFETLQQLVQHYSERAAGLCCRLVVPCHKGMPRLTDLSVKTKDVWEIPRESLQLIKRLGNGQFGEVWMGTWNGNTKVAIKTLKPGTMSPESFLEEAQIMKKLKHDKLVQLYAVVSEEPIYIVTEYMNKGSLLDFLKDGEGRALKLPNLVDMAAQVAAGMAYIERMNYIHRDLRSANILVGNGLICKIADFGLARLIEDNEYTARQGAKFPIKWTAPEAALYGRFTIKSDVWSFGILLTELVTKGRVPYPGMNNREVLEQVERGYRMPCPQDCPISLHELMIHCWKKDPEERPTFEYLQSFLEDYFTATEPQYQPGENLS

SEQ ID NO: 41 - protein, 609 aa, Artificial Sequence
  HER2
GSSIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAS

SEQ ID NO: 42 - protein, 615 aa, Artificial Sequence
  HER3
GSEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRTS

SEQ ID NO: 43 - protein, 634 aa, Artificial Sequence
  JAK2
GSKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCFCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAGS

SEQ ID NO: 44 - protein, 639 aa, Artificial Sequence
  JAK3
RSQRGHSPPTSSLVQPQSQYQLSQMTFHKIPADSLEWHENLGHGSFTKIYRGCRHEVVDGEARKTEVLLKVMDAKHKNCMESFLEAASLMSQVSYRHLVLLHGVCMAGDSTMVQEFVHLGAIDMYLRKRGHLVPASWKLQVVKQLAYALNYLEDKGLPHGNVSARKVLLAREGADGSPPFIKLSDPGVSPAVLSLEMLTDRIPWVAPECLREAQTLSLEADKWGFGATVWEVFSGVTMPISALDPAKKLQFYEDRQQLPAPKWTELALLIQQCMAYEPVQRPSFRAVIRDLNSLISSDYELLSDPTPGALAPRDGLWNGAQLYACQDPTIFEERHLKYISQLGKGNFGSVELCRYDPLAHNTGALVAVKQLQHSGPDQQRDFQREIQILKALHSDFIVKYRGVSYGPGRPELRLVMEYLPSGCLRDFLQRHRARLDASRLLLYSSQICKGMEYLGSRRCVHRDLAARNILVESEAHVKIADFGLAKLLPLDKDYYVVREPGQSPIFWYAPESLSDNIFSRQSDVWSFGVVLYELFTYCDKSCSPSAEFLRMMGCERDVPALCRLLELLEEGQRLPAPPACPAEVHELMKLCWAPSPQDRPSFSALGPQLDMLWSGSRGCETHAFTAHPEGKHHSLSFSS

SEQ ID NO: 45 - protein, 551 aa, Artificial Sequence
  KDR
GSDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPVS

SEQ ID NO: 46 - protein, 498 aa, Artificial Sequence
  LCK
GSENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQPS

SEQ ID NO: 47 - protein, 514 aa, Artificial Sequence
  LYN
GSGCIKSKGKDSLSDDGVDLKTQPVRNTERTIYVRDPTSNKQQRPVPESQLLPGQRFQTKDPEEQGDIVVALYPYDGIHPDDLSFKKGEKMKVLEEHGEWWKAKSLLTKKEGFIPSNYVAKLNTLETEEWFFKDITRKDAERQLLAPGNSAGAFLIRESETLKGSFSLSVRDFDPVHGDVIKHYKIRSLDNGGYYISPRITFPCISDMIKHYQKQADGLCRRLEKACISPKPQKPWDKDAWEIPRESIKLVKRLGAGQFGEVWMGYYNNSTKVAVKTLKPGTMSVQAFLEEANLMKTLQHDKLVRLYAVVTREEPIYIITEYMAKGSLLDFLKSDEGGKVLLPKLIDFSAQIAEGMAYIERKNYIHRDLRAANVLVSESLMCKIADFGLARVIEDNEYTAREGAKFPIKWTAPEAINFGCFTIKSDVWSFGILLYEIVTYGKIPYPGRTNADVMTALSQGYRMPRVENCPDELYDIMKMCWKEKAEERPTFDYLQSVLDDFYTATEGQYQQQPS

SEQ ID NO: 48 - protein, 541 aa, Artificial Sequence
  PDGFRa
GSKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFLS

SEQ ID NO: 49 - protein, 412 aa, Artificial Sequence
  PYK2
GSRLQGEHQGSLIIHPRKDGEKRNSLPQIPMLNLEARRSHLSESCSIESDIYAEIPDETLRRPGGPQYGIAREDVVLNRILGEGFFGEVYEGVYTNHKGEKINVAVKTCKKDCTLDNKEKFMSEAVIMKNLDHPHIVKLIGIIEEEPTWIIMELYPYGELGHYLERNKNSLKVLTLVLYSLQICKAMAYLESINCVHRDIAVRNILVASPECVKLGDFGLSRYIEDEDYYKASVTRLPIKWMSPESINFRRFTTASDVWMFAVCMWEILSFGKQPFFWLENKDVIGVLEKGDRLPKPDLCPPVLYTLMTRCWDYDPSDRPRFTELVCSLSDVYQMEKDIAMEQERNARYRTPKILEPTAFQEPPPKPSRPKYRPPPQTNLLAPKLQFQVPEGLCASSPTLTSPMEYPSPVNS

SEQ ID NO: 50 - protein, 637 aa, Artificial Sequence
  SYK
GSASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKIGTQGNVNFGGRPQLPGSHPATWSAGGIISRIKSYSFPKPGHRKSSPAQGNRQESTVSFNPYEPELAPWAADKGPQREALPMDTEVYESPYADPEEIRPKEVYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYYDVVNS

SEQ ID NO: 51 - protein, 354 aa, Artificial Sequence
  TIE2
GSRANVQRRMAQAFQNVREEPAVQFNSGTLALNRKVKNNPDPTIYPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAAS

SEQ ID NO: 52 - protein, 535 aa, Artificial Sequence
  YES
GSPAIKYRPENTPEPVSTSVSHYGAEPTTVSPCPSSSAKGTAVNFSSLSMTPFGGSSGVTPFGGASSSFSVVPSSYPAGLTGGVTIFVALYDYEARTTEDLSFKKGERFQIINNTEGDWWEARSIATGKNGYIPSNYVAPADSIQAEEWYFGKMGRKDAERLLLNPGNQRGIFLVRESETTKGAYSLSIRDWDEIRGDNVKHYKIRKLDNGGYYITTRAQFDTLQKLVKHYTEHADGLCHKLTTVCPTVKPQTQGLAKDAWEIPRESLRLEVKLGQGCFGEVWMGTWNGTTKVAIKTLKPGTMMPEAFLQEAQIMKKLRHDKLVPLYAVVSEEPIYIVTEFMSKGSLLDFLKEGDGKYLKLPQLVDMAAQIADGMAYIERMNYIHRDLRAANILVGENLVCKIADFGLARLIEDNEYTARQGAKFPIKWTAPEAALYGRFTIKSDVWSFGILQTELVTKGRVPYPGMVNREVLEQVERGYRMPCPQGCPESLHELMNLCWKKDPDERPTFEYIQSFLEDYFTATEPQYQPGENLS

SEQ ID NO: 53 - protein, 8 aa, Artificial Sequence
  FLAG Peptide
DYKDDDDK

SEQ ID NO: 54 - protein, 6 aa, Artificial Sequence
  LUMIO Tag
CCPGCC

SEQ ID NO: 55 - protein, 8 aa, Artificial Sequence
  Strep-Tag II
WSHPQFEK

SEQ ID NO: 56 - DNA, 1979 bp, Artificial Sequence
  Glutathione S-Transferase (GST)-IGF1R fusion protein
gaattctttttttttaaaccaccatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccagaaagagaaataacagcaggctggggaatggagtgctgtatgcctctgtgaacccggagtacttcagcgctgctgatgtgtacgttcctgatgagtgggaggtggctcgggagaagatcaccatgagccgggaacttgggcaggggtcgtttgggatggtctatgaaggagttgccaagggtgtggtgaaagatgaacctgaaaccagagtggccattaaaacagtgaacgaggccgcaagcatgcgtgagaggattgagtttctcaacgaagcttctgtgatgaaggagttcaattgtcaccatgtggtgcgattgctgggtgtggtgtcccaaggccagccaacactggtcatcatggaactgatgacacggggcgatctcaaaagttatctccggtctctgaggccagaaatggagaataatccagtcctagcacctccaagcctgagcaagatgattcagatggccggagagattgcagacggcatggcatacctcaacgccaataagttcgtccacagagaccttgctgcccggaattgcatggtagccgaagatttcacagtcaaaatcggagattttggtatgacgcgagatatctatgagacagactattaccggaaaggaggcaaagggctgctgcccgtgcgctggatgtctcctgagtccctcaaggatggagtcttcaccacttactcggacgtctggtccttcggggtcgtcctctgggagatcgccacactggccgagcagccctaccagggcttgtccaacgagcaagtccttcgcttcgtcatggagggcggccttctggacaagccagacaactgtcctgacatgctgtttgaactgatgcgcatgtgctggcagtataaccccaagatgaggccttccttcctggagatcatcagcagcatcaaagaggagatggagcctggcttccgggaggtctccttctactacagcgaggagaacaagctgcccgagccggaggagctggacctggagccagagaacatggagagcgtccccctggacccctcggcctcctcgtcctccctgccactgcccgacagacactcaggacacaaggccgagaacggccccggccctggggtgctggtcctccgcgccagcttcgacgagagacagccttacgcccacatgaacgggggccgcaagaacgagcgggccttgccgctgccccagtcttcgaccggctcgagttgttgtcctggctgttgcggccgcaccgtgactgactgacgagctagc

SEQ ID NO: 57 - protein, 648 aa, Artificial Sequence
  Glutathione S-Transferase (GST)-IGF1R protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTGSSCCPGCCGRTVTD

(19) United States
(12) Patent Application Publication
(10) Pub. No.: US 2007/0128633 A1
(43) Pub. Date: Jun. 7, 2007

(54) Cell-free protein expression systems and methods of use thereof

(75) Inventors: Sergey Alexeevich Zozulya, San Diego, CA (US); Douglas Eric McGrath, San Marcos, CA (US)

Correspondence Address: JONES DAY, 222 EAST 41ST ST, NEW YORK, NY, 10017, US

(73) Assignee: ChemBridge Research Laboratories, Inc., San Diego, CA (US)

(21) Appl. No.: 11/546,655

(22) Filed: Oct. 11, 2006

Related U.S. Application Data
(60) Provisional application No. 60/725,371, filed on Oct. 11, 2005.

(51) Int. Cl. C12Q 1/68; C12Q 1/48; C07H 21/04; C12P 21/06; C12N 9/12

(52) U.S. Cl. 435/6; 435/69.1; 435/15; 435/194; 435/320.1; 435/325; 536/23.2

(57) ABSTRACT

Provided herein are in vitro (cell-free) protein translation (IVT) systems for the expression of kinases. In particular, provided herein is an IVT system for the expression of a panel of protein tyrosine kinases (PTK), (e.g., receptor protein tyrosine kinases (RTK) and/or cytoplasmic tyrosine kinases (CTK)), and/or fragments thereof (e.g., kinase domains and/or active fragments thereof).

### CROSS-REFERENCE TO RELATED APPLICATIONS

This application claims priority to U.S. Ser. No. 60/725,371 filed Oct. 11, 2005.

### 1. FIELD

The subject matter provided herein relates to in vitro (cell-free) protein translation (IVT) systems for the expression of kinases. In particular, the present subject matter provided herein relates to IVT system for the expression of a panel of protein tyrosine kinases (PTK), (e.g., receptor protein tyrosine kinases (RTK) and/or cytoplasmic tyrosine kinases (CTK)), and/or fragments thereof (e.g., kinase domains and/or active fragments thereof).

### 2. BACKGROUND

2.1 Protein Tyrosine Kinases

According to the latest American Cancer Society's annual statistical report, released in January 2005, cancer has edged out heart disease as the leading cause of death in Americans under age 85. In 2002, the most recent year for which information is available, 476,009 Americans under 85 died of cancer compared with 450,637 who died of heart disease (those under 85 comprise 98.4 percent of the US population). Protein tyrosine kinases (PTK), which historically represented the majority of first discovered oncogenes, remain today one of the most important classes of oncology drug targets.

Protein kinases are enzymes which covalently modify proteins and peptides by the attachment of a phosphate group to one or more sites on the protein or peptide (for example, PTK phosphorylate tyrosine groups). The measurement of protein kinase activity is important since studies have shown that these enzymes are key regulators of many cell functions.

Over 500 protein kinases have been identified in the human genome (“kinome”) (Manning et al. (2002) Science. 298:1912). Based on the recent advances in deciphering the human genome, the family of human PTK consists of approximately 90 members (FIG. 1 and FIG. 2; Blume-Jensen and Hunter (2001) Nature, 411: 355-365; Robinson et al. (2000) Oncogene 19:5548-5557). This family can be divided in two major groups—receptor tyrosine kinases (RTK) and cytoplasmic (or non-receptor) tyrosine kinases (CTK)—and approximately 30 subfamilies based on structural similarity (see, e.g., Bolen et al. (1992) FASEB J. 6:3403-3409 (1992); Ullrich and Schlessinger (1990) Cell 61:203-212; Ihle (1995) Sem. Immunol. 7:247-254. PTKs are involved in regulation of many cellular processes, such as cell proliferation, survival and apoptosis. Enhanced activity of PTKs has been implicated in a variety of malignant and nonmalignant proliferative diseases. In addition, PTKs play a central role in the regulation of cells of the immune system. PTK inhibitors can thus impact a wide variety of oncologic and immunologic disorders. Such disorders may be ameliorated by selective inhibition of a certain receptor or non-receptor PTK, such as LCK, or due to the homology among PTK classes, by inhibition of more than one PTK by an inhibitor.

In some forms of cancer, a PTK mutation or structural alteration can increase the ability to proliferate, and thus, provides an advantage over surrounding cells. PTK of growth factor receptors, for instance, have been shown to be involved in the transformation of normal to cancerous cells (see, e.g., Rao (1996) Curr. Opin. Oncol. 8:516-524). PTK also play a role in the regulation of apoptosis or programmed cell death (see, e.g., Anderson (1997) Microbiol. Rev. 61:33). By activation of PTK, apoptosis mechanisms can be shut off and the elimination of cancerous cells is prevented. Thus, PTK exert their oncogenic effects via a number of mechanisms such as driving proliferation and cell motility and invasion. These PTK include HER2, BCR-ABL, SRC, and IGF1R.

There are many ways that a PTK can become oncogenic. For example, mutations (such as gain-of-function mutations) or small deletions in RTK and/or CTK are known to be associated with several malignancies (e.g., KIT/SCFR, EGFR/ERBB1, CSF-1R, FGFR1, FGFR3, HGFR, RET). Additionally, overexpression of certain types of PTK resulting, for example, from gene amplification has been shown to be associated with several common cancers in humans (e.g., EGFR/ERBB1, ERBB2/HER2/NEU, ERBB3/HER3, ERBB4/HER4, CSF-1R, PDGFR, FLK2/FLT3, FLT4/VEGFR3, FGFR1, FGFR2/K-SAM, FGFR4, HGFR, RON, EPHA2, PEHB2, EPHB4, AXL, TIE/TIE1). For a review of oncogenic kinase signaling, and mutated kinase genes that may be used in the systems and methods provided herein, see Blume-Jensen and Hunter (2001) Nature 411:355; Tibes et al (2005) Annu. Rev. Pharmacol. Toxicol. 45:357; Gschwind (2004) Nature Reviews 4:361; Paul and Mukhopadhay (2004) Int. J. Med. Sci (2004) 1:101.

The majority of PTKs are believed to be important drug targets, especially for anti-cancer therapy. Indeed, a very large proportion of known PTKs have been shown to be hyperactivated in cancer cells due to overexpression or constitutively activating mutations and to directly drive tumor growth. In addition, a subset of RTKs, such as vascular endothelial growth factor receptors (VEGFR), fibroblast growth factor receptors (FGFR) and some ephrin receptor (EPH) family members, is involved in driving angiogenesis while others (e.g., Met and discoidin domain receptor (DDR)) promote cell motility and invasion (e.g., metastasis).

The formation of new blood vessels, either from differentiating endothelial cells during embryonic development (vasculogenesis) or from pre-existing vessels during adult life (angiogenesis), is an essential feature of organ development, reproduction, and wound healing in higher organisms. Folkman and Shing, J. Biol. Chem., 267: 10931-10934 (1992); Reynolds et al., FASEB J., 6: 886-892 (1992); Risau et al., Development, 102: 471-478 (1988). Angiogenesis is implicated in the pathogenesis of a variety of disorders, including, but not limited to, solid tumors, intraocular neovascular syndromes such as proliferative retinopathies or age-related macular degeneration (AMD), rheumatoid arthritis, and psoriasis (Folkman et al., J. Biol. Chem. 267:10931-10934 (1992); Klagsbrun et al., Annu. Rev. Physiol. 53:217-239 (1991); and Garner A, “Vascular Diseases”. In: Pathobiology of ocular disease. A dynamic approach. Garner A, Klintworth G K, Eds. 2nd Edition Marcel Dekker, NY, pp 1625-1710 (1994)). For example, vascularization allows tumor cells in solid tumors to acquire a growth advantage and proliferative freedom as compared to normal cells. Accordingly, a correlation has been observed between microvessel density in tumors and patient survival with various cancers and tumors (Weidner et al., N Engl J Med 324:1-6 (1991); Horak et al., Lancet 340:1120-1124 (1992); and Macchiarini et al., Lancet 340:145-146 (1992)).

A number of RTK have been identified that govern discrete stages of vascular development (Folkman et al., Cell, 87:1153-1155 (1996); Hanahan, D., Science, 277:48-50 (1997); Risau, W., Nature, 386:671-674 (1997); Yancopoulos et al., Cell, 93:661-664 (1998)). For example, VEGFR2 (FLK1), a receptor for vascular endothelial growth factor (VEGF), mediates endothelial and hematopoietic precursor cell differentiation (Shalaby et al., Nature, 376:62-66 (1995); Carmeliet et al., Nature, 380:435-439 (1996); Ferrara et al., Nature 380:439-442 (1996)). VEGF also governs later stages of angiogenesis through ligation of VEGFR1 (FLT1) (Fong et al., Nature, 376:66-70 (1995)). Mice that lack VEGFR1 have disorganized vascular endothelium with ectopic occurrence of endothelial cells from the earliest stages of vascular development, suggesting that VEGFR1 signaling is essential for the proper assembly of endothelial sheets (Fong et al., supra). Another tyrosine kinase receptor, TEK (TIE2) (Dumont et al., Genes Dev. 8:1897-1909 (1994); Sato et al., Nature, 376:70-74 (1995)) and its ligands ANG1 (Davis et al., Cell 87:1161-1169 (1996); Suri et al., Cell 87:1171-1180 (1996)) and ANG2 (Maisonpierre et al., Science 277:55-60 (1997)) are involved in assembly of non-endothelial vessel wall components. TIE (TIE1) is involved in maintaining endothelial integrity, and its inactivation results in perinatal lethality due to edema and hemorrhage (Sato, et al., Nature 376:70-74 (1995)). The TEK pathway seems to be involved in maturation steps and promotes interactions between the endothelium and surrounding vessel wall components (Suri et al., supra; and Vikkula et al., Cell 87:1181-1190 (1996)).

The EPH tyrosine kinase subfamily appears to be the largest subfamily of transmembrane RTK (Pasquale et al., Curr. Opin. Cell Biol. 9:608-615 (1997); and Orioli and Klein, Trends in Genetics 13:354-359 (1997)). Ephrins and their EPH receptors govern proper cell migration and positioning during neural development, presumably through modulating intercellular repulsion (Pasquale, supra; Orioli and Klein, supra). Bidirectional signaling has been observed for some Ephrin-B/EPHB ligand/receptor pairs (Holland et al., Nature 383:722-725 (1996); and Bruckner et al., Science 275:1640-1643 (1997)). For example, Ephrin-A1 and Ephrin-B1 have been proposed to have angiogenic properties (Pandey et al., Science 268:567-569 (1995); and Stein et al., Genes Dev. 12:667-678 (1998)). Ephrin-B2, a ligand for EPHB4 receptor, was recently reported to mark the arterial compartment during early angiogenesis, and mice that lack Ephrin-B2 showed severe anomalies in capillary bed formation (Wang et al., Cell 93: 741-753 (1998)).

Thus, blocking tyrosine kinase activity represents a rational, targeted approach to cancer therapy. Additionally, because tyrosine kinases have a number of other diverse biological functions, such as regulation of metabolism, cell differentiation, inflammation, immune responses, and tissue morphogenesis, kinases are attractive for drug development outside oncology.

2.2 Profiling of PTK Inhibitors

Selective PTK inhibitors have shown to be successful in the treatment of various malignancies. Clinical experience with the first generation of PTK-targeting anti-cancer molecules revealed the need not just for developing inhibitors for additional tyrosine kinases, but also for novel molecules with different kinase inhibition profiles. For example, a potential clinical advantage of an inhibitor with a “tailor-made” profile of inhibition of several PTKs is the ability of simultaneous targeting of kinases driving deregulated cell proliferation, neovascularization, and/or invasion. On the other hand, a compound with a protein kinase inhibition pattern which is insufficiently selective or promiscuous may have unacceptable systemic toxicity. Lastly, there is a need for developing improved versions of some of the existing drugs that can target multiple mutant forms of PTK oncogenes for a “personalized” medicine approach that addresses specific subsets of oncology patients.

Efficient specificity profiling of inhibitor candidates emerging from high throughput screening (HTS) in the early stages of drug discovery process is currently hampered by the very significant time and expense of producing large panels of wild-type and mutant kinases in-house or resorting to commercial sources. A simple and affordable technology platform for quick specificity profiling of those early drug candidate leads would greatly facilitate addressing all of the aspects of new PTK inhibitor development mentioned above.

Illustrative cases of the personalized medicine aspect of PTK inhibitor specificity and the need to stratify patients towards their predisposition towards a particular therapeutic compound exist. An example of an oncogenic PTK often hyperactivated in cancers (such as gastrointestinal stromal tumors (GIST), mast cell leukemias, and testicular seminomas) is KIT or stem cell factor (SCF) receptor. Gain-of-finction point mutations in KIT are common in these malignancies. A recent study (Kemmer et al. (2004) Am. J. Pathol., 164; 305-313) identified six different point mutations in the catalytic domain of the kinase which occurred in 26% of seminomas. KIT variants containing 2 out of these 6 mutations were not inhibited by imatinib, a PTK inhibitor designed to target Abelson (ABL) tyrosine kinase, but which also inhibits platelet derived growth factor receptor (PDGFR), c-KIT (IC₅₀=0.1 μM) and some but not all PDGFR and KIT active site mutants (Capdeville et al. (2002) Nature Reviews: Drug Discovery, 1; 493-502). Another case study (Ma et al. (2002) Blood, 99: 1741-1744) describes KIT active site mutation D816V causing resistance to imatinib treatment in sporadic adult human mastocytosis.

The first systematic mutational analysis of complete human tyrosine kinase gene family in a human cancer type (colorectal) has recently been published (Bardelli et al. (2003) Science, 300: 949). This study has identified 45 non-synonymous mutations in 14 kinase genes after analyzing 35 colorectal cancer cell lines followed by another 147 colorectal cancers. While the authors did not experimentally test the effect of these mutations on kinase function, according to their analysis, positions of mutations within each protein, mostly in critical parts of the catalytic domain or juxtamembrane portions involved in dimerization, suggest that many of them may be activating in nature.

In summary, many dozens of upregulating mutations have been already identified for some of the representative oncogenic PTKs expressed in tumors. This may significantly increase the number of tyrosine kinases and kinase variants that need to be screened against a given drug candidate in order to provide a comprehensive compound selectivity profile. The ability to address this pharmacogenomics issue early in the pipeline of novel kinase inhibitor development is critically dependent on the availability of a quick and easy-to-use kinase expression platform to support biochemical profiling assays, such as that provided by the IVT system and methods provided herein.

2.3 Prior Art Kinase Expression Systems

Current expression systems of choice for eukaryotic protein kinases for HTS assays, X-ray crystallography and other research purposes are based on the infection of cultured insect cells by recombinant baculoviruses. The vast majority of commercially available purified functionally active recombinant kinases or their catalytic domains are produced in this way. Simpler and faster bacterial expression systems have a limited potential for functional expression of eukaryotic kinases, while the mammalian expression systems are more technically demanding and less productive than their insect cell counterparts.

Previously, the production of large panels of recombinant kinases in-house using conventional techniques represented a significant challenge for a typical bioscreening lab. For example, expression of a single kinase in insect cells including gene cloning, generation of recombinant baculoviral construct and viral stock, preparative infection and protein purification takes at least one month of work of a skilled bench scientist and can not be easily done in parallel for multiple kinases. Recombinant kinase preparations available from several vendors typically do not include multiple naturally occurring kinase mutants or even structurally and functionally comparable forms of one or more native kinases, in addition to being prohibitively expensive. The same is true for commercial compound specificity profiling services currently provided for panels containing only a limited number of kinases.

Thus, the subject matter provided herein satisfies the need for high-throughput expression techniques based on the use of the in vitro (i.e., cell-free) translation (IVT) systems which makes screening large panels of kinases feasible, efficient and affordable, such as for a small academic or industrial lab.

The most popular cell-free translation systems in the art consist of lysates or extracts from rabbit reticulocytes, wheat germ, and E. coli cells (S30 system). All are prepared from the corresponding source cells as crude extracts that contain the macromolecular components, amino acids, energy sources, energy regenerating systems, and various co-factors required for translation of added RNA in a test-tube.

The in vitro synthesis of proteins in cell extracts is a powerful research tool and has been widely used for analytical characterization of gene products for decades (Spirin, A. S. ed. (2002) Cell-Free Translation Systems, Springer Verlag, Berlin-Heidelberg-New York; Swartz, J. A. ed. (2003) Cell-Free Protein Expression. Springer Verlag, Berlin-Heidelberg-New York). Unfortunately, typical yields of recombinant proteins in standard rabbit reticulocyte lysate system run on the 1 ml scale, for example, are in the range of just a few micrograms, which complicates the use of this system for many research applications and assays.

There are many reports of successful functional expression of miscellaneous eukaryotic proteins in IVT systems, including the E. coli S30 cell-free system (Endo (2002) FEBS Letters, 514: 102-105; Endo and Sawasaki (2003) Biotechnology Advances, 21: 695-713; Spirin, A. S. ed. (2002) Cell-Free Translation Systems, Springer Verlag, Berlin-Heidelberg-New York; Swartz, J. A. ed. (2003) Cell-Free Protein Expression. Springer Verlag, Berlin-Heidelberg-New York). It is known that eukaryotic lysates, such as rabbit reticulocyte lysate and wheat germ extract, contain various protein folding co-factors and chaperonins, such as HSP90 involved in maturation of some kinases (Hartson et al. (1996) Biochemistry, 35: 13451-13459; Hutchison et al. (1992) J. Biol. Chem., 267(5): 2902-2908), and that the bacterial cell-free systems can be easily supplemented with those co-factors (Xu et al. (2002) Mol. Cell Biol., 22: 4419-4432; Yokoyama et al. (2003) Curr. Opin. Chem. Biol., 7:39-43).

There are also isolated reports of individual mammalian protein kinases expressed in an IVT system, for example, PKA (Foss et al. (1994) Eur. J. Biochem. 220(1):217-23), EF-2K kinase (Redpath et al. (1996) J. Biol. Chem. 271 (29): 17547-17554), Chk2 (Xu et al. (2002) Mol. Cell Boil, 22: 4419-4432), casein kinase I (Ambion, Inc.), v-mos (Herzog et al. (1990) J. Virol. 64(6): 3093-3096), Lck (Hartson et al. (1996) Biochemistry, 35: 13451-13459), Src (Hutchison et al. (1992) J. Biol. Chem., 267(5): 2902-2908; Sefton et al. (1979) J. Virol. 30:311-318.). Sawasaki et al. also expressed 439 cloned cDNAs encoding predicted kinases from the plant Arabidopsis thaliana in WGE system, and subsequent assays revealed 207 products having autophosphorylation activity (Sawasaki et al. (2004) Phytochemistry. 65:1549-1555).

Thus, there is a need for an IVT system, and methods of use thereof, for the IVT expression of panels of tyrosine kinases, and/or fragments thereof (e.g., kinase domains, and/or active fragments thereof) that may be used to screen test and identify compounds that modulate kinase activity. There is also a need for the above systems and methods where the kinases are isolated, relatively small and structurally uniform tyrosine kinase catalytic domains.

### 3. SUMMARY

As disclosed herein, IVT can be used to simply, rapidly and cost-effectively express PTK (e.g., RTK and/or CTK), and/or fragments thereof, such as large panels of PTK, and/or fragments thereof, wherein substantially all of the PTK, and/or fragments thereof, retain kinase activity. Thus, provided herein are in vitro (cell-free) protein translation (IVT) systems for the expression of kinases. In particular, provided herein is an IVT system for the expression of panels of PTK (e.g., RTK and/or CTK), and/or fragments thereof (e.g., kinase domains and/or active fragments thereof). In certain embodiments, substantially all of the PTK, and/or fragments thereof in the panel, are expressed in active form and/or have kinase activity. The subject matter provided herein also relates to methods of producing a panel of PTK (e.g., RTK and/or CTK) and/or fragments thereof (e.g., kinase domains and/or active fragments thereof), using the IVT system provided herein and IVT methods provided herein. In certain embodiments, a panel of PTK (e.g., RTK and/or CTK) and/or fragments thereof (e.g., kinase domains and/or active fragments thereof) are expressed in active form. Also provided herein are methods of screening a test compound, such as an agonist, antagonist (e.g., inhibitor) and/or other modulator of kinase activity of a PTK, using PTK and/or fragments thereof produced by the IVT systems and methods provided herein. In some embodiments, the methods of screening a drug are used for drug/patient profiling and/or “personalized medicine.” Also provided herein are methods of modulating kinase activity in a patient involving administering to the patient a test compound identified using the IVT systems and methods provided herein.

In one aspect, provided herein are methods for producing a panel of PTK (e.g., RTK and/or CTK), and/or fragments thereof (e.g., kinase domains, and/or active fragments thereof), wherein substantially all of said PTK in the panel have kinase activity, said method involving:
providing one or more polynucleotides that encode one or more polypeptides containing a tyrosine kinase domain, and/or fragment thereof,
optionally adding a first tag (e.g., a fluorescent tag) to the N-terminus of one or more of the polynucleotides,
optionally adding a second tag (e.g., an affinity purification tag) to the C-terminus of one or more of the polynucleotides,
translating the one or more polynucleotides in an in vitro cell-free translation (IVT) system, wherein the resulting one or more polypeptides contain a tyrosine kinase domain, and/or fragment thereof, having kinase activity.
   In certain embodiments, the one or more (such as about 5, 10, 15, 20, 30, 40, 50, 60, 70, 80, 90 or more, or such as about 100, 150, 200, 250, 300 350, 400, 450, 500 or more) polynucleotides encode for the same or different tyrosine kinases (e.g., PTK, RTK and/or CTK), and/or fragments thereof. In some embodiments, the one or more polynucleotides encode for tyrosine kinases, and/or fragments thereof, from different families and/or subfamilies of tyrosine kinases (see, e.g., FIG. 1, Manning et al. (2002) Science 298:1912). In another embodiment, the one or more polynucleotides encode for tyrosine kinases, and/or fragments thereof, from the same family and/or subfamily of tyrosine kinases. In an embodiment, the one or more polynucleotides encode for tyrosine kinases, and/or fragments thereof, wherein the tyrosine kinases, and/or fragments thereof, are in different forms (e.g., wild-type and/or mutant forms) of the same kinase, different forms of different kinases, or combinations thereof. In a specific embodiment, the one or more polynucleotides encode a tyrosine kinase domain (or an active fragment thereof). In one embodiment, a polynucleotide encoding the tyrosine kinase is a linear polynucleotide. In some embodiments, kinase arrays (e.g., a panel of kinases and/or fragments thereof) are produced from PCR DNA in an IVT system. In some embodiments, the in vitro translation system is wheat germ extract (WGE), rabbit reticulocyte lysate (RRL), E. coli S30 (S30) cell-free translation system, or a combination thereof.

In certain embodiments, the panel of tyrosine kinases contains about 5, 10, 15, 20, 30, 40, 50, 60, 70, 80, 90 or more, or such as about 100, 150, 200, 250, 300 350, 400, 450, 500 or more tyrosine kinases, including the same or different families or subfamilies of tyrosine kinases, the same or different forms (e.g., wild-type or mutant) of tyrosine kinases, and/or fragments thereof, that are human non-receptor tyrosine kinases, human receptor tyrosine kinases, or a combination thereof. In certain embodiments, the panel of tyrosine kinases contains tyrosine kinases, and/or fragments thereof, selected from EGFR, IGF1R, KIT, VEGFR1, FGFR1, TRKA, MET, EPHB4, AXL, TIE1, DDR1, RET, ROS, ALK, ROR1, MUSK, SRC, ABL, JAK1, ACK1, FAK, FES, BRK, TEC, ZAP70, BLK, BMX, BTK, CSFR, CSK, CTK, DDR2, EPHA2, EPHA4, FGFR2, FGFR4, FGR, FLT3, FRK, FYN, HER2, HER3, JAK2, JAK3, KDR, LCK, LYN, PDGFRα, PYK2, SYK, TIE2, YES and combinations thereof.

In a second aspect, provided herein are methods of screening for a modulator of tyrosine kinase activity (e.g., the activity of one or more tyrosine kinases, such as PTK, RTK and/or CTK), involving:
providing one or more polynucleotides that encode one or more polypeptides containing a tyrosine kinase domain, and/or fragment thereof,
optionally adding a first tag (e.g., a fluorescent tag) to the N-terminus of one or more of the polynucleotides,
optionally adding a second tag (e.g., an affinity purification tag) to the C-terminus of one or more of the polynucleotides,
translating the one or more polynucleotides in an in vitro cell-free translation (IVT) system, wherein the resulting one or more polypeptides contain a tyrosine kinase domain, and/or fragment thereof, having kinase activity,
contacting a test compound with the one or more polypeptides, and
detecting modulation of kinase activity the one or more polypeptides relative to kinase activity in the absence of test compound.
   In specific embodiments, the one or more polynucleotides encode a panel of PTK (e.g., RTK and/or CTK), and/or fragments thereof (e.g., kinase domains and/or active fragments thereof), wherein substantially all of said PTKs in the panel have kinase activity. In certain embodiments, the one or more (such as about 5, 10, 15, 20, 30, 40, 50, 60, 70, 80, 90 or more, or such as about 100, 150, 200, 250, 300 350, 400, 450, 500 or more) polynucleotides encode for the same or different tyrosine kinases (e.g., PTK, RTK and/or CTK), and/or fragments thereof. In some embodiments, the one or more polynucleotides encode for tyrosine kinases, and/or fragments thereof, from different families and/or subfamilies of tyrosine kinases (see, e.g., FIG. 1, Manning et al. (2002) Science 298:1912). In another embodiment, the one or more polynucleotides encode for tyrosine kinases, and/or fragments thereof, from the same family and/or subfamily of tyrosine kinases. In an embodiment, the one or more polynucleotides encode for tyrosine kinases, and/or fragments thereof, wherein the tyrosine kinases, and/or fragments thereof, are in different forms (e.g., wild-type and/or mutant forms) of the same kinase, different forms of different kinases, or combinations thereof. In a specific embodiment, the one or more polynucleotides encode a tyrosine kinase domain (or an active fragment thereof).

In certain embodiments, the panel of tyrosine kinases contains about 5, 10, 15, 20, 30, 40, 50, 60, 70, 80, 90 or more, or such as about 100, 150, 200, 250, 300 350, 400, 450, 500 or more tyrosine kinases, including the same or different families or subfamilies of tyrosine kinases, the same or different forms (e.g., wild-type or mutant) of tyrosine kinases, and/or fragments thereof, that are human non-receptor tyrosine kinases, human receptor tyrosine kinases, or a combination thereof. In certain embodiments, the panel of tyrosine kinases contains tyrosine kinases, and/or fragments thereof, selected from EGFR, IGF1R, KIT, VEGFR1, FGFR1, TRKA, MET, EPHB4, AXL, TIE1, DDR1, RET, ROS, ALK, ROR1, MUSK, SRC, ABL, JAK1, ACK1, FAK, FES, BRK, TEC, ZAP70, BLK, BMX, BTK, CSFR, CSK, CTK, DDR2, EPHA2, EPHA4, FGFR2, FGFR4, FGR, FLT3, FRK, FYN, HER2, HER3, JAK2, JAK3, KDR, LCK, LYN, PDGFRα, PYK2, SYK, TIE2, YES and combinations thereof.

In one embodiment, a polynucleotide encoding the tyrosine kinase is a linear polynucleotide. In some embodiments, kinase arrays (e.g., a panel of kinases and/or fragments thereof) are produced from PCR DNA in an IVT system. In certain embodiments, the one or more polynucleotides encoding the tyrosine kinases contain regulatory elements. In some embodiments, the in vitro translation system is a WGE, RRL, or S30 cell-free translation system, or a combination thereof.

Non-limiting examples of test compounds that can be used in the methods provided herein include any protein, polypeptide, peptide, organic molecule, inorganic molecule, antibody, pharmaceutical, and/or candidate pharmaceutical that are natural products or prepared synthetically, and/or any compound found in the U.S. Pharmacopoeia (USP) and/or Physician's Desk Reference (59^{th} ed., 2005; 60^{th} ed., 2006), which are incorporated herein by reference in their entirety.

In certain embodiments, a test compound is screened against a panel (e.g., more than 5, more than 10, more than 25, more than 50, more than 100, more than 150, more than 200, more than 250, more than 300, more than 350, more than 400, more than 450, or more than 500) of different kinases and/or different forms of kinases, and/or active fragments thereof, simultaneously or in sequence. In other embodiments, more than one test compound (e.g., more than 5, more than 10, more than 25, more than 50, or more than 100) is screened against a panel (e.g., more than 5, more than 10, more than 25, more than 50, more than 100, more than 150, more than 200, more than 250, more than 300, more than 350, more than 400, more than 450, or more than 500) of different kinases and/or different forms of kinases, and/or active fragments thereof, simultaneously or in sequence. In certain embodiments, the screens are completed in a single reaction on a single test plate or a single reaction on multiple test plates. In other embodiments, the screens are performed in multiple reactions on a single test plate or multiple reactions on multiple test plates. In certain embodiments, the screening methods provided herein are high-throughput screens (HTS), e.g., in a 384-well format.

In a third aspect, provided herein are methods for modulating (e.g., increasing, decreasing, inhibiting) tyrosine kinase activity (e.g., the activity of one or more tyrosine kinases, such as PTK, RTK and/or CTK) in a patient, involving:
providing one or more polynucleotides that encode one or more polypeptides containing a tyrosine kinase domain, and/or fragment thereof,
optionally adding a first tag (e.g., a fluorescent tag) to the N-terminus of the one or more polynucleotides,
optionally adding a second tag (e.g., an affinity purification tag) to the C-terminus of the one or more polynucleotides,
translating the one or more polynucleotides in an in vitro cell-free translation (IVT) system, wherein the resulting one or more polypeptides contain a tyrosine kinase domain, and/or fragment thereof, having kinase activity,
contacting a test compound with the one or more polypeptides,
detecting modulation of kinase activity of the one or more polypeptides relative to kinase activity in the absence of test compound, and
administering the test compound to the patient, wherein the test compound modulates kinase activity in the patient relative to kinase activity in the absence of test compound.

In specific embodiments, the one or more polynucleotides encode a panel of PTK (e.g., RTK and/or CTK), and/or fragments thereof (e.g., kinase domains and/or active fragments thereof), wherein substantially all of said PTKs in the panel have kinase activity. In some embodiments, the one or more (such as about 5, 10, 15, 20, 30, 40, 50, 60, 70, 80, 90 or more, or such as about 100, 150, 200, 250, 300, 350, 400, 450, 500 or more) polynucleotides encode for tyrosine kinases, and/or fragments thereof, from different families and/or subfamilies of tyrosine kinases (see, e.g., FIG. 1, Manning et al. (2002) Science 298:1912). In another embodiment, the one or more polynucleotides encode for tyrosine kinases, and/or fragments thereof, from the same family and/or subfamily of tyrosine kinases. In an embodiment, the one or more polynucleotides encode for tyrosine kinases, and/or fragments thereof, wherein the tyrosine kinases, and/or fragments thereof, are in different forms (e.g., wild-type and/or mutant forms) of the same kinase, different forms of different kinases, or combinations thereof. In a specific embodiment, the one or more polynucleotides encode a tyrosine kinase domain (or an active fragment thereof).

In certain embodiments, the panel of tyrosine kinases contains about 5, 10, 15, 20, 30, 40, 50, 60, 70, 80, 90 or more, or such as about 100, 150, 200, 250, 300 350, 400, 450, 500 or more tyrosine kinases, including the same or different families or subfamilies of tyrosine kinases, the same or different forms (e.g., wild-type or mutant) of tyrosine kinases, and/or fragments thereof, that are human non-receptor tyrosine kinases, human receptor tyrosine kinases, or a combination thereof. In certain embodiments, the panel of tyrosine kinases contains tyrosine kinases, and/or fragments thereof, selected from EGFR, IGF1R, KIT, VEGFR1, FGFR1, TRKA, MET, EPHB4, AXL, TIE1, DDR1, RET, ROS, ALK, ROR1, MUSK, SRC, ABL, JAK1, ACK1, FAK, FES, BRK, TEC, ZAP70, BLK, BMX, BTK, CSFR, CSK, CTK, DDR2, EPHA2, EPHA4, FGFR2, FGFR4, FGR, FLT3, FRK, FYN, HER2, HER3, JAK2, JAK3, KDR, LCK, LYN, PDGFRα, PYK2, SYK, TIE2, YES and combinations thereof.

In one embodiment, a polynucleotide encoding the tyrosine kinase is a linear polynucleotide. In some embodiments, kinase arrays (e.g., a panel of kinases and/or fragments thereof) are produced from PCR DNA in an IVT system. In certain embodiments, the one or more polynucleotides encoding the tyrosine kinases contain regulatory elements. In some embodiments, the in vitro translation system is a WGE, RRL, S30 cell-free translation system, or a combination thereof.

Non-limiting examples of test compounds that can be used in the methods provided herein include any protein, polypeptide, peptide, organic molecule, inorganic molecule, antibody, pharmaceutical, and/or candidate pharmaceutical that are natural products or prepared synthetically, and/or any compound found in the U.S. Pharmacopoeia (USP) and/or Physician's Desk Reference (59^{th} ed., 2005; 60^{th} ed., 2006), which are incorporated herein by reference in their entirety.

In certain embodiments, a test compound is screened against a panel (e.g., more than 5, more than 10, more than 25, more than 50, more than 100, more than 150, more than 200, more than 250, more than 300, more than 350, more than 400, more than 450, or more than 500) of different kinases and/or different forms of kinases, and/or active fragments thereof, simultaneously or in sequence. In other embodiments, more than one test compound (e.g., more than 5, more than 10, more than 25, more than 50, or more than 100) is screened against a panel (e.g., more than 5, more than 10, more than 25, more than 50, more than 100, more than 150, more than 200, more than 250, more than 300, more than 350, more than 400, more than 450, or more than 500) of different kinases and/or different forms of kinases, and/or active fragments thereof, simultaneously or in sequence. In certain embodiments, the screens are completed in a single reaction on a single test plate or a single reaction on multiple test plates. In other embodiments, the screens are performed in multiple reactions on a single test plate or multiple reactions on multiple test plates. In certain embodiments, the screening methods provided herein are high-throughput screens (HTS), e.g., in a 384-well format.

In a fourth aspect, provided herein are kits for screening for a modulator (an agonist, antagonist and/or any other type of activator or inhibitor) of tyrosine kinase activity (e.g., the activity of one or more tyrosine kinases in a panel of tyrosine kinases) containing:
one or more polynucleotides that encode for one or more polypeptides containing a tyrosine kinase domain, and/or fragment thereof, wherein said polynucleotides optionally further contains a first tag (e.g., a fluorescent tag) on the N-terminus of the one or more polynucleotide and/or a second tag (e.g., an affinity purification tag) on the C-terminus of the one or more polynucleotides, and
an in vitro translation system.
   In specific embodiments, the one or more polynucleotides encode a panel of PTK (e.g., RTK and/or CTK), and/or fragments thereof (e.g., kinase domains and/or active fragments thereof), wherein substantially all of said PTKs in the panel have kinase activity. In certain embodiments, the one or more (such as about 5, 10, 15, 20, 30, 40, 50, 60, 70, 80, 90 or more, or such as about 100, 150, 200, 250, 300 350, 400, 450, 500 or more) polynucleotides encode for the same or different tyrosine kinases (e.g., PTK, RTK and/or CTK), and/or fragments thereof. In some embodiments, the one or more polynucleotides encode for tyrosine kinases, and/or fragments thereof, from different families and/or subfamilies of tyrosine kinases (see, e.g., FIG. 1, Manning et al. (2002) Science 298:1912)). In another embodiment, the one or more polynucleotides encode for tyrosine kinases, and/or fragments thereof, from the same family and/or subfamily of tyrosine kinases. In an embodiment, the one or more polynucleotides encode for tyrosine kinases, and/or fragments thereof, wherein the tyrosine kinases, and/or fragments thereof, are in different forms (e.g., wild-type and/or mutant forms) of the same kinase, different forms of different kinases, or combinations thereof. In a specific embodiment, the one or more polynucleotides encode a tyrosine kinase domain (or an active fragment thereof).

In certain embodiments, the panel of tyrosine kinases contains about 5, 10, 15, 20, 30, 40, 50, 60, 70, 80, 90 or more, or such as about 100, 150, 200, 250, 300 350, 400, 450, 500 or more tyrosine kinases, including the same or different families or subfamilies of tyrosine kinases, the same or different forms (e.g., wild-type or mutant) of tyrosine kinases, and/or fragments thereof, that are human non-receptor tyrosine kinases, human receptor tyrosine kinases, or a combination thereof. In certain embodiments, the panel of tyrosine kinases contains tyrosine kinases, and/or fragments thereof, selected from EGFR, IGF1R, KIT, VEGFR1, FGFR1, TRKA, MET, EPHB4, AXL, TIE1, DDR1, RET, ROS, ALK, ROR1, MUSK, SRC, ABL, JAK1, ACK1, FAK, FES, BRK, TEC, ZAP70, BLK, BMX, BTK, CSFR, CSK, CTK, DDR2, EPHA2, EPHA4, FGFR2, FGFR4, FGR, FLT3, FRK, FYN, HER2, HER3, JAK2, JAK3, KDR, LCK, LYN, PDGFRα, PYK2, SYK, TIE2, YES and combinations thereof.

In one embodiment, a polynucleotide encoding the tyrosine kinase is a linear polynucleotide. In some embodiments, kinase arrays (e.g., a panel of kinases and/or fragments thereof) are produced from PCR DNA in an IVT system. In certain embodiments, the one or more polynucleotides encoding the tyrosine kinases contain regulatory elements. In some embodiments, the in vitro translation system is a WGE, RRL, S30 cell-free translation system, or a combination thereof.

3.1 Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The term “about” or “approximately” means within 20%, preferably within 10%, and more preferably within 5% (or 1% or less) of a given value or range.

As used herein, an “antagonist” or “inhibitor” of a kinase refers to a molecule that is capable of inhibiting or otherwise decreasing one or more of the biological activities of a target molecule, such as a kinase (e.g., a PTK, RTK and/or CTK). In some embodiments, an antagonist or inhibitor of a kinase may, for example, act by inhibiting or otherwise decreasing the activation of the kinase domain of the target molecule, thereby decreasing auto-, self- and/or transphosphorylation and/or the mediation of signal transduction relative to kinase activity in the absence of antagonist.

As used herein, an “agonist” or “activator” refers to a molecule which is capable of activating or otherwise increasing one or more of the biological activities of a target molecule, such as a kinase (e.g., a PTK, RTK and/or CTK). Agonists may, for example, act by activating or otherwise increasing the activity of the kinase domain of the target molecule, thereby increasing auto-, self- and/or transphosphorylation and/or the mediation of signal transduction relative to kinase activity in the absence of agonist.

As used herein, the terms “in vitro translation system,” or “cell-free translation system” and similar terms refer any polynucleotide translation or expression system, which excludes the presence of whole cells. Such systems can include extracts and/other proteins derived from whole cells, such as cell extracts. Such cell-free translations systems are known in the art, and non-limiting examples include WGE, RRL, and S10 expression systems.

As used herein, the term “polynucleotide” includes any DNA, RNA, or mRNA.

In the context of a polypeptide, the term “derivative” as used herein refers to a polypeptide that contains an amino acid sequence of a kinase domain or a fragment of a kinase domain polypeptide which has been altered by the introduction of amino acid residue substitutions, deletions or additions. The term “derivative” as used herein also refers to a kinase domain polypeptide, a fragment of a kinase domain polypeptide, or a kinase domain polypeptide which has been modified, i.e., by the covalent attachment of any type of molecule to the polypeptide. For example, but not by way of limitation, a kinase domain polypeptide or a fragment of a kinase domain polypeptide may be modified, e.g., by glycosylation, acetylation, pegylation, phosphorylation, amidation, derivatization by known protecting/blocking groups, proteolytic cleavage, linkage to a cellular ligand or other protein, etc. A derivative of a kinase domain polypeptide or a fragment of a kinase domain polypeptide may be modified by chemical modifications using techniques known to those of skill in the art, including, but not limited to specific chemical cleavage, acetylation, formylation, metabolic synthesis of tunicamycin, etc. Further, a derivative of a kinase domain polypeptide or a fragment of a kinase domain polypeptide may contain one or more non-classical amino acids. A polypeptide derivative possesses a similar or identical function as a kinase domain polypeptide or a fragment of a kinase domain described herein.

The term “effective amount” as used herein refers to the amount of a therapy (e.g., a test compound, such as a PTK agonist, antagonist, inhibitor or other modulator, identified by the methods provided herein) which is sufficient to increase, reduce, eliminate or otherwise modify the activity of a kinase and/or to enhance/improve the prophylactic or therapeutic effect(s) of another therapy (e.g., a therapy other than a test compound identified by the methods provided herein). Effective amounts of a given test compound will depend on a number of factors such as the disease being treated, the weight of the patient, etc., but will be readily determinable using routine methods well known to those in the art. For example, depending on the type and severity of the disease, from about 0.001 mg/kg to about 1000 mg/kg, such as about 0.01 mg to 100 mg/kg, or such as about 0.010 to 20 mg/kg of the test compound might be an initial candidate dosage for administration to the patient (such as a human patient), whether, for example, by one or more separate administrations, or by continuous infusion. For repeated administrations over several days or longer, depending on the condition, the treatment is repeated until a desired suppression of disease symptoms occurs or the desired improvement in the patient's condition is achieved. However, other dosage regimens may also be useful.

The term “fragment” as used herein refers to a peptide or polypeptide containing an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a polypeptide, such as a PTK (e.g., a RTK or CTK) or a kinase domain thereof (and/or active fragment). In a specific embodiment, a fragment of a kinase, such as a PTK (e.g., a RTK or CTK) retains kinase activity. In specific embodiments, the fragment of a kinase is a kinase domain, and/or fragment thereof, that retains kinase activity.

As used herein, one or more “modifications” to the amino acid residues of a kinase, kinase domain, and/or fragment thereof, refers to any mutation, substitution, insertion or deletion of one or more amino acid residues into the sequence of the kinase, kinase domain, and/or fragment thereof. In certain embodiments, kinase activity is retained following modification.

The terms “receptor” and “kinase receptor” refer to a protein having at least one phosphate accepting phenolic group. The protein is usually a receptor insofar as it has a ligand-binding extracellular domain, transmembrane domain and intracellular domain. The terms, “tyrosine kinase,” “tyrosine kinase receptor,” “receptor protein tyrosine kinase,” “protein tyrosine kinase,” and “PTK,” refer to types of kinases, wherein the intracellular domain contains a catalytic kinase domain, or active fragment thereof, and has one or more phosphate accepting tyrosine residues. See, for example, Ullrich and Schlessinger, Cell 81:203-212 (1990); Fantl et al., Annu. Rev. Biochem. 62:453-481 (1993); Mark et al., Journal of Biological Chemistry 269(14):10720-10728(1994); and WO 93/15201.

As used herein, “kinase activity” and similar terms refers to activity of the kinase domain of a PTK. For example, kinase activity of a PTK refers to the ability of the tyrosine kinase to auto- or self-phosphorylate and/or transphosphorylate a tyrosine residue on the same or another receptor, or another natural or synthetic substrate. In certain embodiments, an “active kinase domain” has the ability to phosphorylate and/or has already phosphorylated. Kinase activity may also be assessed by increases in cell signal transduction. By “autophosphorylation” is meant activation of the catalytic kinase domain of the PTK, whereby at least one intrinsic tyrosine residue is phosphorylated. Generally, autophosphorylation will result when an agonist molecule binds to the extracellular domain of the kinase receptor. Without being limited to any particular mechanism of action, it is thought that binding of the agonist molecule may result in oligomerization of the kinase receptor which causes activation of the catalytic kinase domain. In certain embodiments, a kinase, kinase domain, and/or fragment thereof, is activated (i.e., has kinase activity). As used herein, producing a kinase, kinase domain and/or fragment thereof in “active form” means that the kinase, kinase domain and/or fragment thereof has kinase activity.

As used herein, the terms “subject” and “patient” are used interchangeably. As used herein, a subject is can be a mammal such as a non-primate (e.g., cows, pigs, horses, cats, dogs, rats, etc.) or a primate (e.g., monkey and human) In specific embodiments, the patient is a human.

As used herein, “test compound” refers to any compound that is to be tested for its ability to modulate kinase activity of a kinase, kinase domain, and/or fragment thereof. Non-limiting examples of test compounds that can be used in the methods provided herein include any protein, polypeptide, peptide, nucleic acid, organic, inorganic, antibody, pharmaceutical, and/or candidate pharmaceutical that are natural products or prepared synthetically, and/or any compound found in the U.S. Pharmacopoeia (USP) and/or Physician's Desk Reference (59^{th} ed., 2005; 60^{th} ed., 2006), both of which are incorporated herein by reference in their entirety. Pharmaceutical compositions may be prepared and formulated in dosage forms by methods known in the art; for example, see Remington's Pharmaceutical Sciences (1990) Mack Publishing Co., Easton, Pa., which is hereby incorporated by reference in its entirety. A test compound may be either an agonist, antagonist, or other modulator of kinase activity. In specific embodiments, the test compound is an inhibitor of kinase activity.

The phrases “decreasing angiogenesis,” “inhibiting angiogenesis” and other similar phrases refer to the act of preventing or reducing blood vessel development in a patient. Angiogenesis may inhibited in a patient, for example, following administration of antagonist (or inhibitor) of kinase activity that is identified by the IVT screening methods provided herein.

The phrases “increasing angiogenesis,” “stimulating angiogenesis,” “promoting angiogenesis,” and other similar phrases refer to the increase of blood vessel development in a patient. Angiogenesis may stimulated in a patient, for example, following administration of agonist of kinase activity that is identified by the IVT screening methods provided herein.

The phrase, “disorder (or disease) characterized by excessive vascularization” and related phrases, includes, but is not limited to tumors, and especially solid malignant tumors, rheumatoid arthritis, psoriasis, atherosclerosis, diabetic and other retinopathies, retrolental fibroplasia, age-related macular degeneration, neovascular glaucoma, hemangiomas, thyroid hyperplasias (including Grave's disease), corneal and other tissue transplantation, and chronic inflammation.

Examples of a “disorder (or disease) characterized by excessive vascular permeability” include edema associated with brain tumors, ascites associated with malignancies, Meigs' syndrome, lung inflammation, nephrotic syndrome, pericardial effusion (such as that associated with pericarditis), and pleural effusion.

The expression “trauma to the vascular network” refers to trauma, such as injuries, to the blood vessels or heart, including the vascular network of organs, to which a mammal is subjected. Examples of such trauma include wounds, incisions, and ulcers, e.g., diabetic ulcers and wounds or lacerations of the blood vessels or heart. Trauma includes conditions caused by internal events as well as those that are imposed by an extrinsic agent such as a pathogen, which can be improved by promotion of angiogenesis. It also refers to the treatment of wounds in which vascularization or re-endothelialization is required for healing.

As used herein, the term “in combination” refers to the use of more than one therapy. The use of the term “in combination” does not restrict the order in which therapies are administered to a subject with a disease or disorder. A first therapy can be administered before (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject in need thereof. Any additional therapy can be administered in any order with the other additional therapies. In certain embodiments, the therapies (e.g., test compounds) identified using the IVT systems and methods provided herein can be administered in combination with one or more other therapies. In certain embodiments, the one or more other therapies is also an agonist, antagonist (e.g., inhibitor) or other modulator of kinase activity identified by the IVT systems and methods provided herein. In other embodiments, the one or more other therapies is not an agonist, antagonist (e.g., inhibitor) or other modulator of kinase activity identified by the IVT systems and methods provided herein. Non-limiting examples of therapies that can be administered in combination include analgesic agents, anesthetic agents, antibiotics, and/or immunomodulatory agents.

As used herein, the term “tag” refers to any type of moiety that is attached to, e.g., a polypeptide and/or a polynucleotide that encodes a kinase (e.g., a PTK, such as a RTK or a CTK). For example, a polynucleotide that encodes a PTK can contain one or more additional tag-encoding nucleotide sequences that encode a, e.g., a detectable moiety or a moiety that aids in affinity purification. When translated, the tag and the PTK protein can be in the form of a fusion protein.

As used herein, the term “detectable” or “detection” with reference to a tag refers to any tag that is capable of being visualized or wherein the presence of the tag is otherwise able to be determined and/or measured (e.g., by quantitation). A non-limiting example of a detectable tag is a fluorescent tag, such as a GFP or LUMIO™ (Invitrogen Corp.) tag.

A used herein, the terms “panel,” “panel of PTK,” “panel of tyrosine kinases,” and other similar terms refer to more than about 5, 10, 20, 30, 50, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 tyrosine kinases and/or fragments thereof (e.g., a kinase domain, or a fragment of a kinase domain that has kinase activity). In one embodiment, the tyrosine kinases, and/or fragments thereof, in the panel are from different families and/or subfamilies of tyrosine kinases (see, e.g., FIG. 1, Manning et al. (2002) Science 298:1912)). In another embodiment, the tyrosine kinases, and/or fragments thereof, in the panel are from the same family and/or subfamily of tyrosine kinases. In an embodiment, the panel of tyrosine kinases, and/or fragments thereof, contain different forms (e.g., wild-type and/or mutant forms) of the same kinase, different forms of different kinases, or combinations thereof. In a specific embodiment, the panel of tyrosine kinases contains tyrosine kinase domains (or an active fragment thereof). In some embodiments, the panel contains multiple naturally occurring kinase mutants, synthetically prepared kinase mutants, structurally comparable forms of one or more native (e.g., wild-type) kinases, and/or functionally comparable forms of one or more native kinases. In certain embodiments, the panel of tyrosine kinases contains about 5, 10, 15, 20, 30, 40, 50, 60, 70, 80, 90 or more, or such as about 100, 150, 200, 250, 300 350, 400, 450, 500 or more tyrosine kinases, including the same or different families or subfamilies of tyrosine kinases, the same or different forms (e.g., wild-type or mutant) of tyrosine kinases, and/or fragments thereof, that are human non-receptor tyrosine kinases, human receptor tyrosine kinases, or a combination thereof. In certain embodiments, the panel of tyrosine kinases contains tyrosine kinases, and/or fragments thereof, selected from EGFR, IGF1R, KIT, VEGFR1, FGFR1, TRKA, MET, EphB4, AXL, TIE1, DDR1, RET, ROS, ALK, ROR1, Musk, SRC, ABL, JAK1, ACK1, FAK, FES, BRK, TEC, ZAP70, BLK, BMX, BTK, CSFR, CSK, CTK, DDR2, EPHA2, EPHA4, FGFR2, FGFR4, FGR, FLT3, FRK, FYN, HER2, HER3, JAK2, JAK3, KDR, LCK, LYN, PDGFRα, PYK2, SYK, TIE2, YES and combinations thereof.

As used herein “substantially all” refers to refers to at least about 60%, at least about 70%, at least about 75%, at least about 80%, at least about 85%, at least about 90%, at least about 95%, at least about 98%, at least about 99%, or about 100%.

As used herein, the term “therapeutically effective amount” means the amount of the subject composition that will elicit the biological or medical response of a tissue, system, animal or human that is being sought by the researcher, veterinarian, medical doctor or other clinician.

As used herein, the term “composition” is intended to encompass a product containing the specified ingredients in the specified amounts, as well as any product which results, directly or indirectly, from combination of the specified ingredients in the specified amounts.

The term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans. In specific embodiments, “pharmaceutically acceptable” means that the carrier, diluent or excipient must be compatible with the other ingredients of the formulation and not deleterious to the recipient thereof.

As used herein, “administer” or “administration” refers to the act of injecting or otherwise physically delivering a substance as it exists outside the body (e.g., a PTK agonist or antagonist identified using the methods of the invention) into a patient, such as by mucosal, intradermal, intravenous, intramuscular delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof. In specific embodiments, the terms “administration of” and/or “administering a” compound and similar terms should be understood to mean providing a compound provided herein to the patient in need of treatment.

### 4. BRIEF DESCRIPTION OF THE FIGURES

FIG. 1 depicts human receptor protein tyrosine kinases (RTKs). All subfamily members are listed under the prototypical structure drawings indicating arrangements of modular domains. Kinase domains are indicated by a light gray box outlined by a thicker black line (located beneath the cell membrane (horizontal line) in this figure). Cysteine-rich domains are indicated by a light gray box outlined by a thinner black line (located above the cell membrane in this figure). FNIII-like domains are indicated by a dark gray box (located above the cell membrane in this figure). Abbreviations of the prototypic receptors: EGFR, epidermal growth factor receptor; INSR, insulin receptor; PDGFR, platelet-derived growth factor receptor; VEGFR, vascular endothelial growth factor receptor; FGFR, fibroblast growth factor receptor; CCK, colon carcinoma kinase; EPH, ephrin receptor; AXL, a TYRO3 PTK; TIE, tyrosine kinase receptor in endothelial cells; RYK, receptor related to tyrosine kinases; DDR, discoidin domain receptor; RET, rearranged during transfection; ROS, RTK expressed in some epithelial cell types; LTK, leukocyte tyrosine kinase; ROR, receptor orphan; MUSK, muscle-specific-kinase; LMR, Lemur kinase.

FIG. 2 depicts human cytoplasmic protein-tyrosine kinases (CTKs).

FIG. 3 is a schematic representation of the linked in vitro transcription and translation procedure using rabbit reticulocyte lysate (Ambion, Inc.; available on the worldwide web at ambion.com).

FIG. 4 is a schematic representation of the addition of regulatory regions and tags to a protein expression cassette by overlap extension PCR (See Example 6.1 below).

FIGS. 5A-5Z shows the amino acid sequences for 26 tyrosine kinase domain fragments selected for expression in an expression cassette: (A) EGFR, (B) IGF1R, (C) Kit, (D) VEGFR1, (E) FGFR1, (F) TRKA, (G) MET, (H) EPHB4, (I) AXL, (J) TIE1, (K) DDR1, (L) RET, (M) ROS, (N) ALK, (0) ROR1, (P) MUSK, (Q) SRC, (R) ABL, (S) JAK1, (T) ACK1, (U) FAK, (V) FES, (W) BRK, (X) TEC, (Y) ZAP70, and (Z) BLK.

FIG. 6A-6Z shows the amino acid sequences for 26 tyrosine kinase domain fragments selected for expression in an expression cassette: (A) BMX, (B) BTK, (C) CSFR, (D) CSK, (E) CTK, (F) DDR2, (G) EPHA2, (G) EPHA4, (I) FGFR2, (J) FGFR4, (K) FGR, (L) FLT3, (M) FRK, (N) FYN, (0) HER2, (P) HER3, (Q) JAK2, (R) JAK3, (S) KDR, (T) LCK, (U) LYN, (V) PDGFRα, (W) PYK2, (X) SYK, (Y) TIE2 and (Z) YES.

FIG. 7 is a schematic representation one embodiment of the methods provided herein.

FIG. 8 depicts the nucleotide and amino acid sequence of an exemplary expression cassette encoding glutathione S-transferase (GST)-IGF1R fusion protein (see Example 6.1 below).

FIGS. 9A-9B depicts the expression and purification of the GST-IGF1R fusion protein described in Example 6.1. (A) The total translation sample of a RRL reaction following staining with a anti-phosphotyrosine monoclonal antibody (4G10). Lanes 1 and 2 show GST-IGF1R fusion proteins produced in an RRL IVT reaction. Lane 3 shows a negative control RRL translation reaction with no template. Lane 4 (M) shows the molecular weight marker. (B) A glutathione-sepharose purified sample of a RRL translation reaction following staining with anti-GST monoclonal antibody (left panel) or anti-phosphotyrosine monoclonal antibody (right panel). Lane A shows a negative control RRL translation reaction with no template. Lane B shows the GST-IGF1R fusion proteins produced in an RRL IVT reaction.

FIG. 10 depicts a Western blot analysis of GST-fusions of various tyrosine kinases produced in RRL in vitro translation system. The fusion proteins were precipitated from IVT mixtures with MagneGST™ glutathione particles and detected on a blot with anti-GST monoclonal antibody (mAb).

FIG. 11 shows ALPHASCREEN™ phosphotyrosine assay of the recombinant GST-IGF1R kinase produced in rabbit reticulocyte lysate in vitro translation system, as described in Example 6.1. Results of three independent translation experiments are shown.

FIG. 12 shows ALPHASCREEN™ phosphotyrosine assay of the recombinant GST-ABL kinase produced in rabbit reticulocyte lysate in vitro translation system, as described in Example 6.2.

FIGS. 13A-13B depicts exemplary kinase inhibition profiling using a panel of 23 tyrosine kinases for two examplary compounds, (A) “Compound A” and (B) “Compound B,” tested at 1 μM compound concentration.

### 5. DETAILED DESCRIPTION

In vitro (cell-free) protein translation (IVT) systems can be used to simply, rapidly and cost-effectively express panels of kinases (e.g., PTK), and/or fragments thereof (e.g., kinase domains, and/or active fragments thereof), which retain kinase activity. Thus, provided herein are IVT systems for the expression of kinases. In particular, provided herein is an IVT system for the expression of a panel of PTK (e.g., RTK and/or CTK) and/or fragments thereof (e.g., kinase domains and/or active fragments thereof). In certain embodiments, substantially all of the PTK, and/or fragments thereof, in the panel have kinase activity. In other embodiments, the panel contains active catalytic kinase domains, and/or active fragments thereof. Also provided herein are methods of producing panels of PTK (e.g., RTK and/or CTK) and/or fragments thereof (e.g., kinase domains and/or active fragments thereof), including their multiple mutant versions specific for one or more malignancies using the IVT system provided herein. In certain embodiments, substantially all of the PTK in the panel have kinase activity. In certain embodiments, the panels of PTK can be used for, e.g., small molecule kinase inhibitor high-throughput screening (HTS) using modem biochemical assays. Further provided herein are methods of screening a test compound for its ability to modulate kinase activity of a PTK, and/or fragments thereof, produced by the IVT system and methods provided herein. In some embodiments, the test compound in an agonist, antagonist (e.g., inhibitor) or other modulator of kinase activity. In certain embodiments, the methods provided herein are used for drug/patient profiling and/or “personalized medicine.” Also provided herein are methods of modulating kinase activity in a patient involving administering to the patient a test compound identified using the IVT system and methods provided herein.

5.1 Methods of Producing a Tyrosine Kinase

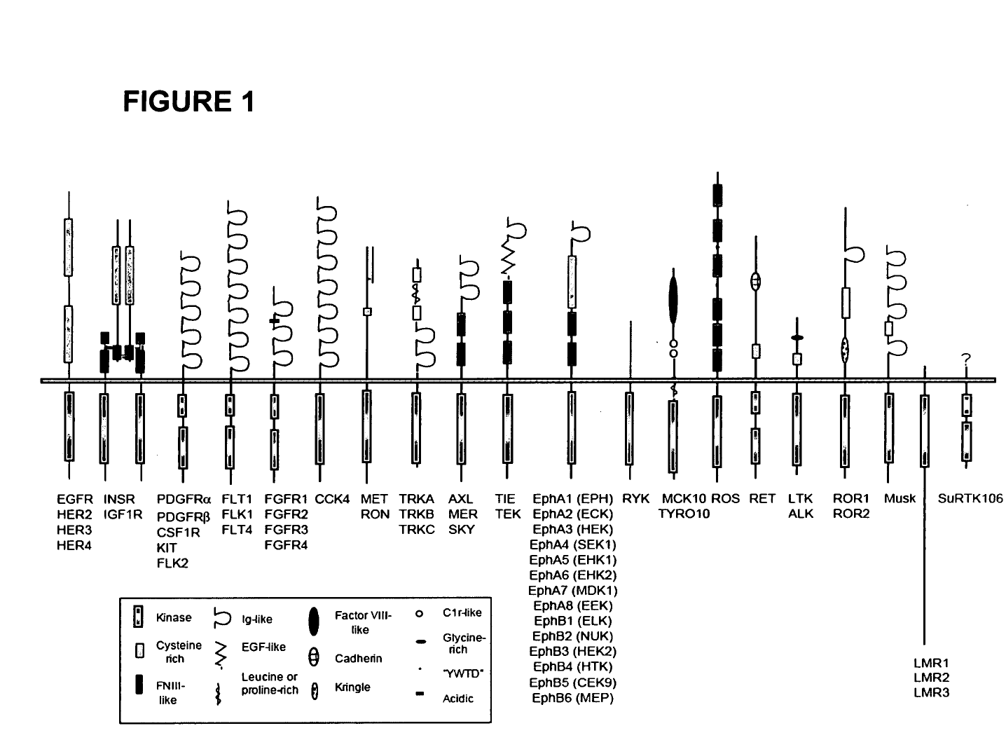

In one aspect, provided herein are methods for producing a panel of PTK (e.g., RTK and/or CTK), and/or fragments thereof (e.g., kinase domains, and/or active fragments thereof), wherein substantially all of said PTK in the panel have kinase activity, said method involving:
providing one or more polynucleotides that encode one or more polypeptides containing a tyrosine kinase domain, and/or fragment thereof,
optionally adding a first tag (e.g., a fluorescent tag) to the N-terminus of one or more of the polynucleotides,
optionally adding a second tag (e.g., an affinity purification tag) to the C-terminus of one or more of the polynucleotides,
translating the one or more polynucleotides in an in vitro cell-free translation (IVT) system, wherein the resulting one or more polypeptides contain a tyrosine kinase domain, and/or fragment thereof, having kinase activity.
   In certain embodiments, the one or more (such as about 5, 10, 15, 20, 30, 40, 50, 60, 70, 80, 90 or more, or such as about 100, 150, 200, 250, 300 350, 400, 450, 500 or more) polynucleotides encode for the same or different tyrosine kinases (e.g., PTK, RTK and/or CTK), and/or fragments thereof. In some embodiments, the one or more polynucleotides encode for tyrosine kinases, and/or fragments thereof, from different families and/or subfamilies of tyrosine kinases (see, e.g., FIG. 1, Manning et al. (2002) Science 298:1912)). In another embodiment, the one or more polynucleotides encode for tyrosine kinases, and/or fragments thereof, from the same family and/or subfamily of tyrosine kinases. In an embodiment, the one or more polynucleotides encode for tyrosine kinases, and/or fragments thereof, wherein the tyrosine kinases, and/or fragments thereof, are in different forms (e.g., wild-type and/or mutant forms) of the same kinase, different forms of different kinases, or combinations thereof. In a specific embodiment, the one or more polynucleotides encode a tyrosine kinase domain (or an active fragment thereof).

5.1.1 Selection of Kinase Domains

In certain embodiments, the panel of tyrosine kinases contains about 5, 10, 15, 20, 30, 40, 50, 60, 70, 80, 90 or more, or such as about 100, 150, 200, 250, 300 350, 400, 450, 500 or more tyrosine kinases, including the same or different families or subfamilies of tyrosine kinases, the same or different forms (e.g., wild-type or mutant) of tyrosine kinases, and/or fragments thereof. In some embodiments, the panel of tyrosine kinases contains tyrosine kinases, and/or fragments thereof, that are human non-receptor tyrosine kinases, human receptor tyrosine kinases, or a combination thereof.

In one embodiment, the panel of tyrosine kinases contains a human non-receptor tyrosine kinase, and/or fragment thereof, that is a member of the ABL family (e.g., ABL1 (ABL), ARG (ABL2, ABLL)), ACK family (e.g., ACK1 (ACK2(b), Cdgip(m)), TNK1), CSK family (e.g., CSK (CYL), MATK (CTK, HYL, CHK, LSK, Ntk(m))), FAK family (FAK (PTK2, Fadk(m)), PYK2 (PTK2B, CAKbeta, RAFTK, FAK2, PKB)), FES family (e.g., FER (TYK3, Fert1/2(m)), FES (FPS)), FRK family (e.g., BRK (PTK6, Sik(m)), FRK (RAK, Bsk(m), IYK(m)), SRMS (SRM)), JAK family (e.g., JAK1, JAK2, JAK3 (L-JAK), TYK2 (JYK1)), SRC-A family (e.g., FGR (SRC2), FYN (SLK, SYN), SRC, YES1), SRC-B family (e.g., BLK, HCK (JTK9, Bmk(m), HCTK), LCK (Tck(m)), LYN), TEC family (e.g., (BMX (ETK, PSCTK2), BTK (ATK, PSCTK1, AGMX1, IMD1), ITK (EMT, Tsk(m), PSCTK2), TEC (PSCTK4), TXK (PCTK5, BTKL, Rlk(m))), and/or SYK family (e.g., SYK, ZAP70 (SRK, STD)).

In another embodiment, the panel of tyrosine kinases contains a human receptor tyrosine kinase, and/or fragment thereof, that is a member of the ALK family (e.g., ALK (Kil), LTK (TYK1)), AXL family (e.g., AXL (UFO, Tyro7(r), Ark(m)), MER (MERTK, NYK, Eyk(ch), TYRO3 (RSE, SKY, BRT, DTK, TIF)), DDR family (e.g., DDR1 (CAK, TRKE, NEP, NTRK4, EDDR1, PTK3, MCK10), DDR2 (TKT, TYRO10, NTRKR3)), EGFR family (e.g., EGFR (ERBB, ERBB1), ERBB2 (HER2, Neu(r), NGL), ERBB3 (HER3), ERBB4 (HER4)), EPH family (e.g., EPHA1 (EPH, EPHT), EPHA2 (ECK, Sek2(m), Myk2(m)), EPHA3 (HEK, ETK1, Tyro4(r), Mek4(m), Cek4(ch)), EPHA4 (HEK8, Tyro1(r), Sek1(m), Cek8(ch)), EPHA5 (HEK7, Ehk1(r), Bsk(r), Cek7(ch)), EPHA6 (DKFZp434C1418), Ehk2(r)), EPHA7 (HEK11, Mdk1(m), Ebk(m), Ehk3(r), Cek11(ch)), EPHA8 (HEK3, KIAA1459, Eek(r), Cek10(ch)), EPHB1 (NET, EPHT2, HEK6, Elk(r), Cek6(ch)), EPHB2 (HEK5, ERK, DRT, EPHT3, Tyro5(r), Nuk(m), Sek3(m), Cek5(ch)), EPHB3 (HEK2, Tyro6, Mdk5(m), Sek4(m)), EPHB4 (HTK, Tyro11(r), Mdk2(m), Myk1(m)), EPHB5 (CEK9), EPHB6 (HEP, Mep(m), Cek1(ch)), EPHX)), FGFR family (e.g., FGFR1 (FLT2, bFGFR, FLG, N-SAM), FGFR2 (KGFR, K-SAM, Bek(m), CFD1, JWS, Cek3(ch), FGFR3 (HBGFR, ACH, Cek2(ch)), FGFR4), INSR family (e.g., IGF1R (JTK13), INSR (IR), INSRR (IRR)), MET family (e.g., MET (HGFR), RON (MST1R, CDw136, Fv2(m), STK(m), SEA(ch)), MUSK family (e.g., MUSK (Nsk2(m), Mlk1(m), Mlk2(m)), PDGFR family (e.g., CSF1R (FMS, C-FMS, CD115), FLT3 (FLK2, STK1, CD135), KIT (Sfr(m), CKIT), PDGFRA, PDGFRB (PDGFR, JTK12)), PTK7 family (e.g., PTK7 (CCK4, KLG(ch)), RET family (e.g., RET (MEN2A/B, HSCR1, MTC1)), ROR family (e.g., ROR1 (NTRKR1), ROR2 (NTRKR2)), ROS family (e.g., ROS1 (MCF3)), RYK family (e.g., RYK (Vik(m), Mrk(m))), TIE family (e.g., TEK (TIE2), TIE (TIE1, JTK14)), TRK family (e.g., NTRK1 (TRK, TRKA), NTRK2 (TRKB), NTRK3 (TRKC)), VEGFR family (e.g., VEGFR1 (FLT1), VEGFR2 (KDR, FLK1), VEGFR3 (FLT4, PCL)), AATYK family (e.g., AATYK (AATK, KIAA0641, LMR1), AATYK2 (KIAA1079, LMR2), AATYK3 (LMR3)), and/or SuRTK106 family (e.g., SuRTK106).

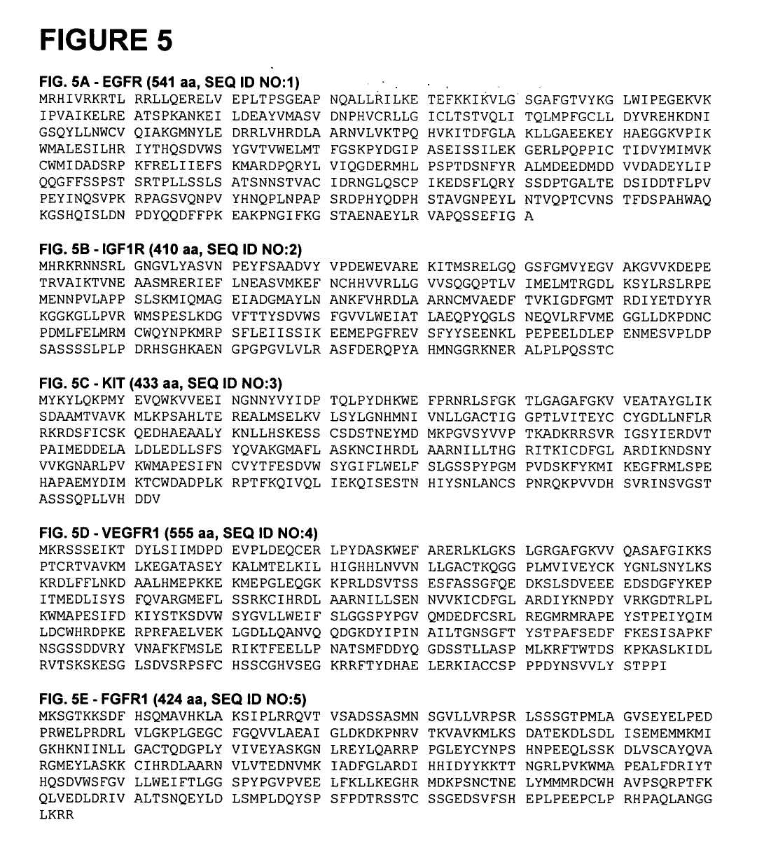

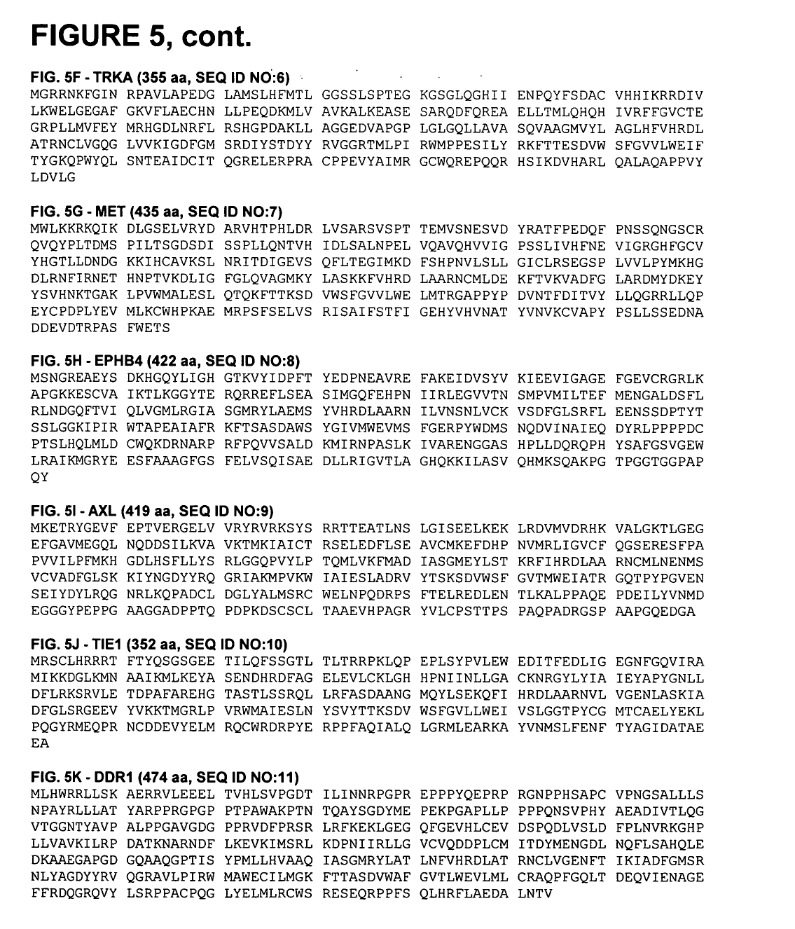

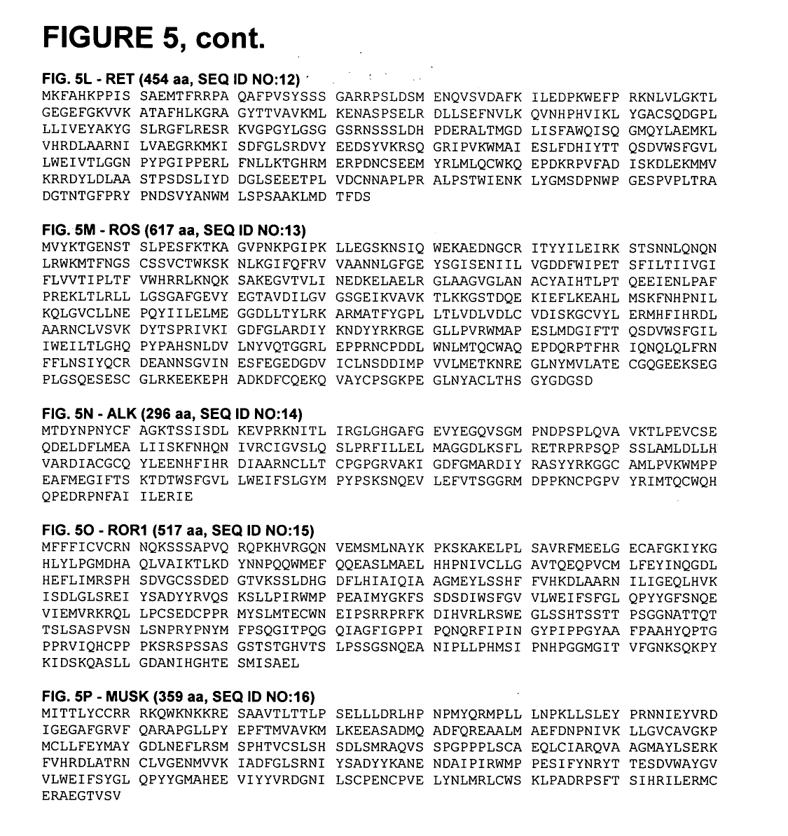

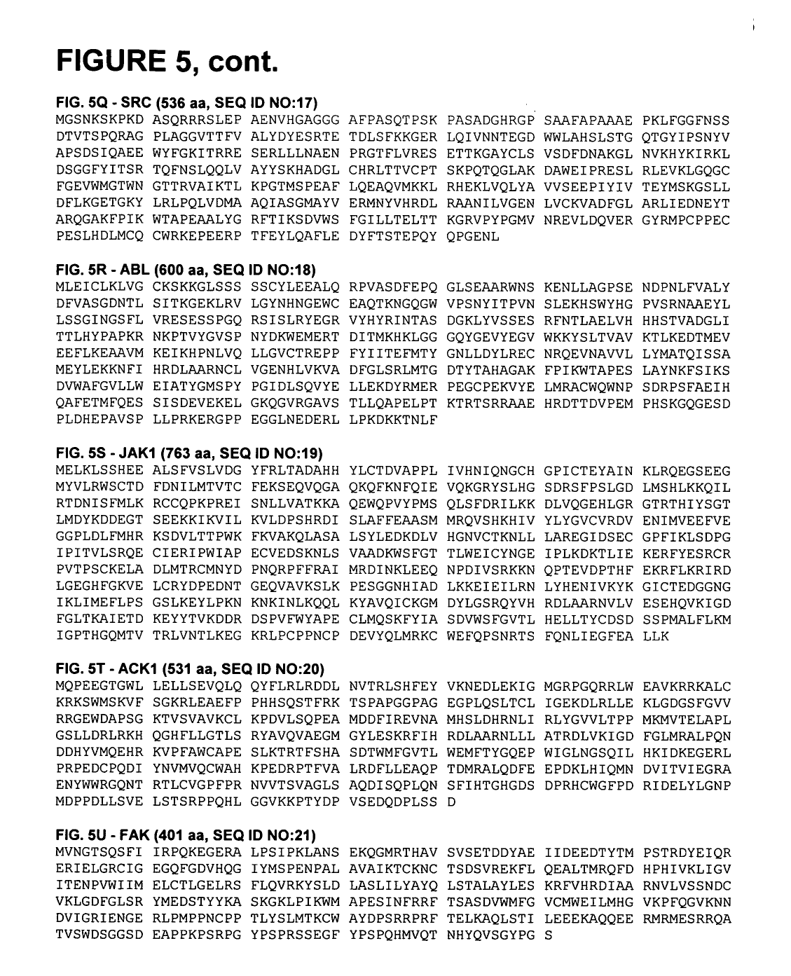

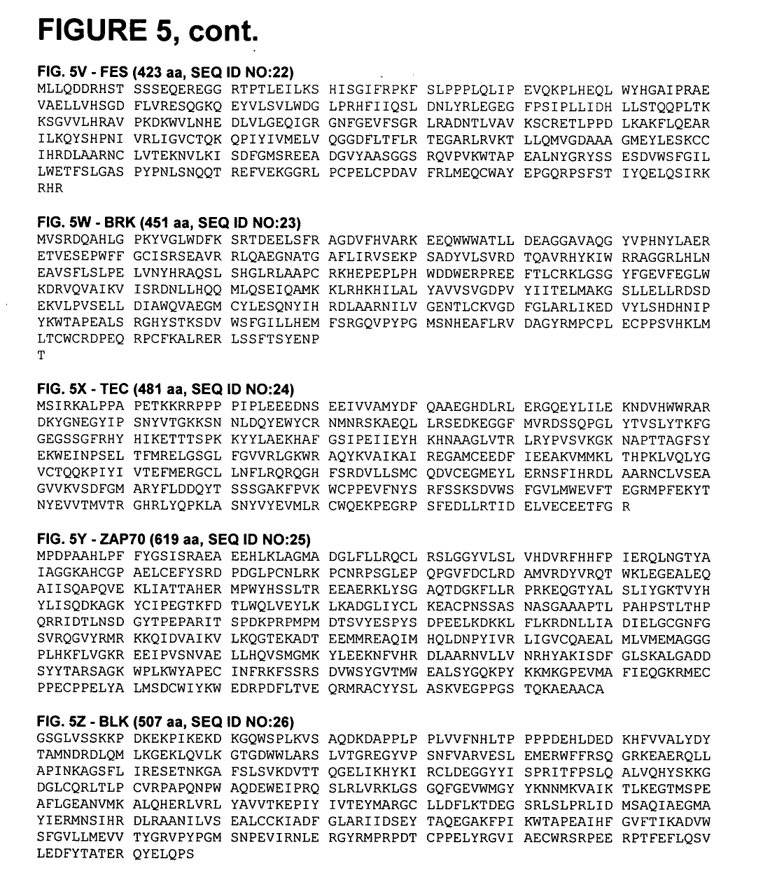

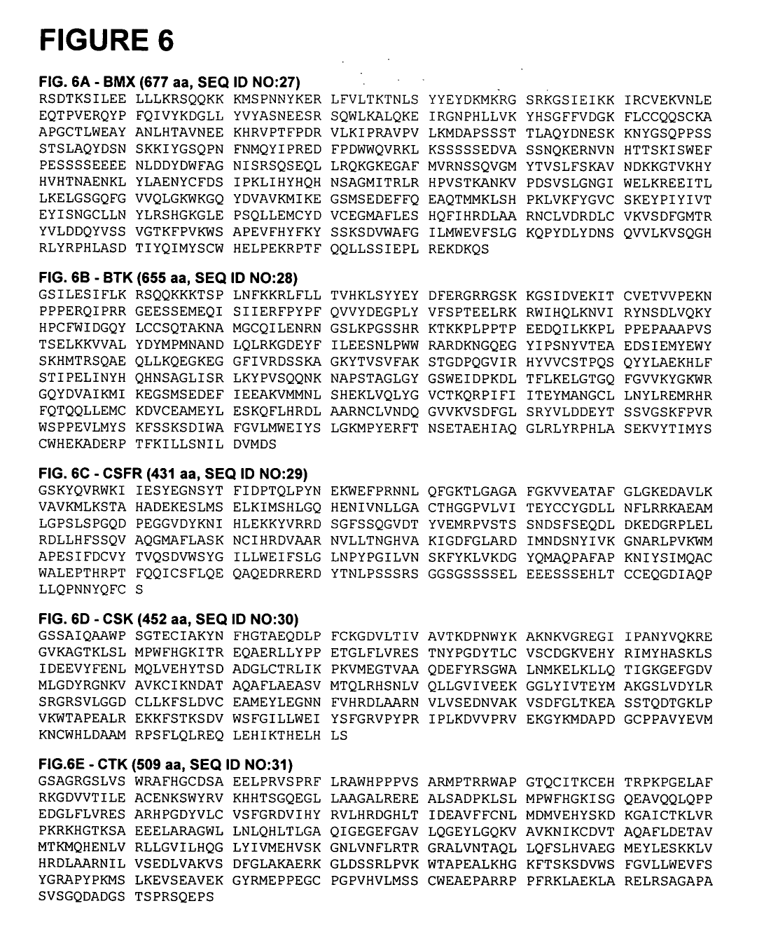

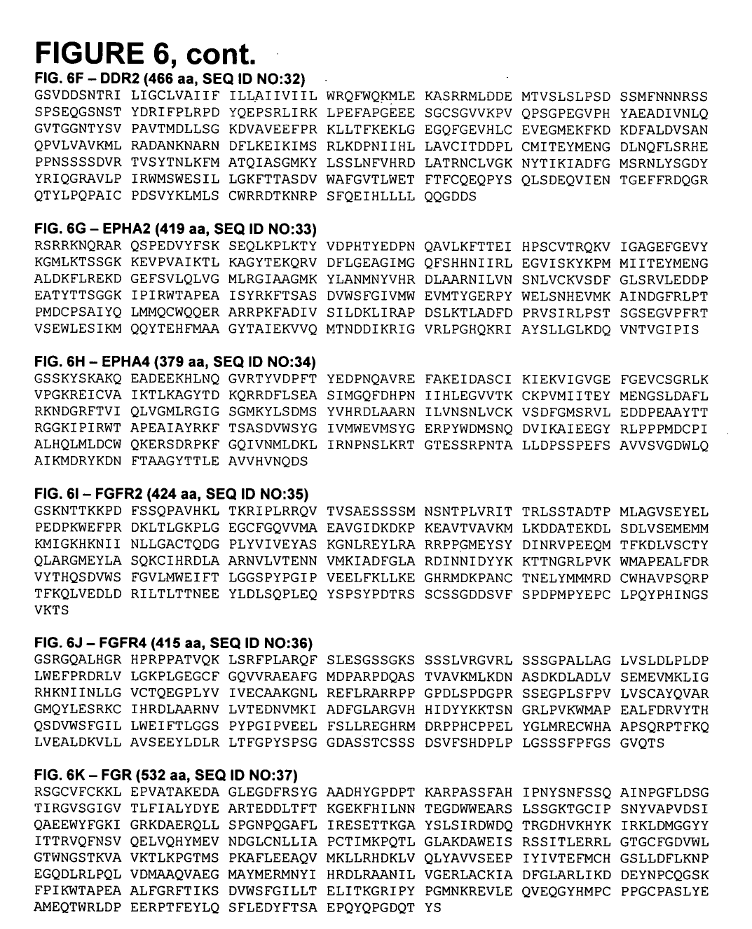

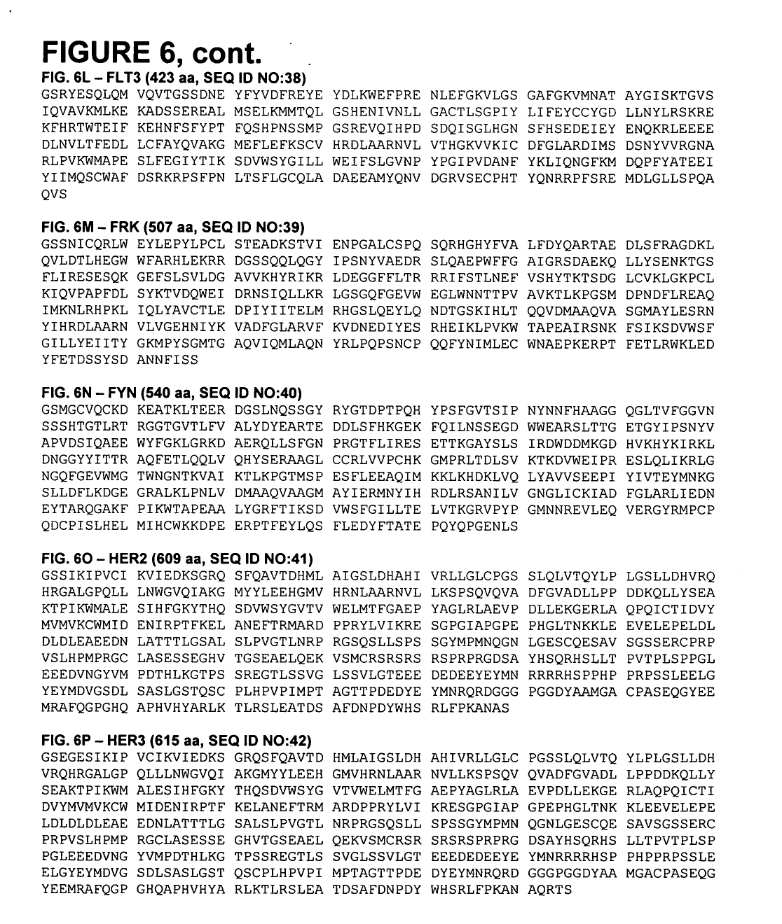

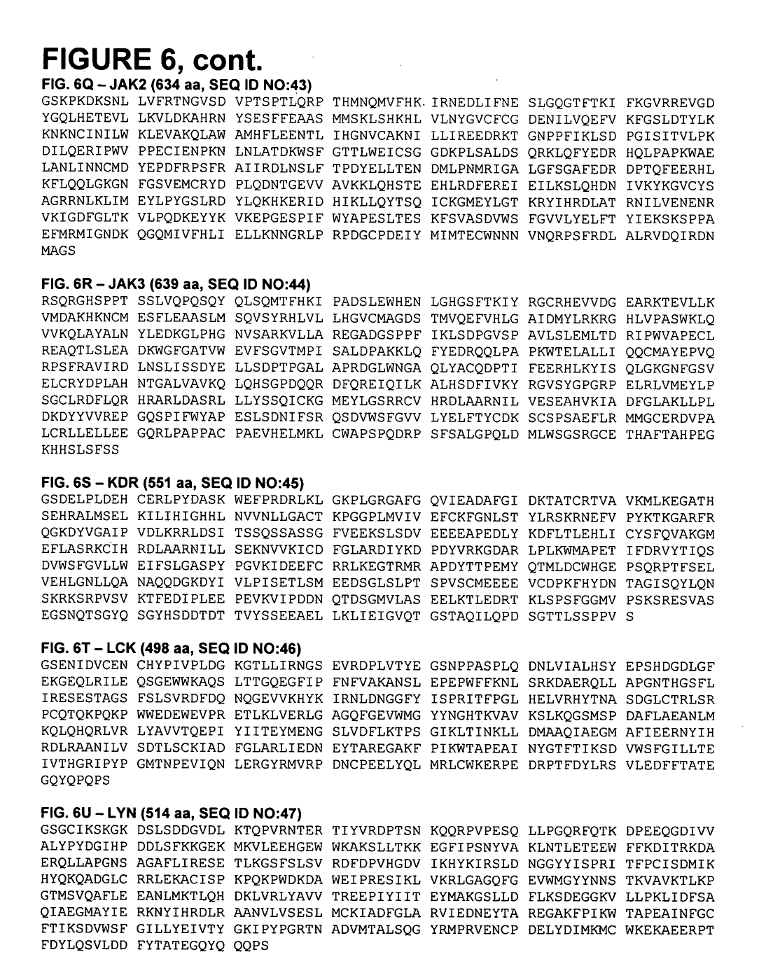

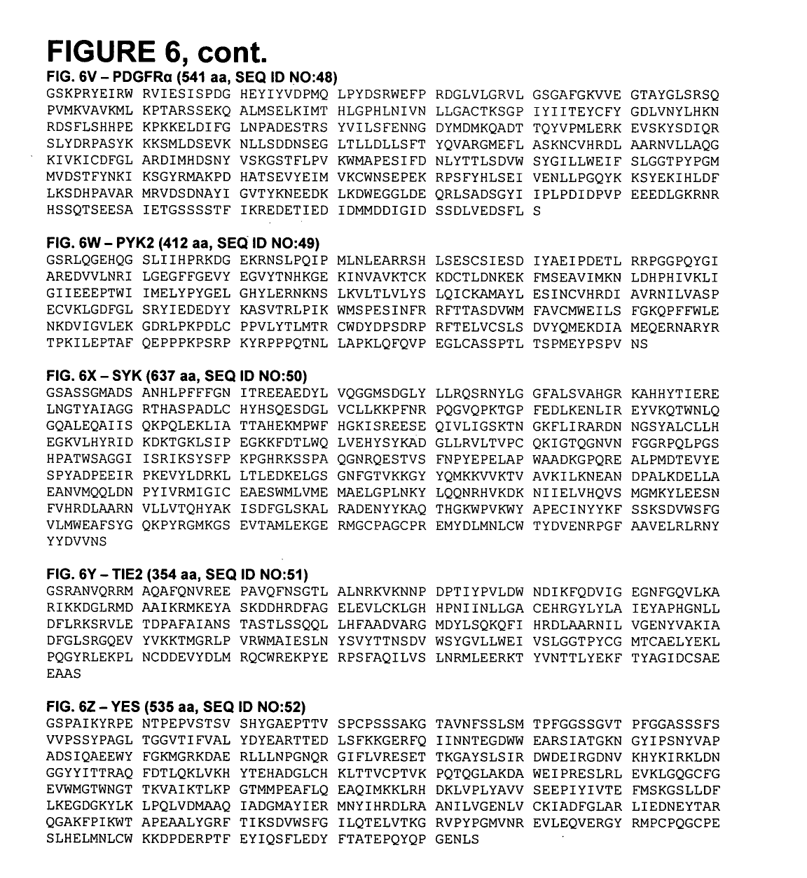

In certain embodiments, the panel of tyrosine kinases contains tyrosine kinases, and/or fragments thereof, selected from EGFR, IGF1R, KIT, VEGFR1, FGFR1, TRKA, MET, EPHB4, AXL, TIE1, DDR1, RET, ROS, ALK, ROR1, MUSK, SRC, ABL, JAK1, ACK1, FAK, FES, BRK, TEC, ZAP70, BLK, BMX, BTK, CSFR, CSK, CTK, DDR2, EPHA2, EPHA4, FGFR2, FGFR4, FGR, FLT3, FRK, FYN, HER2, HER3, JAK2, JAK3, KDR, LCK, LYN, PDGFRα, PYK2, SYK, TIE2, YES and combinations thereof. Exemplary sequences of kinase fragments that can be selected for expression are shown in FIG. 5 and FIG. 6.

In some embodiments, the complete cytoplasmic tails of an RTK beginning right after the transmembrane domain to the C-terminus can be chosen. In other embodiments, CTKs sequences can vary from full-length for smaller kinases (like SRC and BRK) to fragments that contain the kinase domain and, e.g., about 1 to about 200-300 amino acids flanking the kinase domain. Long, multiple domain fragments can be used in the methods provided herein (but can potentially cause problems with the in vitro expression), but can, in some instances, still include the kinase domain in a context of native sequence and/or together with, for example, small, modular SH2 and SH3 domains.

5.1.2 Cloning

Any cloning scheme known in the art can be used in the methods provided herein. The practice of the system and methods provided herein employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields as are within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Ausubel et al., Current Protocols in Molecular Biology, John Wiley & Sons (1987 and annual updates through present); Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) Genome Analysis: A Laboratory Manual (1999) Cold Spring Harbor Laboratory Press.

In some embodiments, the sequence encoding a kinase domain (KD) of the target PTK is amplified by PCR using cDNA preparations from the appropriate cellular source as a template. An N-terminal fluorescent tag and/or C-terminal affinity purification tag can optionally be added to the sequence during amplification (see Section 5.1.3 below). The amplified fragments encoding kinase open reading frames (ORFs) can be connected to regulatory sequences, such as SP6 or T7 promoters, translation initiation sites, translation enhancers, optimized 5′- and 3′-untranslated regions (UTR) using PCR overlap extension technique.

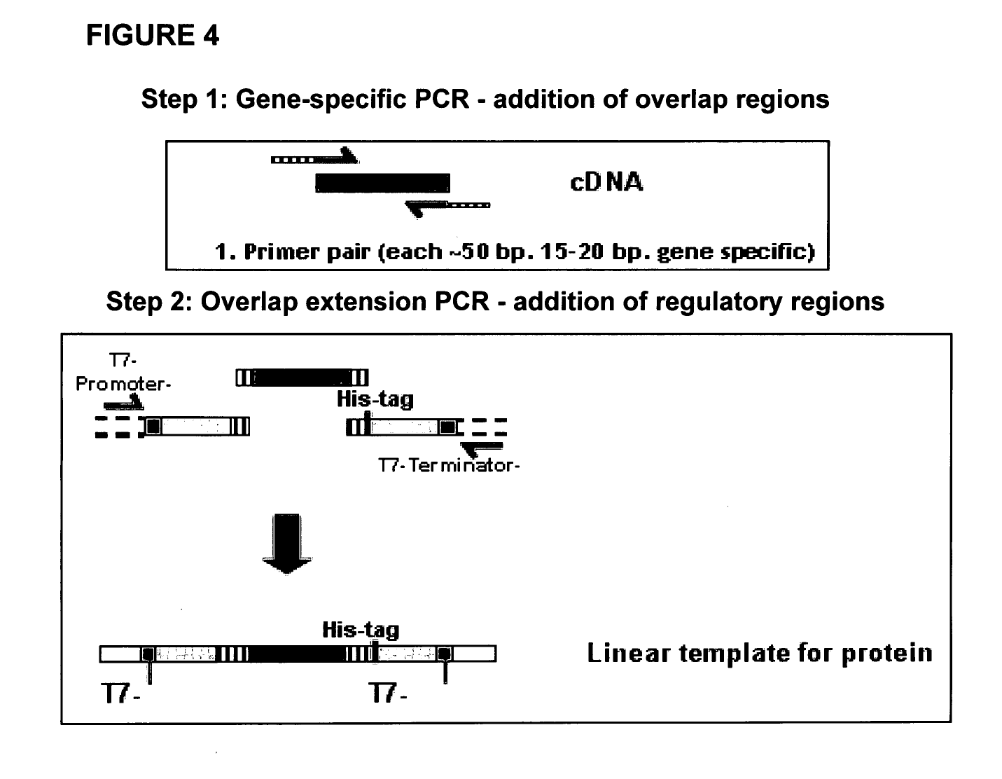

PCR overlap extension is illustrated in FIG. 4 and is a method of creating expression template through PCR. Sequential PCR reactions can be used. For example, in a first reaction, gene-specific primers are used to add overlapping sequences homologous to the “add-on” regions. The product of the first PCR can then be mixed with two flanking primers and DNA fragments coding for the regulatory elements or tag sequences. The product can then anneal with the regulatory/tag fragments and is extended outwards. This final linear expression construct can then be further amplified via the flanking primers. This technique can be used, for example, for the introduction of regulatory regions upstream or tags downstream of the cloned ORF. The linear DNA can then be used directly in IVT reactions.

In one embodiment, a polynucleotide encoding the tyrosine kinase is a linear polynucleotide. In some embodiments, kinase arrays (e.g., a panel of kinases and/or fragments thereof) are produced from PCR DNA in an IVT system. In certain embodiments, the polynucleotide encoding the tyrosine kinase contains regulatory elements, such as SP6 or T7 promoters, translation initiation sites, translation enhancers, optimized 5′ and 3′-UTR.

Various transcription and translation enhancing elements can be introduced in the expressing construct to improve yield of the expressed kinase. These can include an optimized translation initiation site as well as 5′- and 3′-UTR. In the case of a eukaryotic expression system, such as RRL, an optimized translation initiation site can be represented by the starting ATG codon in the context of idealized Kozak sequence, such as GCCGCC(A/G)CC ATG G (Kozak (1987) Nucleic Acids Res. 15(20): 8125-8148). A number of 5′- and 3′-UTRs, including those containing specific translation enhancing sequences, from efficiently translated mammalian and viral genes, for example human globin, bovine growth hormone, barley yellow dwarf virus (Guo et al. (2000) RNA 6(12):1808-20) and encephalomyocarditis virus (Parks et al. (1986) J. Virol. 60:376) coat proteins and many others have been used to enhance expression of target proteins in cell-free systems. Structural features of the 5′ UTR have a major role in the control of mRNA translation. Synthetic “idealized” 5′-UTRs have been also successfully used to improve expression levels of heterologous proteins in vitro (Zozulya et al. (1990) Protein Eng. 3(5):453-8).

See, for example, He et al., (2003) J. Immunol. Meth. 274:265, which is incorporated herein by reference, for general methods applicable to the generation of a PCR construct (and/or in vitro translation methods) suitable for use in the methods provided herein.

5.1.3 Design of Expression Cassettes

In some embodiments, the expression cassettes for IVT optionally include detectable (e.g., fluorescent) and/or affinity purification tags on the N- and/or C-termini of the kinase domain fragment.

Any type of tag may be used with the methods provided herein. The tag can be any type of moiety that is attached to, e.g., a polynucleotide that encodes a kinase, and/or fragment thereof. For example, a polynucleotide that encodes a PTK can contain one or more additional tag-encoding sequences that encode a, e.g., a detectable moiety or a moiety that may aid in affinity purification. When translated, the tag and the PTK protein can be in the form of a fusion protein. In certain embodiments, the detectable tag is a fluorescent tag on the N-terminus, the affinity purification tag is a strep-tag on the C-terminus, or a combination thereof. However, any type of tag may be located on either or both of the N-terminus or C-terminus.

Non-limiting protein tags that can be translationally fused to the N- or C-termini (or both) of the protein of interest to facilitate its expression, detection, purification and/or multimerization (in addition those mentioned elsewhere herein) include beta-galactosidase (lacZ) (Casadaban et al. (1980) J. Bacteriol. 143:971-80), glutathione-S-transferase (GST) (Smith and Johnson (1988) Gene 31-40), staphylococcal protein A (Uhlen et al. (1983) Gene 23:369-378), staphylococcal protein G, dihydrofolate reductase (DHFR), cellulose-binding domains (CBP), galactose-binding protein, calmodulin-binding protein (CBP), maltose-binding protein (malE, pMAL vectors, New England Biolabs) (diGuan (1988) Gene 67:21-30), NusA, ubiquitin, lac repressor, T4 gp55, growth hormone, thioredoxin (TrxA) (LaVallie et al. (1993) Biotechnology 11:187-93), His-patch thioredoxin, THIOFUSION™ system (Invitrogen, Carlsbad, Calif.), chitin-binding domain/intein (IMPACT™ vectors, New England Biolabs, Beverly, Mass.), biotinylated birA substrate mimic peptide tags (Schatz (1993) BioTechnology 11:1138-1143), calmodulin-binding peptide, chloramphenicol acetyltransferase (CAT), TrpE, aviden/treptavidin/Strep tag, T7gene10, OmpT/OmpA/PelB/DsbA/DsbC, KSI, any fluorescent protein, e.g., green fluorescent protein (GFP) (Chalfie et al. (1994) Science 263:802-803 or red fluorescent protein (dsRed) (Baird et al. (2000) Proc. Natl. Acad. Sci. USA 97:11984-11989), or a combination thereof. Any short (typically 6-12 amino acid residues) peptide epitope tags known in the art may also be used either alone or in combination with the other tags described herein, including but not limited to His-tag, influenza virus haemagglutinin (HA), myc, c-myc, FLAG™ peptide (DYKDDDDK (SEQ ID NO:53)), HSV-tag, T7-tag, VSV-G, B-tag (VP7 protein region of bluetongue virus), E-tag, S-tag (Kim and Raines (1993) Protein Sci. 2:348-356), V5, polyarginine (e.g., 5-15 amino acids), polycysteine (e.g., 11 amino acids), polyphenylalanine (e.g., 11 amino acids), (Ala-Trp-Trp-Pro)ₙ, polyaspartic acid (e.g., 5-16 amino acids), or a combination thereof. For descriptions of the above-identified tags and others suitable for use in the methods provided herein, including protein tagging strategies, see Jarvik and Telmer (1998) Annu. Rev. Genet. 32: 601-618; Stevens (2000) Structure 8:R177-R185; Nilsson et al. (1997) Prot. Expression and Purification 11:1-16, which are each incorporated by reference in their entirety.

5.1.3.1 Detectable Tags

In certain embodiments, a detectable tag, such as a fluorescent tag, is on the N-terminal end of the protein. A non-limiting example of a fluorescent tag that may be used in the methods provided herein is the LUMIO™ tag (Invitrogen Corp.). The LUMIO™ tag is a small, six-amino acid sequence Cys-Cys-Pro-Gly-Cys-Cys (SEQ ID NO:54) which can be fused either to the N- or C-terminus of a recombinant protein. The tetracysteine LUMIO™ sequence binds to the corresponding biarsenical LUMIO™ detection dyes with high affinity and specificity resulting in a bright fluorescent signal which can be easily detected and quantified at nanogram protein levels using a standard fluorometric plate reader. LUMIO™ detection dyes are not fluorescent until bound to the tetracysteine recognition sequence. Advantages of the LUMIO™ technology over such alternative protein quantification techniques applicable to the IVT systems as conventional protein assays (Bradford, Folin, etc.), immunodetection of epitope tags or incorporation of radioactively labeled amino acids include sensitivity, convenience, cost, time and labor involved.

Advantages of translational LUMIO™ tag fusions include the small size of the LUMIO™ tag (6 amino acids, 585 Da) which is less likely to interfere with the folding and enzymatic activity of a fused PTK. With LUMIO™ tag, there is no requirement for protein folding and maturation which can compromise functional performance of some conventional fluorescent proteins in cell-free translation systems. In addition, LUMIO™ tag fusions afford the flexibility of using either green (excitation maximum wavelength 508 nm, emission 528 nm) or red (excitation maximum 593 nm, emission 608 nm) LUMIO™ dye for recombinant protein detection or quantification to avoid possible interference with other fluorescent components during high throughput biochemical kinase inhibition assays.

Any fluorescent tag may be used in the methods provided herein. In addition to the LUMIO™ tag, other fluorescent tags that can be used include various fused fluorescent proteins, such as green fluorescent protein (GFP) or in vitro incorporated fluorescent amino acid derivatives (FLUOROTECT™ Green In Vitro Translation Labeling System; Promega Corp.).

5.1.3.2 Affinity Purification Tags

In other embodiments, a tag, such as an affinity tag, is on the C-terminal end of the recombinant protein. A non-limiting example of an affinity purification tag that can be used is the Strep-Tag II sequence (Skerra and Schmidt (2000) Meth. Enzymol. 326:271-304). The Strep-Tag II is a small, 8-amino acid sequence Trp-Ser-His-Pro-Gln-Phe-Glu-Lys (SEQ ID NO:55) that has moderately high affinity (Kd=18 μM) to streptavidin and several-fold better affinity to the engineered streptavidin version called STREPTACTIN™ (IBA GmbH, Germany) when fused to the C- or N-termini of a recombinant protein. This tag can be used for affinity purification of recombinant proteins in mild, native conditions through binding to immobilized streptavidin or STREPTACTIN™ followed by elution by 1-2 mM of biotin or desthiobiotin (Skerra and Schmidt (2000) Meth. Enzymol. 326:271-304).

Any affinity purification tag can be used in the methods provided herein, Other non-limiting examples of affinity purification tags include the higher affinity avidin-binding peptide tags, such as AVITAG™ (Avidity LLC, Denver) or protein-dimerizing affinity purification tags (e.g., a dimerization domain), such as a glutathione S-transferase (GST) tag. A dimerization domain, such as GST can be introduced into the module to serve dual purpose, such as, e.g., for affinity purification of the expressed fusion protein and/or enhancing enzymatic activity of recombinant fusion kinase in a biochemical assay (such as an HTS assay) by facilitating proximity-driven transphosphorylation and activation of kinase domains in the protein dimer. GST-fusions can be used for expressing tyrosine kinases in heterologous systems.

5.1.4 In Vitro Translation (IVT) Systems

The in vitro synthesis of proteins in cell extracts is a powerful research tool and has been widely used for analytical characterization of gene products for decades (Spirin, A. S. ed. (2002) Cell-Free Translation Systems, Springer Verlag, Berlin-Heidelberg-New York; Swartz, J. A. ed. (2003) Cell-Free Protein Expression. Springer Verlag, Berlin-Heidelberg-New York). Unfortunately, typical yields of recombinant proteins in standard RRL system run on the 1 ml scale, for example, are in the range of just a few micrograms, which complicates the use of this system for many research applications and assays. However, this low productivity can be alleviated by the scalability of synthesis reactions, typically without any reduction in translation efficiency, as well as a number of incremental improvements in both the efficiency and cost of the in vitro transcription and translation techniques made over the last decade or so (Gurevich et al. (1991) Anal. Biochem., 195: 207-213; Kigawa et al. (1999) FEBS Letters, 442: 15-19; Madin et al. (2000) Proc. Natl. Acad. Sci. USA, 97(2): 559-564; Sawasaki et al. (2002) Proc. Natl. Acad. Sci. USA, 99(23): 14652-14657). Also, typical reported yields are several-fold higher in WGE system, as compared to RRL, and could be much higher in E. coli cell-free system, sometimes approaching hundreds of micrograms of protein per 1 ml of IVT reaction.

Recently, “continuous” in vitro translation systems have been developed which allows for the production of hundreds of micrograms to milligrams of recombinant protein from linear, PCR-generated templates in hours or days (Spirin et al. (1988) Science, 242:1162-64) and commercialization by Roche (Betton (2003) Curr. Protein Pept. Sci., 4(1):73-80). Several additional versions of these continuous-flow cell-free (CFCF) and continuous-exchange cell-free (CECF) high-productivity IVT systems have also recently been developed (Endo (2002) FEBS Letters, 514: 102-105; Endo and Sawasaki (2003) Biotechnology Advances, 21: 695-713; Spirin, A. S. ed. (2002) Cell-Free Translation Systems, Springer Verlag, Berlin-Heidelberg-New York; Swartz, J. A. ed. (2003) Cell-Free Protein Expression. Springer Verlag, Berlin-Heidelberg-New York).

In some embodiments, the in vitro translation system is a WGE, RRL, or S30 cell-free translation system, or a combination thereof. However, any IVT system can be used in the methods provided herein. For example, active cell-free systems have been obtained from such sources as yeast (Tuite et al. (1980) J. Biol. Chem. 255:8761), and HeLa cells (Gallwitz et al. (1978) Meth. Cell Biol. 19:197-213, among others. WGE, RRL, and S30 can be prepared as crude extracts containing all the macromolecular components (e.g., 70S or 80S ribosomes, tRNAs, aminoacyl-tRNA synthetases, initiation, elongation and termination factors, etc.) required for translation of exogenous RNA. To ensure efficient translation, each extract can be supplemented with amino acids, energy sources (e.g., ATP, GTP), energy regenerating systems (e.g., creatine phosphate and creatine phosphokinase for eukaryotic systems, and phosphoenol pyruvate and pyruvate kinase for the E. coli lysate), and other co-factors (e.g., Mg²⁺, K⁺, etc.).

5.1.4.1 “Linked” And “Coupled” Transcription:Translation Systems

At least two approaches to in vitro protein synthesis can be used in the methods provided herein based on the starting genetic material (e.g., RNA or DNA). Standard translation systems, such as RRL and WGE, use RNA as a template; whereas “coupled” and “linked” systems start with DNA templates, which are transcribed into RNA then translated. The most popular cell-free translation systems consist of lysates or extracts from rabbit reticulocytes, wheat germ and E. coli cells (e.g., S30 system). All are prepared from the corresponding source cells as crude extracts containing all the macromolecular components, amino acids, energy sources, energy regenerating systems, and various co-factors required for translation of added RNA in a test-tube.

“Linked” and “coupled” systems use DNA as a template, as compared to standard translation reactions, in which purified RNA is used. RNA is transcribed from the DNA and subsequently translated without any purification. Such systems typically combine a prokaryotic phage RNA polymerase and promoter (e.g., T7, T3, or SP6) with eukaryotic or prokaryotic extracts to synthesize proteins from exogenous DNA templates. DNA templates for transcription:translation reactions may be cloned into plasmid vectors or generated by PCR.

Coupled Transcription:Translation. Unlike eukaryotic systems where transcription and translation occur sequentially, in E. coli, transcription and translation occur simultaneously within the cell. In vitro E. coli S30 translation systems are thus performed the same way, coupled, in the same tube under the same reaction conditions (one-step reaction). During transcription, the 5′ end of the RNA becomes available for ribosomal binding and undergoes translation while its 3′ end is still being transcribed. This early binding of ribosomes to the RNA maintains transcript stability and promotes efficient translation. This bacterial translation system gives efficient expression of either prokaryotic or eukaryotic gene products in a short amount of time. For the highest protein yield and the best initiation fidelity, the DNA template can have a Shine-Dalgamo ribosome binding site upstream of the initiator codon. Capping of eukaryotic RNA may be done, but is not required. Use of E. coli extract also eliminates cross-reactivity or other problems associated with endogenous proteins in eukaryotic lysates. Also, the E. coli S30 extract system allows expression from DNA vectors containing natural E. coli promoter sequences (such as lac or tac).

A coupled transcription:translation system can be used in the methods provided herein. A non-limiting example of a coupled system that may be used is the TNT® Quick Coupled Transcription/Translation Systems (Promega). In certain embodiments, a promoter-containing plasmid (e.g., a T7- or SP6-containing plasmid) is added to a tube containing an in vitro translation extract (e.g., RRL or WGE) along with the appropriate polymerase (e.g., T7 or SP6 RNA polymerase), nucleotide triphosphates, and magnesium (Mg²+) and other required factors. Non-limiting examples of coupled transcription:translation reactions that may be used in the methods provided herein are described in U.S. Pat. Nos. 5,492,817; 5,665,563; and 5,324,637.

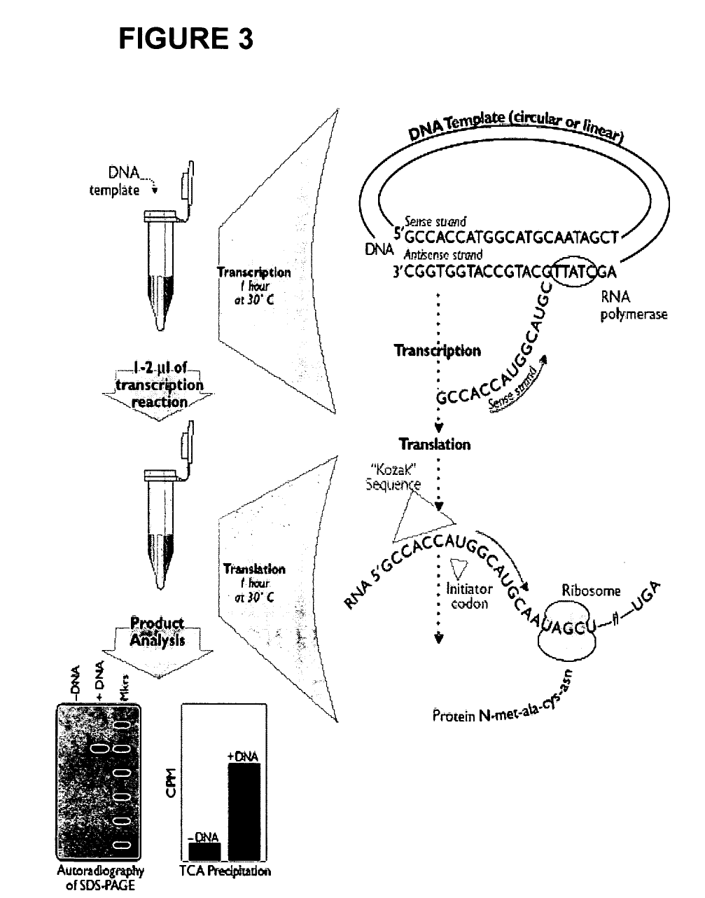

Linked Transcription:Translation. The “linked” system is a two-step reaction, based on transcription with a bacteriophage-promoter specific polymerase (e.g., SP6, T7 or T3) followed by translation in the RRL or WGE (FIG. 3). Because the transcription and translation reactions are done in separate tubes, each can be optimized to ensure that both are functioning at their full potential. Conversely, many commercially available eukaryotic coupled transcription:translation systems have compromised one or both reactions so that they can occur in a single tube, and yield may be sacrificed for convenience.

Both linked and coupled systems can be used in the methods provided herein. DNA templates for transcription-translation reactions can be either cloned into plasmid vectors or generated by PCR as a linear fragment containing phage promoter.

5.1.4.2 Rabbit Reticulocyte Lysate

RRL is a highly efficient in vitro eukaryotic protein synthesis system used for translation of exogenous RNAs (either natural or generated in vitro). In vivo, reticulocytes are highly specialized cells primarily responsible for the synthesis of hemoglobin, which represents more than 90% of the protein made in the reticulocyte. These immature red cells have already lost their nuclei, but contain adequate mRNA, as well as complete translation machinery, for extensive globin synthesis. The endogenous globin mRNA can be eliminated by incubation with Ca²⁺-dependent micrococcal nuclease, which is later inactivated by chelation of the Ca²⁺ by EGTA. A nuclease-treated RRL may also be used (Ambion). Untreated RRL translates endogenous globin mRNA, exogenous RNAs, or both. This type of lysate is typically used for studying the translation machinery, e.g., studying the effects of inhibitors on globin translation. Both the untreated and treated RRL have low nuclease activity and are capable of synthesizing a large amount of full-length product. Both lysates are appropriate for the synthesis of larger proteins from either capped or uncapped RNAs (eukaryotic or viral).

5.1.4.3 Wheat Germ Extract

WGE is a robust and widely used eukaryotic expression system which can provide much higher protein yields than other IVT systems, such as the RRL system. Recent improvements in the WGE system are disclosed in Sawasaki et al. (2002) Proc. Natl. Acad. Sci. USA, 99(23): 14652-14657 and Sawasaki et al. (2002a) FEBS Letters, 514:102-105, and can be used with the methods provided herein.

WGE is a convenient alternative to the RRL cell-free system. This extract has low background incorporation due to its low level of endogenous mRNA. WGE efficiently translates exogenous RNA from a variety of different organisms, from viruses and yeast to higher plants and mammals. WGE is recommended for translation of RNA containing small fragments of double-stranded RNA or oxidized thiols, which are inhibitory to the RRL. Both RRL and WGE translate RNA isolated from cells and tissue or those generated by in vitro transcription. When using RNA synthesized in vitro, the presence of a 5′ cap structure may enhance translational activity. Typically, translation by WGE is more cap-dependent than translation by RRL. If capping of the RNA is impossible and the protein yield from an uncapped mRNA is low, the coding sequence can be subcloned into a prokaryotic vector and expressed directly from a DNA template in an E. coli S30 cell-free system. However, in certain embodiments, the polynucleotides encoding the kinase, kinase domain, and/or fragment thereof, is a linear polynucleotide.

5.1.4.4 E. coli Cell-Free System

The E. coli S30 system can also be used in the methods provided herein. The advantages of this system include the highest reported protein yields for in vitro translation, as well as its commercialization as a part of high-productivity CEFE RTS (Rapid Translation System; Roche Applied Science). When the S30 IVT system is used, kinase templates can be re-engineered by PCR to include prokaryotic regulatory elements, such as a bacteriophage promoter (e.g., SP6, T7 or T3) and efficient prokaryotic ribosome-binding site (RBS), also called Shine-Dalgarno sequence. This purine-rich sequence of 5′ UTR is complementary to the UCCU core sequence of the 3′-end of 16S rRNA (located within the 30S small ribosomal subunit). These sequences lie about 10 nucleotides upstream from the AUG start codon. Activity of a RBS can be also influenced by the length and nucleotide composition of the spacer separating the RBS and the initiator AUG. Structure of 5′- and 3′-untranslated regions can also affect translation efficiency in prokaryotic systems. Moderately long 5′-UTR with minimal secondary structure are typically optimal for translation.

Due to the in vitro nature of translation, the S30 IVT system can also optionally be supplied by any needed chaperonins or co-factors either in purified form or in form of cellular extracts containing them (see, e.g., Xu et al. (2002) Mol. Cell Biol., 22: 4419-4432; Yokoyama et al. (2003) Curr. Opin. Chem. Biol., 7:39-43).

E. coli cell-free systems can consist of a crude extract that is rich in endogenous mRNA. The extract can be incubated during preparation so that this endogenous mRNA is translated and subsequently degraded. Because the levels of endogenous mRNA in the prepared lysate is low, the exogenous product can be easily identified. In comparison to eukaryotic systems, the E. coli extract has a relatively simple translational apparatus with less complicated control at the initiation level, allowing this system to be very efficient in protein synthesis. Bacterial extracts are often unsuitable for translation of RNA, because exogenous RNA is rapidly degraded by endogenous nucleases. There are some viral mRNAs (TMV, STNV, and MS2) that translate efficiently, because they are somewhat resistant to nuclease activity and contain stable secondary structure. However, E. coli extracts are ideal for coupled transcription:translation from DNA templates.

The in vitro synthesis of proteins in cell extracts is a powerful research tool and has been widely used for analytical characterization of gene products for decades (Spirin, A. S. ed. (2002) Cell-Free Translation Systems, Springer Verlag, Berlin-Heidelberg-New York; Swartz, J. A. ed. (2003) Cell-Free Protein Expressionr. Springer Verlag, Berlin-Heidelberg-New York). Advantages of the IVT kinase-expression systems and methods provided herein over in vivo gene expression include extreme simplicity, speed and ease of use as well as minimal requirements for specialized lab equipment and reagents, as illustrated in FIG. 3. A typical sequence of in vitro transcription and translation reactions, whether coupled or separate, and isolation (e.g., affinity purification or immunoprecipitation) of the synthesized protein can be done in one working day, and multiple samples can be easily processed simultaneously or in sequence. This compares very favorably with any cell-based alternative. In addition, since most IVT systems accept small amounts of linear DNA fragments generated by PCR as templates for transcription or coupled, single-tube transcription/translation, cloning in E. coli can be also eliminated. In other words, addition of a kinase or a mutant kinase variant to a biochemical assay panel can be implemented as a completely cell-free, two-day protocol carried out in a few test tubes.

Additionally, the IVT kinase-expression systems and methods provided herein can be used for the screening of test compounds, including small-molecules, in a “quasi in vivo” format. This can be done, for example, by adding a tested compound directly to the IVT reaction prior to initiating translation. This “co-translational” format will allow the detection of a kinase modulatory (e.g., inhibitory) effect unrelated to the direct inhibition of a catalytic event, such as interference with proper kinase folding, as well as eliminating some toxic compounds, for example those inhibiting cellular translation apparatus, prior to cellular assays.

In specific embodiments, the IVT system is WGE, RRL, and/or S30. In some embodiments, the panel of tyrosine kinases contains about 5, 10, 15, 20, 30, 40, 50, 60, 70, 80, 90 or more, or such as about 100, 150, 200, 250, 300 350, 400, 450, 500 or more tyrosine kinases, including the same or different families or subfamilies of tyrosine kinases, the same or different forms (e.g., wild-type or mutant) of tyrosine kinases, and/or fragments thereof, expressed using a WGE, RRL and/or S30 IVT system, wherein said panel contains tyrosine kinases, and/or fragments thereof, that are human non-receptor tyrosine kinases, human receptor tyrosine kinases, or a combination thereof, and wherein substantially all of the kinases, and/or fragments thereof, have kinase activity. In certain embodiments, the panel of tyrosine kinases contains tyrosine kinases, and/or fragments thereof, selected from EGFR, IGF1R, KIT, VEGFR1, FGFR1, TRKA, MET, EPHB4, AXL, TIE1, DDR1, RET, ROS, ALK, ROR1, MUSK, SRC, ABL, JAK1, ACK1, FAK, FES, BRK, TEC, ZAP70, BLK, BMX, BTK, CSFR, CSK, CTK, DDR2, EPHA2, EPHA4, FGFR2, FGFR4, FGR, FLT3, FRK, FYN, HER2, HER3, JAK2, JAK3, KDR, LCK, LYN, PDGFRα, PYK2, SYK, TIE2, YES and combinations thereof.

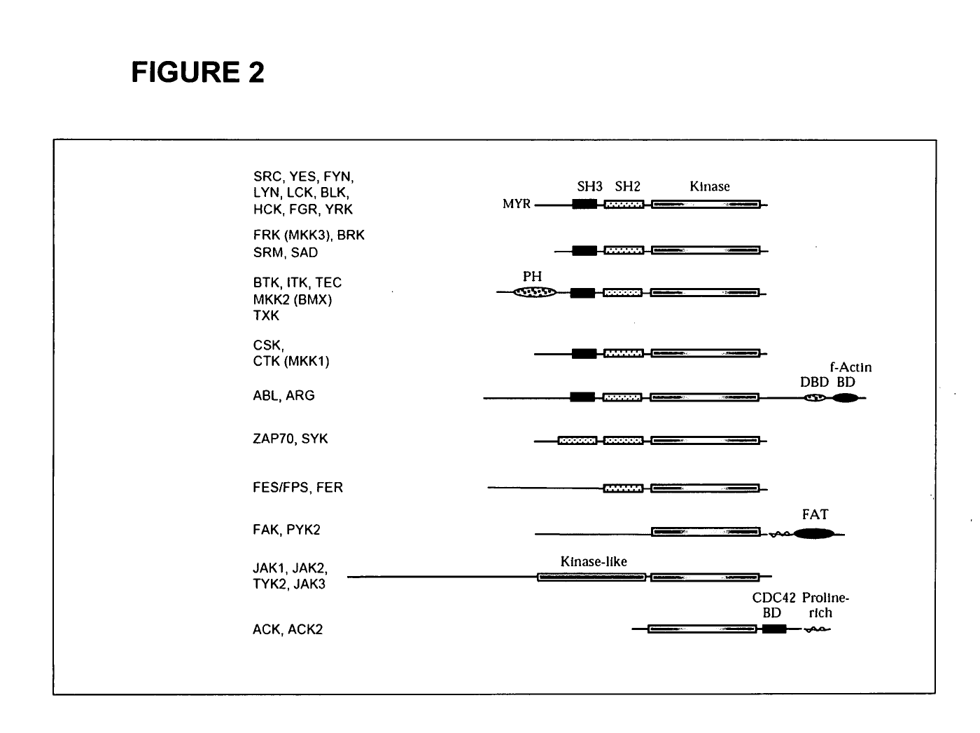

In certain embodiments, the panel of tyrosine kinases contains about 5, 10, 15, 20, 30, 40, 50, 60, 70, 80, 90 or more, or such as about 100, 150, 200, 250, 300 350, 400, 450, 500 or more tyrosine kinases, including the same or different families or subfamilies of tyrosine kinases, the same or different forms (e.g., wild-type or mutant) of tyrosine kinases, and/or fragments thereof, expressed using a WGE, RRL and/or S30 IVT system, wherein said panel contains tyrosine kinases representing all major, therapeutically attractive subfamilies of the tyrosine kinase family (see, e.g., FIG. 1 and FIG. 2). In certain embodiments, the panel of tyrosine kinases are expressed using a WGE system and contain tyrosine kinases, and/or fragments thereof, selected from EGFR, IGF1R, KIT, VEGFR1, FGFR1, TRKA, MET, EphB4, AXL, TIE1, DDR1, RET, ROS, ALK, ROR1, Musk, SRC, ABL, JAK1, ACK1, FAK, FES, BRK, TEC, ZAP70, BLK, BMX, BTK, CSFR, CSK, CTK, DDR2, EPHA2, EPHA4, FGFR2, FGFR4, FGR, FLT3, FRK, FYN, HER2, HER3, JAK2, JAK3, KDR, LCK, LYN, PDGFRα, PYK2, SYK, TIE2, YES and combinations thereof. In certain embodiments, WGE, RRL and/or S30 IVT is used to express a panel of tyrosine kinase domains, such as those depicted in FIG. 5 and FIG. 6.

In some embodiments, functional kinase domains of selected members of at least one major human protein tyrosine kinase family and/or subfamily are expressed in an IVT system, such as WGE, RRL and/or S30 cell-free translation systems. In certain embodiments, functional kinase domains of selected members of about 5 or more, about 10 or more, or all major human protein tyrosine kinase families and/or subfamilies are expressed in an IVT system, such as WGE, RRL and/or S30 cell-free translation systems, in amounts suitable for HTS and kinase inhibitor specificity profiling.

5.1.5 Protein Purification

The purification of the panel kinases can be mediated by the affinity purification tag, such as a C-terminally located Strep-tag, while the quantification of the protein can be based on the presence of the detectable tag, such as an N-terminal LUMIO™ fluorescent tag. This arrangement of tags can ensure quantification of only the affinity-purified full-length protein. For example, the Step-tag located on the C-terminal end of the protein can ensure that proteins that have been prematurely translated will be eliminated during the affinity purification step. Additionally, any rare N-terminally truncated contaminants, e.g., those derived by proteolytic cleavage, will not be quantified due to the lack of fluorescent tag on their N-termini. In certain embodiments, kinases in the panel are purified in parallel (e.g., simultaneously). In other embodiments, the kinases in the panel are not purified in parallel.

5.1.6 Monitoring Kinase Expression

In some embodiments, kinase expression is monitored. Expression of each kinase can be monitored by any method known in the art. In some embodiments, sodium dodecyl sulfate polyacrylamide gel electrophoresis (SDS-PAGE) followed by direct in-gel fluorescent detection based on the presence of a fluorescent tag (e.g., LUMIO™ tag) is used to monitor kinase translation and expression. In other embodiments, Western blotting followed by detection with streptavidin-enzyme conjugates is used to monitor kinase translation and expression. Comparative levels of expression and integrity of the expressed proteins can be evaluated at this step and necessary adjustments, such as optimization of regulatory elements in the template can be done.

5.1.7 Quantitation of Kinase Expression

In some embodiments, the yield of translation reaction is determined. Quantitation of the expressed proteins can be done using any method known in the art. In some embodiments, quantitation of expressed proteins is done fluorimetrically taking advantage of the fluorescent tag (e.g., a red or green LUMIO™ tag). Since the bi-arsenical LUMIO™ dyes fluoresce only when they bind to their recognition sequence on the recombinant protein, these measurements can be made both with and without purifying the expressed kinase after translation. To determine absolute molar amount of each kinase, the fluorimetric measurements can be done in comparison with serial dilutions of a control fluorescent-tagged (e.g., LUMIO™-tagged) protein, such as an expression construct that is known to express high levels of an easily purifiable protein (e.g., a PTK) in a cell-based system. For many assay applications, exact quantitation of active kinase may not be necessary as long as kinase activity is reliably detected in the assay with a sufficiently high signal-to-noise ratio.

5.1.8 Confirmation of Kinase Activity

In some embodiments, kinase activity of the expressed tyrosine kinases, and/or fragments thereof, is determined. Kinase activity can be determined and/or measured using any method(s) known in the art. For example, in some embodiments, the autophosphorylation status of the expressed tyrosine kinases, and/or fragments thereof, as well as transphosphorylation of protein substrates present in translation mixtures, can be checked, for example, by Western blotting of IVT reactions and purified translation products with anti-phosphotyrosine antibodies. Endogenous levels of tyrosine phosphorylation in WGE, RRL or S30 systems are negligible or nonexistent making such detection trivial. In other embodiments, kinase activities of the expressed kinases is determined by a standard in vitro kinase assay after purification of a translation product, such as by using an affinity tag or by immunoprecipitating it with a kinase-specific antibody. For example, purified kinase can be supplied with a kinase reaction buffer containing ATP and one of the commercially available general or kinase-specific substrate, such as poly(Glu-Tyr), enolase or synthetic peptide substrates. Progress of in vitro reactions can be evaluated by Western anti-P-Tyr blots or incorporation of radiolabel. In yet other embodiments, the kinase is tested in one or several biochemical HTS assay formats, such as a fluorescence- or luminescence-based commercial technologies (e.g., ALPHASCREEN™ (Perkin Elmer), KINASE-GLO™ (Promega, Madison, Wis.) and/or KINOME HUNTER® (DiscoveRx, Fremont, Calif.)).

In addition to the kinase techniques mentioned above, which are known in the art, compilations of detailed kinase protocols can be found in Reith, A. D. (Ed.), Methods in Molecular Biology, v.124. Protein Kinase Protocols, Totowa, N.J., Humana Press, 2001 and/or Hunter, T. (Ed.), Methods in Enzymology, v. 200, Protein Phosphorylation, Part A: Protein Kinases: Assays, Purification, Antibodies, Functional Analysis, Cloning, and Expression, Academic Press, 1991.

In addition, one of a variety of commercial kinase assays may also be used. Non-limiting examples of commercial assays that can be used include IQ® Assay (Pierce Biotechnology Corp., Rockford, Ill.) and Z′-LYTE™ (Invitrogen Corp., Carlsbad, Calif.).

5.2 Methods of Screening for a Modulator of Tyrosine Kinase Activity

Also provided herein are methods of screening for a modulator of tyrosine kinase activity (e.g., the activity of one or more PTK, and/or fragments thereof), said method involving:
providing one or more polynucleotides that encode one or more polypeptides containing a tyrosine kinase domain, and/or fragment thereof,
optionally adding a first tag (e.g., a fluorescent tag) to the N-terminus of one or more of the polynucleotides,
optionally adding a second tag (e.g., an affinity purification tag) to the C-terminus of one or more of the polynucleotides,
translating the one or more polynucleotides in an in vitro cell-free translation (IVT) system, wherein the resulting one or more polypeptides contain a tyrosine kinase domain, and/or fragment thereof, having kinase activity,
contacting a test compound with the one or more polypeptides, and
detecting modulation of kinase activity the one or more polypeptides relative to kinase activity in the absence of test compound.
   In specific embodiments, the one or more polynucleotides encode a panel of PTK (e.g., RTK and/or CTK), and/or fragments thereof (e.g., kinase domains and/or active fragments thereof), wherein substantially all of said PTKs in the panel have kinase activity. In certain embodiments, the one or more (such as about 5, 10, 15, 20, 30, 40, 50, 60, 70, 80, 90 or more, or such as about 100, 150, 200, 250, 300 350, 400, 450, 500 or more) polynucleotides encode for the same or different tyrosine kinases (e.g., PTK, RTK and/or CTK), and/or fragments thereof. In some embodiments, the one or more polynucleotides encode for tyrosine kinases, and/or fragments thereof, from different families and/or subfamilies of tyrosine kinases (see, e.g., FIG. 1, Manning et al. (2002) Science 298:1912)). In another embodiment, the one or more polynucleotides encode for tyrosine kinases, and/or fragments thereof, from the same family and/or subfamily of tyrosine kinases. In an embodiment, the one or more polynucleotides encode for tyrosine kinases, and/or fragments thereof, wherein the tyrosine kinases, and/or fragments thereof, are in different forms (e.g., wild-type and/or mutant forms) of the same kinase, different forms of different kinases, or combinations thereof. In a specific embodiment, the one or more polynucleotides encode a tyrosine kinase domain (or an active fragment thereof).

In certain embodiments, the panel of tyrosine kinases contains about 5, 10, 15, 20, 30, 40, 50, 60, 70, 80, 90 or more, or such as about 100, 150, 200, 250, 300 350, 400, 450, 500 or more tyrosine kinases, including the same or different families or subfamilies of tyrosine kinases, the same or different forms (e.g., wild-type or mutant) of tyrosine kinases, and/or fragments thereof, that are human non-receptor tyrosine kinases, human receptor tyrosine kinases, or a combination thereof (see Section 5.1.1 above). In certain embodiments, the panel of tyrosine kinases contains tyrosine kinases, and/or fragments thereof, selected from EGFR, IGF1R, KIT, VEGFR1, FGFR1, TRKA, MET, EPHB4, AXL, TIE1, DDR1, RET, ROS, ALK, ROR1, MUSK, SRC, ABL, JAK1, ACK1, FAK, FES, BRK, TEC, ZAP70, BLK, BMX, BTK, CSFR, CSK, CTK, DDR2, EPHA2, EPHA4, FGFR2, FGFR4, FGR, FLT3, FRK, FYN, HER2, HER3, JAK2, JAK3, KDR, LCK, LYN, PDGFRα, PYK2, SYK, TIE2, YES and combinations thereof.

In one embodiment, a polynucleotide encoding the tyrosine kinase is a linear polynucleotide. In some embodiments, kinase arrays (e.g., a panel of kinases and/or fragments thereof) are produced from PCR DNA in an IVT system. In certain embodiments, the one or more polynucleotides encoding the tyrosine kinases contain regulatory elements (see Sections 5.1.2 and 5.1.3 above). In some embodiments, the in vitro translation system is a WGE, RRL, or S30 cell-free translation system, or a combination thereof (see Section 5.1.5 above).

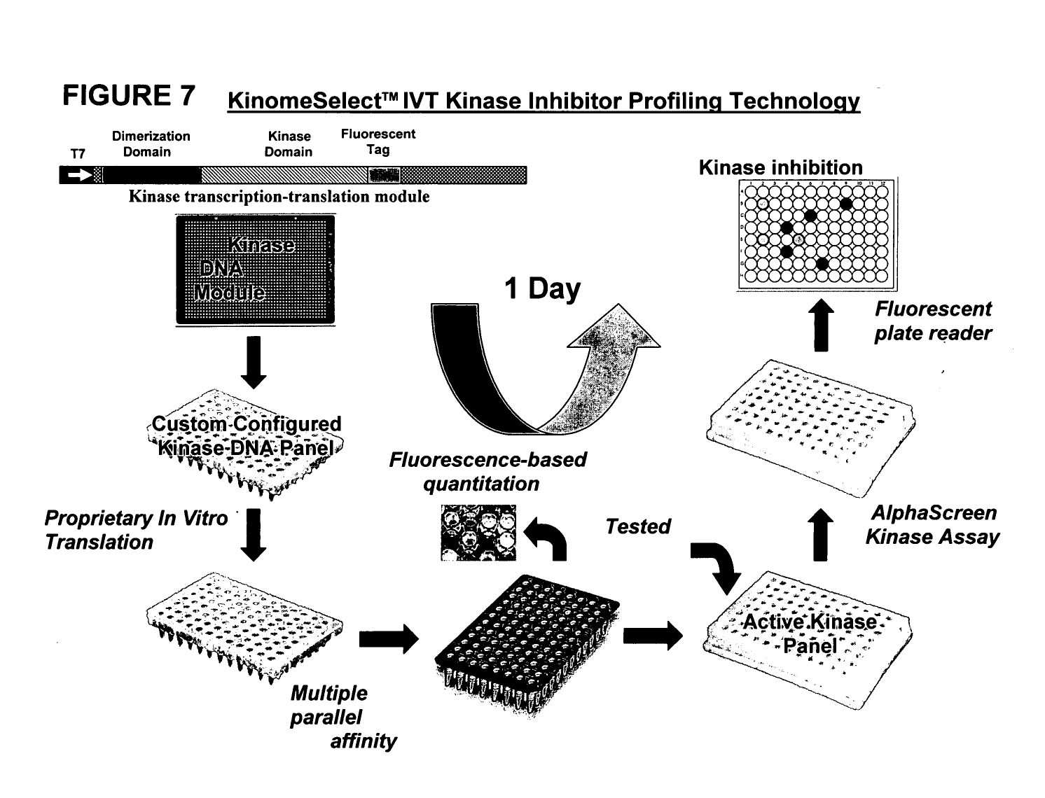

A non-limiting schematic representation of one embodiment is provided in FIG. 7. In certain embodiments, one or more linear polynucleotide (“kinase transcription-translation module”) is created, which contains a T7 promoter (or another bacteriophage promoter, such as SP6 or T3), an optimized translation initiation context (such as 5′-untranslated region (UTR) and Kozak sequence for eukaryotic in vitro translation systems), a dimerization domain, a kinase domain, and a fluorescent tag. The polynucleotides can contain the same or different kinases and/or different forms of kinases, and/or active fragments thereof, multiple naturally occurring kinase mutants, synthetically prepared kinase mutants, structurally comparable forms of one or more native (e.g., wild-type) kinases, and/or functionally comparable forms of one or more native kinases. Each of the linear polynucleotides can be part of the “DNA module bank.” Depending on need, a custom configured kinase panel can be created, and translated in vitro. The kinases can be further affinity purified and quantitated using the respective tags. One or more test compounds can then be added to the custom configured kinase panel, and a routine kinase assay is performed to assess a test compound's kinase inhibition profile. Any or all of the processing steps (e.g., translation, purification, quantitation, etc.) can be completed in a multiple parallel manner in multiwell (e.g., 96-well, 384-well or higher) format and can be easily automated.

Non-limiting examples of test compounds that can be used in the methods provided herein include any protein, polypeptide, peptide, organic molecule, inorganic molecule, antibody, pharmaceutical, and/or candidate pharmaceutical that are natural products or prepared synthetically, and/or any compound found in the U.S. Pharmacopoeia (USP) and/or Physician's Desk Reference (59^{th} ed., 2005; 60^{th} ed., 2006), which are incorporated herein by reference in their entirety.

In certain embodiments, a test compound is screened against a panel (e.g., more than 5, more than 10, more than 25, more than 50, more than 100, more than 150, more than 200, more than 250, more than 300, more than 350, more than 400, more than 450, or more than 500) of different kinases and/or different forms of kinases, and/or active fragments thereof, simultaneously or in sequence. In other embodiments, more than one test compound (e.g., more than 5, more than 10, more than 25, more than 50, or more than 100) is screened against a panel (e.g., more than 5, more than 10, more than 25, more than 50, more than 100, more than 150, more than 200, more than 250, more than 300, more than 350, more than 400, more than 450, or more than 500) of different kinases and/or different forms of kinases, and/or active fragments thereof, simultaneously or in sequence. In some embodiments, the panel contains multiple naturally occurring kinase mutants, synthetically prepared kinase mutants, structurally comparable forms of one or more native (e.g., wild-type) kinases, and/or functionally comparable forms of one or more native kinases. In some embodiments, the kinase panel contains one or more mutant forms of the same kinase, for example multiple mutant forms involved in the manifestation of a given disease (drug “profiling”). In certain embodiments, the screens are completed in a single reaction on a single test plate or a single reaction on multiple test plates (in singlet or duplicate wells). In other embodiments, the screens are performed in multiple reactions on a single test plate or multiple reactions on multiple test plates. In certain embodiments, the screening methods provided herein are high-throughput screens (HTS), e.g., in a 384-well or higher format. Test compounds can be screened against a panel of kinases at a single concentration or at multiple concentrations of the test compound(s) and/or kinase(s). In certain embodiments, the test compounds are screened in parallel. In all embodiments, concentrations, dilution series, replicates, and other assay parameters can be varied. In certain embodiments, simultaneous screens are performed to reduce variability.

In certain embodiments, a panel of PTK (e.g., about 40, about 50, about 60, about 70, about 80 or about 90 or more different PTK) is placed in each well of a 384-well plate at varying concentrations (e.g., 0 μM, 0.2 μM, 1 μM, 5 μM, and 20 μM) so as to quantitatively estimate the inhibitory effect (IC₅₀) of the test compound. In some embodiments, each of the approximately 90 known PTK family members are included in the panel. In one embodiment, a smaller subset of the approximately 90 known PTK family members are included in the panel. In yet other embodiments, various mutant forms known to be involved in a particular cancer, tumor or other malignancy are included in the panel. The mutant forms may or may not be known to have a potential for differential sensitivity to a test compound (e.g., a PTK inhibitor).

In some embodiments, the PTKs expressed in vitro are purified and quantified (e.g., by fluorometric quantification) simultaneously (see Section 5.1 above). In certain embodiments, the PTKs expressed in vitro contain an affinity and/or a fluorescent tag (see Section 5.1.3 above).

In one embodiment, the screening methods provided herein encompass a high-throughput screen (HTS). For example, the methods provided herein encompass HTS in, e.g., a 384-well or higher format. However, any type of commercial assay platform(s) with sufficient sensitivity to detected submicrogram amounts of active kinases (e.g., ALPHASCREEN™ and other shown below) can be used with the IVT-produced panel of kinases and methods provided herein. Non-limiting examples of commercial HTS assays for protein kinases that may be used in the methods provided herein include ALPHASCREEN™ (Perkin Elmer), KINASEGLO™ (Promega, Madison, Wis.) and/or KINOME HUNTER® (DiscoveRx, Fremont, Calif.), IQ® Assay (Pierce Biotechnology Corp., Rockford, Ill.), Z′-Lyte™ (Invitrogen Corp., Carlsbad, Calif.) and TRuLight™ (Calbiochem/EMD Biosciences, Inc.

In an embodiment, the methods provided herein involves contacting one or more test compounds with an IVT-produced kinase in each well and determining if kinase activity of a given IVT-produced kinase is modulated (e.g., increased, decreased, inhibited) relative to the kinase activity in the absence of the test compound or relative to a negative (or positive) control test compound.

The methods provided herein further encompass a kinase inhibitor (or other kinase modulator) selectivity profiling screen (see, e.g., FIG. 7). In some embodiments, the kinase inhibitor selectivity profiling screening methods use the in vitro expressed kinases provided herein, at least one HTS assays discussed elsewhere herein (e.g., ALPHASCREEN™, KINASE-GLO™, KINOME HUNTER®, etc.), and at least one tyrosine kinase inhibitor, with a known inhibition profiles. Non-limiting examples of a tyrosine kinase inhibitor.

In specific embodiments, a HTS approach is used in the methods provided herein. In one embodiment, the HTS is an automated high-throughput screen. In some embodiments, at least one commercial assay platform is used (e.g., ALPHASCREEN™ Amplified Luminescent Proximity Homogeneous Assay; Perkin Elmer). In certain embodiments, the commercial assay platform is implemented on a universal plate reader, such as the Perkin Elmer FUSION™ universal plate reader in, e.g., 384-well or higher format. Any number of test compounds for screening can be selected and used in the methods provided herein. For example, a small set of diverse compounds for screening can be selected from a chemical library, which can include, for example, known PTK inhibitors mentioned above, some kinase inhibitor leads, random control compounds, or a combination thereof. Test compounds that can be used in the IVT systems and methods provided herein include test compounds obtained from small molecule, peptide or protein phage display libraries, alternative display system (e.g., ribosome, yeast, phage, bacterial, antibody) libraries, and/or combinatorial libraries (see, e.g., Moos et al. (1993) Ann. Rep. Med. Chem. 28:315; Pavia et al. (1993) Bioorganic Medicinal Chem. Lett. 3:387; Gallup et al. (1994) J. Med. Chem. 37:1233; Gordon et al. (1994) J. Med. Chem. 37:1385)).

In some embodiments, a test compound can be profiled. In one methods, a test compound is screened in a primary screen against a kinase panel at a given concentration. In a secondary screen, quantitative binding constants can be determined using routine methods for each “hit” identified in the primary screen.

In some embodiments, a smaller number (e.g., two or three) of recombinant kinases functionally expressed in vitro will be selected based on the results of the previous validation steps and their expected sensitivity profile to the tested inhibitors. Pilot HTS runs can be done with the kinase preparations in parallel with the corresponding commercially available analogs expressed in baculoviral system and the output data compared in terms of signal intensity, signal-to-background ratio and reproducibility.

Any or all of the processing steps described herein (e.g., translation, purification, quantitation, etc.) can be completed in a multiple parallel manner in multiwell (e.g., 96-well, 384-well or higher) format and can be easily automated.

Like the kinase assays described herein, the methods provided herein are used to produce a target protein in a functionally active form and provide a sufficiently sensitive screening assay platform. Functional cell-free expression of a wide variety of proteins in a functionally active form has been described in the literature. These include various non-tyrosine kinases, such as PKA (Foss et al. (1994) Eur J Biochem 220(1):217-23, EF-2K kinase (Redpath et al. (1996) J. Biol. Chem. 271(29):17547-17554), CHK2 (Xu et al. (2002) Mol. Cell Biol. 22:4419-4432), V-MOS (Herzog et al. (1990) J. Virol. 64(6):3093-3096), a number of plant kinases (Sawasaki et al. (2004) Phytochemistry 65:1549-1555), as well as other types of eukaryotic proteins (for reviews see Spirin, A. S. (Ed.) 2002, Cell-Free Translation Systems, Springer Verlag, Berlin-Heidelberg-New York; Swartz, J. A., 2003, Cell-Free Protein Expression, Springer Verlag, Berlin-Heidelberg-New York).

A variety of modern fluorescence or luminescence-based assay platforms are both highly sensitive and broadly adaptable for assaying various types of enzymatic activities or molecular interactions. One example is the commercial ALPHASCREEN™ (Perkin Elmer) platform mentioned herein. This is a chemiluminescent bead based, non-radioactive Amplified Luminescent Proximity Homogeneous Assay which allows detection down to the attomolar (10-18) level in some biological assays. The platform is highly versatile and, in addition to phosphotyrosine detection, is available in formats adapted for measuring binding of a variety of antibodies, affinity purification tags, and concentrations of cytokines, fluorescein, digoxin, cAMP, IP3, etc. Thus, the methods described herein can be adapted to develop highly sensitive assays for drug candidate screening for a majority of cellular drug targets.

5.3 Methods for Modulating Tyrosine Kinase Activity in a Patient

Provided herein are methods for modulating tyrosine kinase activity (e.g., the activity of one or more tyrosine kinases, such as PTK, RTK and/or CTK) in a patient, involving:
obtaining one or more polynucleotides that encode one or more polypeptides containing a tyrosine kinase domain, and/or fragment thereof,
optionally adding a first tag (e.g., a fluorescent tag) to the N-terminus of the one or more polynucleotides,
optionally adding a second tag (e.g., an affinity purification tag) to the C-terminus of the one or more polynucleotides,
translating the one or more polynucleotides in an in vitro cell-free translation system, wherein the resulting one or more polypeptides contain a tyrosine kinase domain, and/or fragment thereof, having kinase activity,
contacting a test compound with the one or more polypeptides,
detecting a modulation of kinase activity of the one or more polypeptides relative to kinase activity in the absence of test compound, and
administering the test compound to the patient, wherein the test compound modulates kinase activity in the patient relative to kinase activity in the absence of test compound.
   In specific embodiments, the one or more polynucleotides encode a panel of PTK (e.g., RTK and/or CTK), and/or fragments thereof (e.g., kinase domains and/or active fragments thereof), wherein substantially all of said PTKs in the panel have kinase activity. In certain embodiments, the one or more (such as about 5, 10, 15, 20, 30, 40, 50, 60, 70, 80, 90 or more, or such as about 100, 150, 200, 250, 300 350, 400, 450, 500 or more) polynucleotides encode for the same or different tyrosine kinases (e.g., PTK, RTK and/or CTK), and/or fragments thereof. In some embodiments, the one or more polynucleotides encode for tyrosine kinases, and/or fragments thereof, from different families and/or subfamilies of tyrosine kinases (see, e.g., FIG. 1, Manning et al. (2002) Science 298:1912)). In another embodiment, the one or more polynucleotides encode for tyrosine kinases, and/or fragments thereof, from the same family and/or subfamily of tyrosine kinases. In an embodiment, the one or more polynucleotides encode for tyrosine kinases, and/or fragments thereof, wherein the tyrosine kinases, and/or fragments thereof, are in different forms (e.g., wild-type and/or mutant forms) of the same kinase, different forms of different kinases, or combinations thereof. In a specific embodiment, the one or more polynucleotides encode a tyrosine kinase domain (or an active fragment thereof).

In certain embodiments, the panel of tyrosine kinases contains about 5, 10, 15, 20, 30, 40, 50, 60, 70, 80, 90 or more, or such as about 100, 150, 200, 250, 300 350, 400, 450, 500 or more tyrosine kinases, including the same or different families or subfamilies of tyrosine kinases, the same or different forms (e.g., wild-type or mutant) of tyrosine kinases, and/or fragments thereof, that are human non-receptor tyrosine kinases, human receptor tyrosine kinases, or a combination thereof (see Section 5.1 above). In certain embodiments, the panel of tyrosine kinases contains tyrosine kinases, and/or fragments thereof, selected from EGFR, IGF1R, KIT, VEGFR1, FGFR1, TRKA, MET, EPHB4, AXL, TIE1, DDR1, RET, ROS, ALK, ROR1, MUSK, SRC, ABL, JAK1, ACK1, FAK, FES, BRK, TEC, ZAP70, BLK, BMX, BTK, CSFR, CSK, CTK, DDR2, EPHA2, EPHA4, FGFR2, FGFR4, FGR, FLT3, FRK, FYN, HER2, HER3, JAK2, JAK3, KDR, LCK, LYN, PDGFRα, PYK2, SYK, TIE2, YES and combinations thereof.

In one embodiment, a polynucleotide encoding the tyrosine kinase is a linear polynucleotide. In some embodiments, kinase arrays (e.g., a panel of kinases and/or fragments thereof) are produced from PCR DNA in an IVT system. In certain embodiments, the one or more polynucleotides encoding the tyrosine kinases contain regulatory elements (see Sections 5.1.2 and 5.1.3 above). In some embodiments, the in vitro translation system is WGE, RRL and/or S30 cell-free translation system (see Section 5.1.5 above).

Non-limiting examples of test compounds that can be used in the methods provided herein include any protein, polypeptide, peptide, organic molecule, inorganic molecule, antibody, pharmaceutical, and/or candidate pharmaceutical that are natural products or prepared synthetically, and/or any compound found in the U.S. Pharmacopoeia (USP) and/or Physician's Desk Reference (59^{th} ed., 2005; 60^{th} ed., 2006), which are incorporated herein by reference in their entirety.

In certain embodiments, a test compound is screened against a panel (e.g., more than 5, more than 10, more than 25, more than 50, more than 100, more than 150, more than 200, more than 250, more than 300, more than 350, more than 400, more than 450, or more than 500) of different kinases and/or different forms of kinases, and/or active fragments thereof, simultaneously or in sequence. In other embodiments, more than one test compound (e.g., more than 5, more than 10, more than 25, more than 50, or more than 100) is screened against a panel (e.g., more than 5, more than 10, more than 25, more than 50, more than 100, more than 150, more than 200, more than 250, more than 300, more than 350, more than 400, more than 450, or more than 500) of different kinases and/or different forms of kinases, and/or active fragments thereof, simultaneously or in sequence. In some embodiments, the panel contains multiple naturally occurring kinase mutants, synthetically prepared kinase mutants, structurally comparable forms of one or more native (e.g., wild-type) kinases, and/or finctionally comparable forms of one or more native kinases. In certain embodiments, the screens are completed in a single reaction on a single test plate or a single reaction on multiple test plates. In other embodiments, the screens are performed in multiple reactions on a single test plate or multiple reactions on multiple test plates. In certain embodiments, the screening methods provided herein are high-throughput screens (HTS), e.g., in a 384-well or higher format (see Section 5.2 above).

Provided herein are methods for increasing tyrosine kinase activity (e.g., the activity of one or more tyrosine kinases, such as PTK, RTK and/or CTK) in a patient, involving:
providing one or more polynucleotides that encode one or more polypeptides containing a tyrosine kinase domain, and/or fragment thereof,
optionally adding a first tag (e.g., a fluorescent tag) to the N-terminus of the one or more polynucleotides,
optionally adding a second tag (e.g., an affinity purification tag) to the C-terminus of the one or more polynucleotides,
translating the one or more polynucleotides in an in vitro cell-free translation system, wherein the resulting one or more polypeptides contain a tyrosine kinase domain, and/or fragment thereof, having kinase activity,
contacting a test compound with the one or more polypeptides,
detecting an increase of kinase activity of the one or more polypeptides relative to kinase activity in the absence of test compound, and
administering the test compound to the patient, wherein the test compound increases kinase activity in the patient relative to kinase activity in the absence of test compound.

In certain embodiments, the panel of tyrosine kinases contains about 5, 10, 15, 20, 30, 40, 50, 60, 70, 80, 90 or more, or such as about 100, 150, 200, 250, 300 350, 400, 450, 500 or more tyrosine kinases, including the same or different families or subfamilies of tyrosine kinases, the same or different forms (e.g., wild-type or mutant) of tyrosine kinases, and/or fragments thereof, that are human non-receptor tyrosine kinases, human receptor tyrosine kinases, or a combination thereof. In certain embodiments, the panel of tyrosine kinases contains tyrosine kinases, and/or fragments thereof, selected from EGFR, IGF1R, KIT, VEGFR1, FGFR1, TRKA, MET, EPHB4, AXL, TIE1, DDR1, RET, ROS, ALK, ROR1, MUSK, SRC, ABL, JAK1, ACK1, FAK, FES, BRK, TEC, ZAP70, BLK, BMX, BTK, CSFR, CSK, CTK, DDR2, EPHA2, EPHA4, FGFR2, FGFR4, FGR, FLT3, FRK, FYN, HER2, HER3, JAK2, JAK3, KDR, LCK, LYN, PDGFRα, PYK2, SYK, TIE2, YES and combinations thereof.

In one embodiment, a polynucleotide encoding the tyrosine kinase is a linear polynucleotide. In some embodiments, kinase arrays (e.g., a panel of kinases and/or fragments thereof) are produced from PCR DNA in an IVT system. In certain embodiments, the one or more polynucleotides encoding the tyrosine kinases contain regulatory elements (see Sections 5.12 and 5.13 above). In some embodiments, the in vitro translation system is WGE, RRL and/or S30 cell-free translation system (see Section 5.1.5 above).

Non-limiting examples of test compounds that can be used in the methods provided herein for increasing tyrosine kinase activity in a patient include any protein, polypeptide, peptide, organic molecule, inorganic molecule, antibody, pharmaceutical, and/or candidate pharmaceutical that are natural products or prepared synthetically, and/or any compound found in the U.S. Pharmacopoeia (USP) and/or Physician's Desk Reference (59^{th} ed., 2005; 60^{th} ed., 2006), which are incorporated herein by reference in their entirety.

In certain embodiments, a test compound is screened against a panel (e.g., more than 5, more than 10, more than 25, more than 50, more than 100, more than 150, more than 200, more than 250, more than 300, more than 350, more than 400, more than 450, or more than 500) of different kinases and/or different forms of kinases, and/or active fragments thereof, simultaneously or in sequence. In other embodiments, more than one test compound (e.g., more than 5, more than 10, more than 25, more than 50, or more than 100) is screened against a panel (e.g., more than 5, more than 10, more than 25, more than 50, more than 100, more than 150, more than 200, more than 250, more than 300, more than 350, more than 400, more than 450, or more than 500) of different kinases and/or different forms of kinases, and/or active fragments thereof, simultaneously or in sequence. In some embodiments, the panel contains multiple naturally occurring kinase mutants, synthetically prepared kinase mutants, structurally comparable forms of one or more native (e.g., wild-type) kinases, and/or functionally comparable forms of one or more native kinases. In certain embodiments, the screens are completed in a single reaction on a single test plate or a single reaction on multiple test plates. In other embodiments, the screens are performed in multiple reactions on a single test plate or multiple reactions on multiple test plates. In certain embodiments, the screening methods provided herein are high-throughput screens (HTS), e.g., in a 384-well or higher format (see Section 5.2 above).

Also provided herein are methods for decreasing tyrosine kinase activity (e.g., the activity of one or more tyrosine kinases, such as PTK, RTK and/or CTK) in a patient, involving:
obtaining one or more polynucleotides that encode one or more polypeptides containing a tyrosine kinase domain, and/or fragment thereof,
optionally adding a first tag (e.g., a fluorescent tag) to the N-terminus of the one or more polynucleotides,
optionally adding a second tag (e.g., an affinity purification tag) to the C-terminus of the one or more polynucleotides,
translating the one or more polynucleotides in an in vitro cell-free translation system, wherein the resulting one or more polypeptides contain a tyrosine kinase domain, and/or fragment thereof, having kinase activity,
contacting a test compound with the one or more polypeptides,
detecting a decrease of kinase activity of the one or more polypeptides relative to kinase activity in the absence of test compound, and
administering the test compound to the patient, wherein the test compound decreases kinase activity in the patient relative to kinase activity in the absence of test compound.

In certain embodiments, the panel of tyrosine kinases contains about 5, 10, 15, 20, 30, 40, 50, 60, 70, 80, 90 or more, or such as about 100, 150, 200, 250, 300 350, 400, 450, 500 or more tyrosine kinases, including the same or different families or subfamilies of tyrosine kinases, the same or different forms (e.g., wild-type or mutant) of tyrosine kinases, and/or fragments thereof, that are human non-receptor tyrosine kinases, human receptor tyrosine kinases, or a combination thereof. In certain embodiments, the panel of tyrosine kinases contains tyrosine kinases, and/or fragments thereof, selected from EGFR, IGF1R, KIT, VEGFR1, FGFR1, TRKA, MET, EPHB4, AXL, TIE1, DDR1, RET, ROS, ALK, ROR1, MUSK, SRC, ABL, JAK1, ACK1, FAK, FES, BRK, TEC, ZAP70, BLK, BMX, BTK, CSFR, CSK, CTK, DDR2, EPHA2, EPHA4, FGFR2, FGFR4, FGR, FLT3, FRK, FYN, HER2, HER3, JAK2, JAK3, KDR, LCK, LYN, PDGFRα, PYK2, SYK, TIE2, YES and combinations thereof.

In one embodiment, a polynucleotide encoding the tyrosine kinase is a linear polynucleotide. In some embodiments, kinase arrays (e.g., a panel of kinases and/or fragments thereof) are produced from PCR DNA in an IVT system. In certain embodiments, the one or more polynucleotides encoding the tyrosine kinases contain regulatory elements (see Sections 5.12 and 5.13 above). In some embodiments, the in vitro translation system is WGE, RRL and/or S30 cell-free translation system (see Section 5.1.5 above).

Non-limiting examples of test compounds that can be used in the methods provided herein for decreasing tyrosine kinase activity in a patient include any protein, polypeptide, peptide, organic molecule, inorganic molecule, antibody, pharmaceutical, and/or candidate pharmaceutical that are natural products or prepared synthetically, and/or any compound found in the U.S. Pharmacopoeia (USP) and/or Physician's Desk Reference (59^{th} ed., 2005; 60^{th} ed., 2006), which are incorporated herein by reference in its entirety.

In certain embodiments, a test compound is screened against a panel (e.g., more than 5, more than 10, more than 25, more than 50, more than 100, more than 150, more than 200, more than 250, more than 300, more than 350, more than 400, more than 450, or more than 500) of different kinases and/or different forms of kinases, and/or active fragments thereof, simultaneously or in sequence. In other embodiments, more than one test compound (e.g., more than 5, more than 10, more than 25, more than 50, or more than 100) is screened against a panel (e.g., more than 5, more than 10, more than 25, more than 50, more than 100, more than 150, more than 200, more than 250, more than 300, more than 350, more than 400, more than 450, or more than 500) of different kinases and/or different forms of kinases, and/or active fragments thereof, simultaneously or in sequence. In some embodiments, the panel contains multiple naturally occurring kinase mutants, synthetically prepared kinase mutants, structurally comparable forms of one or more native (e.g., wild-type) kinases, and/or functionally comparable forms of one or more native kinases. In certain embodiments, the screens are completed in a single reaction on a single test plate or a single reaction on multiple test plates. In other embodiments, the screens are performed in multiple reactions on a single test plate or multiple reactions on multiple test plates. In certain embodiments, the screening methods provided herein are high-throughput screens (HTS), e.g., in a 384-well or higher format (see Section 5.2 above).

In any of the methods provided herein, the test compound can be administered to the patient in the form of a pharmaceutical composition.

5.3.1 Pharmaceutical Compositions

Therapeutic formulations containing a PTK agonist, antagonist or other modulator identified by the methods provided herein can be prepared for storage by mixing the agonist, antagonist or other modulator having the desired degree of purity with optional physiologically acceptable carriers, excipients or stabilizers (Remington's Pharmaceutical Sciences (1990) Mack Publishing Co., Easton, Pa., which is hereby incorporated by reference in its entirety), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The PTK agonists, antagonists, or other modulators identified by the methods provided herein can also be formulated in liposomes. Liposomes containing the molecule of interest are prepared by methods known in the art, such as described in Epstein et al. (1985) Proc. Natl. Acad. Sci. USA 82:3688; Hwang et al. (1980) Proc. Natl. Acad. Sci. USA 77:4030; and U.S. Pat. Nos. 4,485,045 and 4,544,545. Liposomes with enhanced circulation time are disclosed in U.S. Pat. No. 5,013,556.

Particularly useful immunoliposomes can be generated by the reverse phase evaporation method with a lipid composition containing phosphatidylcholine, cholesterol and PEG-derivatized phosphatidylethanolamine (PEG-PE). Liposomes are extruded through filters of defined pore size to yield liposomes with the desired diameter. Fab′ fragments of an antibody provided herein can be conjugated to the liposomes as described in Martin et al. (1982) J. Biol. Chem. 257:286-288 via a disulfide interchange reaction. A chemotherapeutic agent (such as Doxorubicin) is optionally contained within the liposome; See Gabizon et al., (1989) J. National Cancer Inst. 81(19):1484.

The formulation herein can also contain more than one active compound as necessary for the particular indication being treated. In certain embodiments, those with complementary activities that do not adversely affect each other. Such molecules are suitably present in combination in amounts that are effective for the purpose intended. For example, a PTK antagonist, antagonist or other modulator identified by the methods provided herein can be combined with one or more other therapeutic agents. Such combined therapy can be administered to the patient serially or simultaneously or in sequence.

The active ingredients can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (1990) Mack Publishing Co., Easton, Pa., which is hereby incorporated by reference in its entirety.

The formulations to be used for in vivo administration can be sterile. This is readily accomplished by filtration through, e.g., sterile filtration membranes.

Sustained-release preparations can also be prepared. Suitable examples of sustained-release preparations include semipermeable matrices of solid hydrophobic polymers containing the antagonist, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPO™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The pharmaceutical compositions provided herein contain therapeutically effective amounts of one or more of the modulators (e.g., agonists, antagonists, inhibitors) of kinase activity provided herein that are useful in the prevention, treatment, or amelioration of one or more of the symptoms of diseases or disorders associated with a kinase (e.g., a PTK, RTK or CTK), or in which a kinase is implicated, in a pharmaceutically acceptable carrier.

For example, aberrant cell signaling by protein kinases (e.g., a PTK, RTK or CTK) can lead to many diseases or disorder. Non-limiting examples of such diseases or disorders include a variety of cancers (or tumors or other types of aberrant cell growths), type II diabetes, Alzheimer's disease, and other autoimmune, inflammatory, metabolic and neurodegenerative diseases. Depending on the particular kinase-disease association, both kinase agonists and antagonists (inhibitors) can have therapeutic values. For example, agonists of the insulin receptor, a tyrosine kinase, can be used to treat diabetes. Many of the approximately 90 members of the human tyrosine kinase family are implicated in cancer due to their crucial role in controlling such biological processes as angiogenesis, cell motility and invasion; and cell proliferation and apoptosis. A large number of tyrosine kinase inhibitors are currently in clinical or preclinical development for oncology indications. As yet another example, modulators of the MAP kinase pathway, which is critical for cellular responses to various external stimuli, can be used for treatment of diseases spanning diverse therapeutic areas such as rheumatoid arthritis, Crohn's disease, ischemic stroke, Parkinson's disease, and prostate cancer. Additional diseases and disorders amenable to prevention or treatment are discussed in greater detail in Section 5.3.2 below.

Pharmaceutical carriers suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients.

The compositions contain one or more compounds identified using the methods provided herein. The compounds are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel (1985) Introduction to Pharmaceutical Dosage Forms, 4^{th} Ed., p. 126).

In the compositions, effective concentrations of one or more compounds or pharmaceutically acceptable derivatives thereof is (are) mixed with a suitable pharmaceutical carrier. The compounds may be derivatized as the corresponding salts, esters, enol ethers or esters, acetals, ketals, orthoesters, hemiacetals, hemiketals, solvates, hydrates or prodrugs prior to formulation, as described above. The concentrations of the compounds in the compositions are effective for delivery of an amount, upon administration, that treats, prevents, or ameliorates one or more of the symptoms of diseases or disorders associated with kinase activity (e.g., a PTK, RTK or CTK) or in which kinase activity is implicated.

In one embodiment, the compositions are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

The active compound is included in the pharmaceutically acceptable carrier in an amount sufficient to exert a therapeutically useful effect in the absence of undesirable side effects on the patient treated. The therapeutically effective concentration can be determined empirically by testing the compounds in in vitro and in vivo systems using routine methods and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders associated with kinase (e.g., a PTK, RTK or CTK) activity or in which kinase activity is implicated, as described herein.

In one embodiment, a therapeutically effective dosage produces a serum concentration of active ingredient of from about 0.1 ng/ml to about 50-100 μg/ml. The pharmaceutical compositions, in another embodiment, provide a dosage of from about 0.001 mg to about 2000 mg of compound per kilogram of body weight per day. Pharmaceutical dosage unit forms can be prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The active ingredient can be administered at once, or may be divided into a number of smaller doses to be administered at intervals of time. It is understood that the precise dosage and duration of treatment is a function of the disease being treated and can be determined empirically using known testing protocols or by extrapolation from in vivo or in vitro test data. It is to be noted that concentrations and dosage values can also vary with the severity of the condition to be alleviated. It is to be further understood that for any particular subject, specific dosage regimens can be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds can be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds can also be used in formulating effective pharmaceutical compositions.

Upon mixing or addition of the compound(s), the resulting mixture can be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered can also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences (1990) Mack Publishing Co., Easton, Pa., which is hereby incorporated by reference in its entirety.

Dosage forms or compositions containing active ingredient in the range of 0.005% to 100% with the balance made up from non-toxic carrier can be prepared. Methods for preparation of these compositions are known to those skilled in the art. The contemplated compositions can contain 0.001%-100% active ingredient, in one embodiment 0.1-95%, in another embodiment 75-85%.

5.3.1.1 Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules can be hard or soft gelatin capsules, while granules and powders can be provided in non-effervescent or effervescent form with the combination of other ingredients known to those skilled in the art.

In certain embodiments, the formulations are solid dosage forms. In certain embodiments, the formulations are capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, can be provided in a composition that protects it from the acidic environment of the stomach. For example, the composition can be formulated in an enteric coating that maintains its integrity in the stomach and releases the active compound in the intestine. The composition can also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations can be coated as known by those of skill in the art in order to modify or sustain dissolution of the active ingredient. Thus, for example, they may be coated with a conventional enterically digestible coating, such as phenylsalicylate, waxes and cellulose acetate phthalate.

In some embodiments, the formulations are liquid dosage forms. Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is, in one embodiment, encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, can be diluted with a sufficient quantity of a pharmaceutically acceptable liquid carrier, e.g., water, to be easily measured for administration.

Alternatively, liquid or semi-solid oral formulations can be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

5.3.1.2 Injectables, Solutions and Emulsions

Parenteral administration, in one embodiment characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations can be packaged in an ampoule, a vial or a syringe with a needle. All preparations for parenteral administration can be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound can be suspended in micronized or other suitable form or can be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

5.3.1.3 Lyophilized Powders

In other embodiments, the pharmaceutical formulations are lyophilized powders, which can be reconstituted for administration as solutions, emulsions and other mixtures. They may also be reconstituted and formulated as solids or gels.

The lyophilized powder is prepared by dissolving a compound provided herein, or a pharmaceutically acceptable derivative thereof, in a suitable solvent. In some embodiments, the lyophilized powder is sterile. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

5.3.1.4 Topical Administration

Topical mixtures are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof can be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

The compounds can be formulated for local or topical application, such as for topical application to the skin and mucous membranes, such as in the eye, in the form of gels, creams, and lotions and for application to the eye or for intracistemal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5.3.1.5 Compositions for Other Routes of Administration

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010,715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm.

Tablets and capsules for rectal administration can be manufactured using the same pharmaceutically acceptable substance and by the same methods as for formulations for oral administration.

5.3.1.6 Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These can be prepared according to methods known to those skilled in the art. For example, liposome formulations can be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

5.3.2 Therapy with Compounds Provided Herein

The methods provided herein encompass a method of preventing or treating a protein kinase-associated disorder or disease state, the method involving administering a therapeutically effective amount of a test compound identified using the IVT system and methods provided herein. As used herein the term “protein kinase-associated disease state” refers to those disorders which result from aberrant protein kinase activity and/or which are alleviated by inhibition (or, in some cases, by activation) of one or more of these enzymes.

In one embodiment, the disease state involves a RTK selected from EGF, HER2, HER3, HER4, IR, IGF-1R, IRR, PDGFRα, PDGFRβ, CSFIR, C-KIT, C-FMS, FLK-1R, FLK4, KDR/FLK-1, FLT-1, FGFR-1R, FGFR-2R, FGFR-3R AND FGFR-4R.

In another embodiment, the disease state involves a CTK selected from SRC, FRK, BTK, CSK, ABL, ZAP70, FES/FPS, FAK, ACK, YES, FYN, LYN, LCK, BLK, HCK, FGR and YRK.

In a yet another embodiment, the disease state involves a tyrosine kinase selected from JAK1, JAK2, JAK3 and TYK2.

In a some embodiments, the disease state is selected from atopy, such as allergic asthma, atopic dermatitis (eczema), and allergic rhinitis; cell mediated hypersensitivity, such as allergic contact dermatitis and hypersensitivity pneumonitis; rheumatic diseases, such as systemic lupus erythematosus (SLE), rheumatoid arthritis, juvenile arthritis, Sjogren's syndrome, scleroderma, polymyositis, ankylosing spondylitis, psoriatic arthritis; other autoimmune diseases such as type I diabetes, autoimmune thyroid disorders, and Alzheimer's disease; viral diseases, such as Epstein Barr virus (EBV), hepatitis B, hepatitis C, HIV, HTLV 1, Varicella-Zoster virus (VZV), Human Papilloma virus (HPV), cancer, such as leukemia, lymphoma and prostate cancer.

5.3.2.1 Therapy with PTK Antagonists

For therapeutic applications, the antagonists identified by the methods provided herein are administered to a mammal, such as a human, in a pharmaceutically acceptable dosage form such as those discussed above, including those that may be administered to a human intravenously as a bolus or by continuous infusion over a period of time, by intramuscular, intraperitoneal, intracerebrospinal, subcutaneous, intraarticular, intrasynovial, intrathecal, oral, topical, or inhalation routes. The antagonists also are suitably administered by intratumoral, peritumoral, intralesional, or perilesional routes, to exert local as well as systemic therapeutic effects. The intraperitoneal route is expected to be particularly useful, for example, in the treatment of ovarian tumors.

For the prevention or treatment of disease, the appropriate dosage of antagonist will depend on the type of disease to be treated, as defined above, the severity and course of the disease, whether the antagonist is administered for preventive or therapeutic purposes, previous therapy, the patient's clinical history and response to the antagonist, and the discretion of the attending physician. The antagonist is suitably administered to the patient at one time or over a series of treatments.

The antagonists are useful in the treatment of various neoplastic and non-neoplastic diseases and disorders. Cancers and related conditions that are amenable to treatment include breast carcinomas, lung carcinomas, gastric carcinomas, esophageal carcinomas, colorectal carcinomas, liver carcinomas, ovarian carcinomas, thecomas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema (such as that associated with brain tumors), and Meigs' syndrome.

Non-neoplastic conditions that are amenable to treatment include rheumatoid arthritis, psoriasis, atherosclerosis, diabetic and other proliferative retinopathies including retinopathy of prematurity, retrolental fibroplasia, neovascular glaucoma, age-related macular degeneration, thyroid hyperplasias (including Grave's disease), corneal and other tissue transplantation, chronic inflammation, lung inflammation, nephrotic syndrome, preeclampsia, ascites, pericardial effusion (such as that associated with pericarditis), and pleural effusion.

Depending on the type and severity of the disease, about 1 μg/kg to about 100 mg/kg (e.g., 0.1-20 mg/kg) of antagonist is an initial candidate dosage for administration to the patient, whether, for example, by one or more separate administrations, or by continuous infusion. A typical daily or weekly dosage might range from about 1 μg/kg to about 20 mg/kg or more, depending on the factors mentioned above. For repeated administrations over several days or longer, depending on the condition, the treatment is repeated until a desired suppression of disease symptoms occurs. However, other dosage regimens may be useful. The progress of this therapy is easily monitored by conventional techniques and assays, including, for example, radiographic tumor imaging.

According to another embodiment, the effectiveness of the antagonist in preventing or treating disease may be improved by administering the antagonist simultaneously or serially or in combination with another therapy that is effective for those purposes, such as tumor necrosis factor (TNF), an antagonist capable of inhibiting or neutralizing the angiogenic activity of acidic or basic fibroblast growth factor (FGF) or hepatocyte growth factor (HGF), an antagonist capable of inhibiting or neutralizing the coagulant activities of tissue factor, protein C, or protein S (see Esmon et a., PCT Patent Publication No. WO 91/01753, published 21 Feb. 1991), an antagonist such as an antibody capable of binding to HER2 receptor (see U.S. Pat. No. 5,772,997), or one or more conventional therapeutic agents such as, for example, alkylating agents, folic acid antagonists, anti-metabolites of nucleic acid metabolism, antibiotics, pyrimidine analogs, 5-fluorouracil, cisplatin, purine nucleosides, amines, amino acids, triazol nucleosides, or corticosteroids. Such other therapeutic agents may be present in the composition being administered or may be administered separately. Also, the antagonist is suitably administered serially or in combination with radiological treatments, whether involving irradiation or administration of radioactive substances.

In one embodiment, vascularization of tumors is attacked in combination therapy. The antagonist and one or more other antagonists are administered to tumor-bearing patients at therapeutically effective doses as determined for example by observing necrosis of the tumor or its metastatic foci, if any. This therapy is continued until such time as no further beneficial effect is observed or clinical examination shows no trace of the tumor or any metastatic foci. Then TNF is administered, alone or in combination with an auxiliary agent such as α-, β-, or γ-interferon, anti-HER2 antibody, heregulin, anti-heregulin antibody, D-factor, interleukin-1 (IL-1), interleukin-2 (IL-2), granulocyte-macrophage colony stimulating factor (GM-CSF), or agents that promote microvascular coagulation in tumors, such as anti-protein C antibody, anti-protein S antibody, or C4b binding protein (see Esmon et al., PCT Patent Publication No. WO 91/01753, published 21 Feb. 1991), or heat or radiation.

Since the auxiliary agents will vary in their effectiveness, it is desirable to compare their impact on the tumor by matrix screening in conventional fashion. The administration of antagonist and TNF is repeated until the desired clinical effect is achieved. Alternatively, the antagonist is administered together with TNF and, optionally, auxiliary agent(s). In instances where solid tumors are found in the limbs or in other locations susceptible to isolation from the general circulation, the therapeutic agents described herein are administered to the isolated tumor or organ. In other embodiments, a FGF or platelet-derived growth factor (PDGF) antagonist, such as an anti-FGF or an anti-PDGF neutralizing antibody, is administered to the patient in conjunction with the antagonist. Treatment with antagonist optimally may be suspended during periods of wound healing or desirable vascularization.

In one embodiment, the PTK antagonist is used to treat a vascular anomaly (e.g., small vessel anomalies) resulting from estrogen therapy. Administration of the PTK antagonist may precede or follow administration of estrogen to the patient. Concomitant therapy is also contemplated, for example, where the RTK antagonist and estrogen are provided in the same composition. The dosages of estrogen may correspond to those previously known.

5.3.2.2 Therapy with PTK Agonists

Also provided herein is a method of stimulating angiogenesis involving administering an PTK agonist identified by the IVT methods provided herein to a mammal. For example, the agonist may be used to treat conditions associated with the vascular endothelium, such as the treatment of trauma to the vascular network. Examples of such trauma that could be so treated include, but are not limited to, surgical incisions, particularly those involving the heart, wounds, including lacerations, incisions, and penetrations of blood vessels, and surface ulcers involving the vascular endothelium such as diabetic, haemophiliac, and varicose ulcers.

For the traumatic indications referred to above, the PTK agonist will be formulated and dosed in a fashion consistent with good medical practice taking into account the specific disorder to be treated, the condition of the individual patient, the site of delivery of the RTK agonist, the method of administration, and other factors known to practitioners.

Additional indications for the PTK agonist are in the treatment of full-thickness wounds such as dermal ulcers, including the categories of pressure sores, venous ulcers, and diabetic ulcers, as well as of full-thickness burns and injuries where angiogenesis is required to prepare the bum or injured site for a skin graft or flap. In this case the PTK agonist is either applied directly to the site or it is used to soak the skin or flap that is being transplanted prior to grafting. In a similar fashion, the PTK agonist can be used in plastic surgery when reconstruction is required following a burn or other trauma, or for cosmetic purposes.

Angiogenesis is also important in keeping wounds clean and non-infected. The PTK agonist can therefore be used in association with general surgery and following the repair of cuts and lacerations. It is particularly useful in the treatment of abdominal wounds with a high risk of infection. Vascularization is also key to fracture repair, since blood vessels develop at the site of bone injury. Administration of the PTK agonist to the site of a fracture is therefore another utility.

In cases where the PTK agonist is being used for topical wound healing, as described above, it may be administered by any of the routes described herein for the re-endothelialization of vascular tissue. In certain embodiments, the PTK agonist is administered by topical means. In these cases, it will be administered as either a solution, spray, gel, cream, ointment, or dry powder directly to the site of injury. Slow-release devices directing the PTK agonist to the injured site will also be used. In topical applications, the PTK agonist will be applied at a concentration ranging from about 50 to 1,000 μg/mL, either in a single application, or in dosing regimens that are daily or every few days for a period of one week to several weeks. Generally, the amount of topical formulation administered is that which is sufficient to apply from about 0.1 to 100 μg/cm² of the PTK agonist, based on the surface area of the wound.

The PTK agonist can be used as a post-operative wound healing agent in balloon angioplasty, a procedure in which vascular endothelial cells are removed or damaged, together with compression of atherosclerotic plaques. The PTK agonist can be applied to inner vascular surfaces by systemic or local intravenous application either as intravenous bolus injection or infusions. If desired, the PTK agonist can be administered over time using a micrometering pump. Suitable compositions for intravenous administration contain the PTK agonist in an amount effective to promote endothelial cell growth and a parenteral carrier material. The PTK agonist can be present in the composition over a wide range of concentrations, for example, from about 50 μg/mL to about 1,000 μg/mL using injections of 3 to 10 mL per patient, administered once or in dosing regimens that allow for multiple applications. Any of the known parenteral carrier vehicles can be used, such as normal saline or 5-10% dextrose.

The PTK agonist can also be used to promote endothelialization in vascular graft surgery. In the case of vascular grafts using either transplanted vessels or synthetic material, for example, the PTK agonist can be applied to the surfaces of the graft and/or at the junctions of the graft and the existing vasculature to promote the growth of vascular endothelial cells. For such applications, the PTK agonist can be applied intravenously as described above for balloon angioplasty or it can be applied directly to the surfaces of the graft and/or the existing vasculature either before or during surgery. In such cases it may be desired to apply the PTK agonist in a thickened carrier material so that it will adhere to the affected surface. Suitable carrier materials include, for example, 1-5% carbopol. The PTK agonist can be present in the carrier over a wide range of concentrations, for example, from about 50 μg/mg to about 1,000 μg/mg. Alternatively, the PTK agonist can be delivered to the site by a micrometering pump as a parenteral solution.

The PTK agonist can also be employed to repair vascular damage following myocardial infarction and to circumvent the need for coronary bypass surgery by stimulating the growth of a collateral circulation. The PTK agonist is administered intravenously for this purpose, either in individual injections or by micrometering pump over a period of time as described above or by direct infusion or injection to the site of damaged cardiac muscle.

The route of PTK agonist administration is in accord with known methods, e.g., those routes set forth above for specific indications, as well as the general routes of injection or infusion by intravenous, intraperitoneal, intracerebral, intramuscular, intraocular, intraarterial, or intralesional means, or sustained release systems as noted below. PTK agonist is administered continuously by infusion or by bolus injection. Generally, where the disorder permits, one should formulate and dose the PTK agonist for site-specific delivery. This is convenient in the case of wounds and ulcers.

An effective amount of PTK agonist to be employed therapeutically will depend, for example, upon the therapeutic objectives, the route of administration, and the condition of the patient. Accordingly, it will be necessary for the therapist to titer the dosage and modify the route of administration as required to obtain the optimal therapeutic effect. Typically, the clinician will administer the PTK agonist until a dosage is reached that achieves the desired effect. A typical daily dosage for systemic treatment might range from about 1 μg/kg to up to 10 mg/kg or more, depending on the factors mentioned above. As an alternative general proposition, the PTK agonist is formulated and delivered to the target site or tissue at a dosage capable of establishing in the tissue a PTK agonist level greater than about 0.1 ng/cc up to a maximum dose that is efficacious but not unduly toxic. This intra-tissue concentration should be maintained if possible by continuous infusion, sustained release, topical application, or injection at empirically determined frequencies. The progress of this therapy is easily monitored by conventional assays.

It is within the scope hereof to combine the PTK agonist therapy with other novel or conventional therapies (e.g., growth factors such as VEGF, acidic or basic fibroblast growth factor (aFGF or bFGF, respectively), platelet-derived growth factor (PDGF), insulin-like growth factor (IGF-I or IGF-II), nerve growth factor (NGF), anabolic steroids, EGF or TGF-β) for enhancing the activity of any of the growth factors, including the PTK agonist, in promoting cell proliferation and repair. It is not necessary that such co-treatment drugs be included per se in the compositions provided herein, although this will be convenient where such drugs are proteinaceous. Such admixtures are suitably administered in the same manner and for the same purposes as the PTK agonist used alone. The useful molar ratio of PTK agonist to such secondary growth factors is typically 1:0.1-10. In some embodiments, equimolar amounts are used.

5.4 Kits

Provided herein are kits for screening for a modulator (an agonist, antagonist and/or any other type of activator or inhibitor) of tyrosine kinase activity (e.g., the activity of one or more tyrosine kinases in a panel of tyrosine kinases) containing:
one or more polynucleotides that encode for one or more polypeptides containing a tyrosine kinase domain, and/or fragment thereof, wherein said polynucleotides optionally further contains a first tag (e.g., a fluorescent tag) on the N-terminus of the one or more polynucleotide and/or a second tag (e.g., an affinity purification tag) on the C-terminus of the one or more polynucleotides, and
an in vitro translation system.

In certain embodiments, the panel of tyrosine kinases contains about 5, 10, 15, 20, 30, 40, 50, 60, 70, 80, 90 or more, or such as about 100, 150, 200, 250, 300 350, 400, 450, 500 or more tyrosine kinases, including the same or different families or subfamilies of tyrosine kinases, the same or different forms (e.g., wild-type or mutant) of tyrosine kinases, and/or fragments thereof, that are human non-receptor tyrosine kinases, human receptor tyrosine kinases, or a combination thereof. In certain embodiments, the panel of tyrosine kinases contains tyrosine kinases, and/or fragments thereof, selected from EGFR, IGF1R, KIT, VEGFR1, FGFR1, TRKA, MET, EPHB4, AXL, TIE1, DDR1, RET, ROS, ALK, ROR1, MUSK, SRC, ABL, JAK1, ACK1, FAK, FES, BRK, TEC, ZAP70, BLK, BMX, BTK, CSFR, CSK, CTK, DDR2, EPHA2, EPHA4, FGFR2, FGFR4, FGR, FLT3, FRK, FYN, HER2, HER3, JAK2, JAK3, KDR, LCK, LYN, PDGFRα, PYK2, SYK, TIE2, YES and combinations thereof. In some embodiments, the in vitro cell-free translation system is WGE, RRL, S30, or a combination thereof. In one embodiment, a polynucleotide encoding the tyrosine kinase is a linear polynucleotide.

5.7 Articles of Manufacture

The compounds or pharmaceutically acceptable derivatives may be packaged as articles of manufacture containing packaging material, a compound or pharmaceutically acceptable derivative thereof provided herein, which is effective for modulating the activity of a kinase (e.g., a PTK, RTK or CTK), or for treatment, prevention or amelioration of one or more symptoms of kinase-mediated diseases or disorders, or diseases or disorders in which kinase activity, is implicated, within the packaging material, and a label that indicates that the compound or composition, or pharmaceutically acceptable derivative thereof, is used for modulating kinase activity, or for treatment, prevention or amelioration of one or more symptoms of a kinase-mediated disease or disorder, or diseases or disorders in which kinase activity is implicated.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder in which kinase activity is implicated as a mediator or contributor to the symptoms or cause.

The following examples are provided for illustrative purposes only and are not intended to limit the scope of the IVT systems and methods provided herein.

### 6. EXAMPLES

6.1 Expression of IGF1R Tyrosine Kinase

6.1.1 Construction of a Kinase Expression Cassette

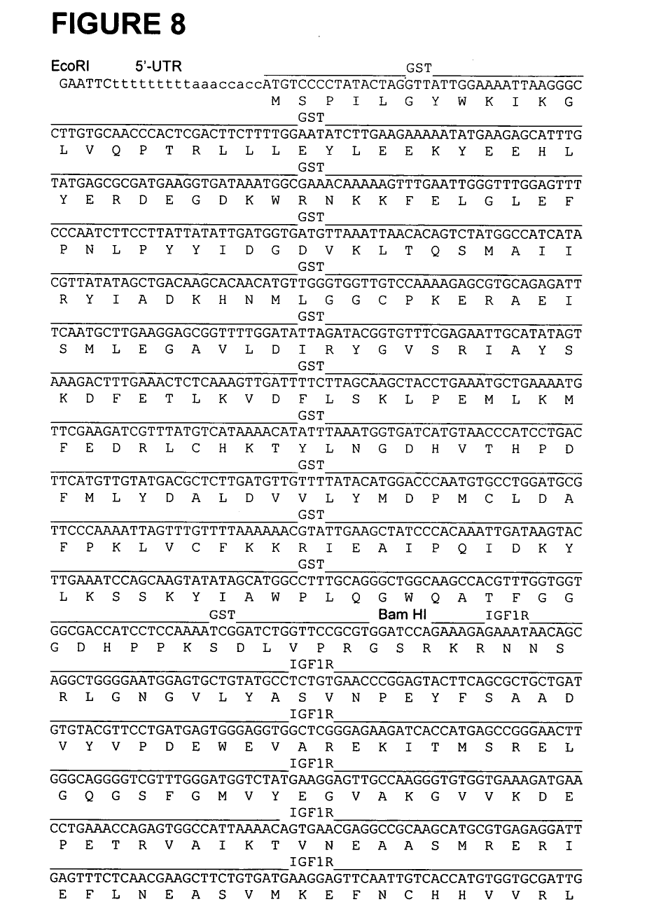

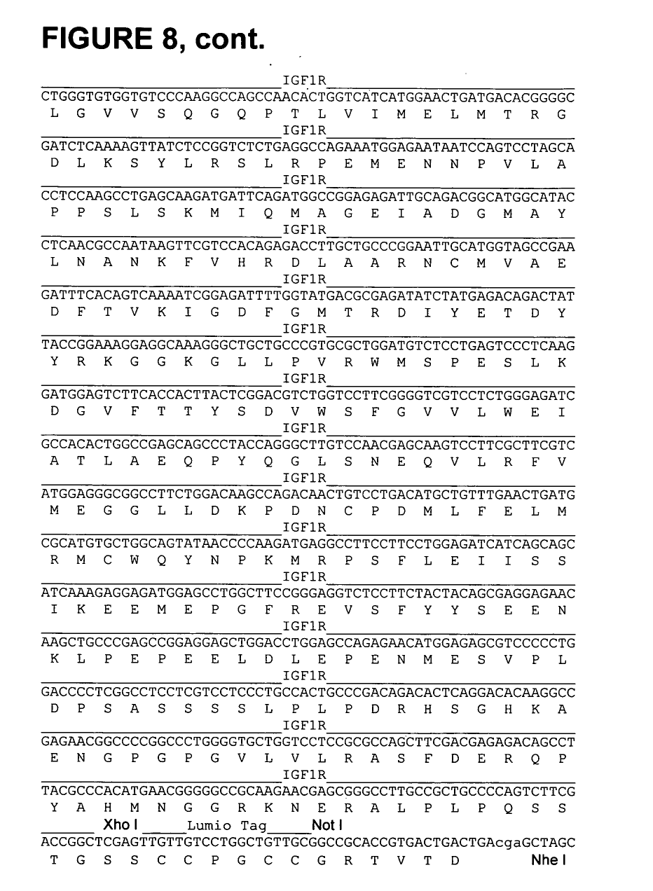

Structure of an expression cassette encoding glutathione S-transferase (GST)-IGF1R fusion protein is shown in the FIG. 8. The cassette also contains an idealized synthetic 5′-UTR (Zozulya et al. (1990). Protein Eng 3(5):453-8), consensus Kozak sequence, and LUMIO™ tag sequence at the C-terminus of the fusion protein. Design of the expression cassette allows simple swapping of the kinase domain modules such as those shown in FIG. 5 and FIG. 6, by subcloning into the Bam HI-XhoI restriction sites of the cassette.

6.1.2 Construction of an In Vitro Transcription-translation Vector

The complete cassette can be introduced into a given transcription vector via EcoRI and NheI (XbaI) sites to place the translation module under control of the desired promoter, such as bacteriophage promoters T7, SP6 or T3. For the experiments described below, the GST-IGF1R expression cassette (FIG. 8) was subcloned in the pGEM1 or PGEM4Z vector (Promega Corp., Madsion, Wis.) to allow T7- or SP6-driven transcription in vitro, respectively.

6.1.3 Coupled Transcription-translation in RRL

TNT® T7 Quick Coupled Transcription/Translation System (Promega Corp., Cat. L1170) or TNT® SP6 Quick Coupled Transcription/Translation System (Promega Corp., Cat. L2080) was used for expression of the GST-IGF1R expression cassette from a circular plasmid template according to the manufacturer's experimental protocol.

In a typical reaction, 40 μl of TNT® T7 Quick Master Mix was supplied with 40 μM methionine and 2 μg of the corresponding plasmid template DNA in a total volume of 50 μl and incubated for 1 hr at 30° C. Immediately after translation, dilution of the crude translation mix were used in ALPHASCREEN™ Phosphotyrosine Assay (PerkinElmer, Cat. PT66) to detect kinase activity of the GST-IGF1R fusion protein. Alternatively, the fusion protein was affinity purified prior to the assay, so as to eliminate optically absorbing components of the lysate, such as hemoglobin that may obscure chemiluminescent signal detection in the kinase assay. To purify the protein, lysates were transferred into the wells of REACTI-BIND™ glutathione-coated plate (Pierce, Cat. 15140), incubated for 15 min at 4° C. with occasional shaking, and then washed once with kinase assay buffer containing 0.1% BSA (see below) and eluted with 25 μl of 10 mM reduced glutathione in the kinase buffer for 15 min at 4° C. Alternatively, GST-fused kinases were purified using MagneGST™ glutathione particles (Promega Corp.; Cat. V8611). Serial dilutions of the eluted sample were immediately analyzed in ALPHASCREEN™ assay.

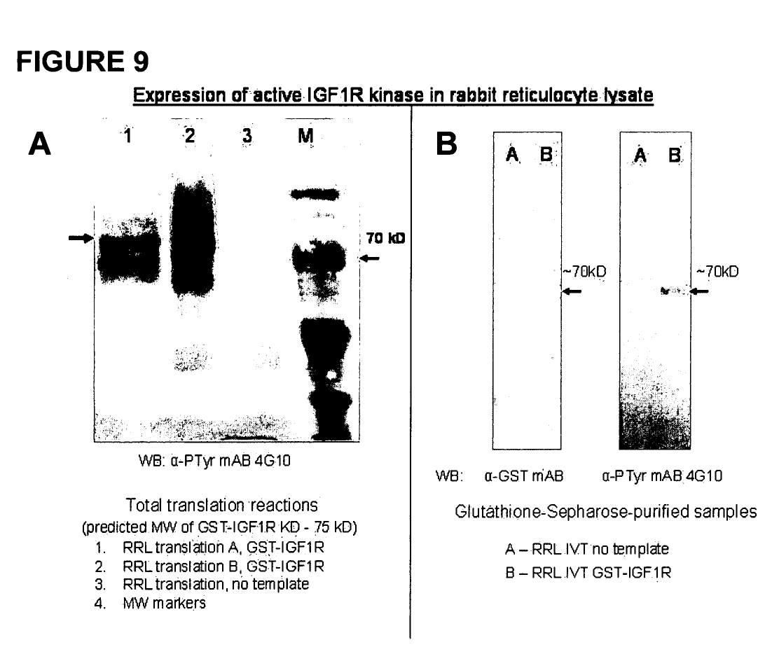

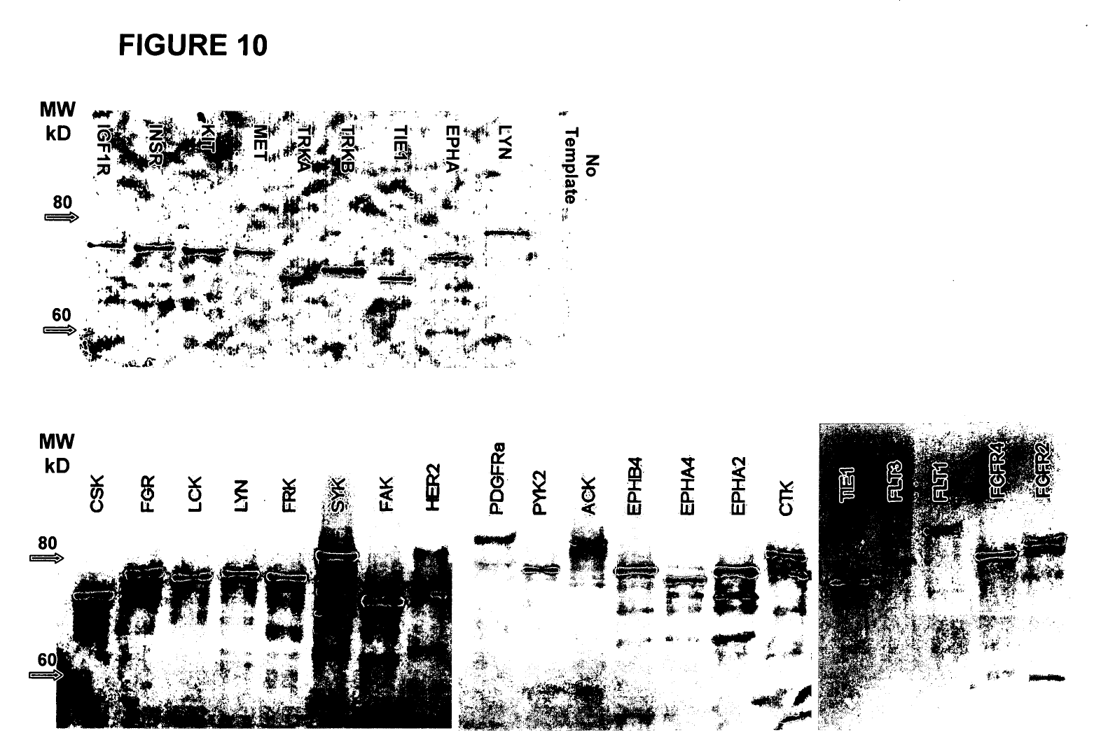

Results of the expression and purification of the GST-IGF1R fusion protein are shown in FIG. 9 and FIG. 10.

6.1.4 Detecting Tyrosine Kinase Activity in ALPHASCREEN™ Assay

Kinase activity measurements were done using ALPHASCREEN™ Phosphotyrosine (PT66) Assay Kit (PerkinElmer) and FUSION microplate plate reader (PerkinElmer) in general agreement with the manufacturer's protocols. Briefly, diluted aliquots of an in vitro translation reaction or purified kinase were mixed with kinase buffer (50 mM Tris-HCl pH 7.5, 5 mM MgCl₂, 5 mM MnCl₂, 2 mM freshly added DTT, 0.01% Tween 20) containing 50 μM ATP and 1 ng/μl of biotinylated polyGlu-Tyr (polyGT-Bio) in a total volume of 15 μl and incubated for 30 min at room temp. After incubation, 10 μl (5+5 μl) of the mixture of donor and acceptor beads were added to the kinase reaction. Both the donor and acceptor beads have to be diluted to 20 ng/ml (50-fold) from the kit stock first in detection buffer (62.5 mM HEPES pH 7.4, 250 mM NaCl, 100 mM EDTA, 0.25% BSA). The mixture was incubated for 60 minutes at ambient temperature (˜25° C.) temperature with moderate shaking and read in PE Fusion Reader using the ALPHASCREEN™ (PerkinElmer) data acquisition software. Translation mix programmed with the empty transcription/translation vector template was used as a negative control in all experiments. Purified recombinant IGF1R kinase produced in SF9 insect cells infected by baculovirus was used as a positive control for tyrosine kinase activity.

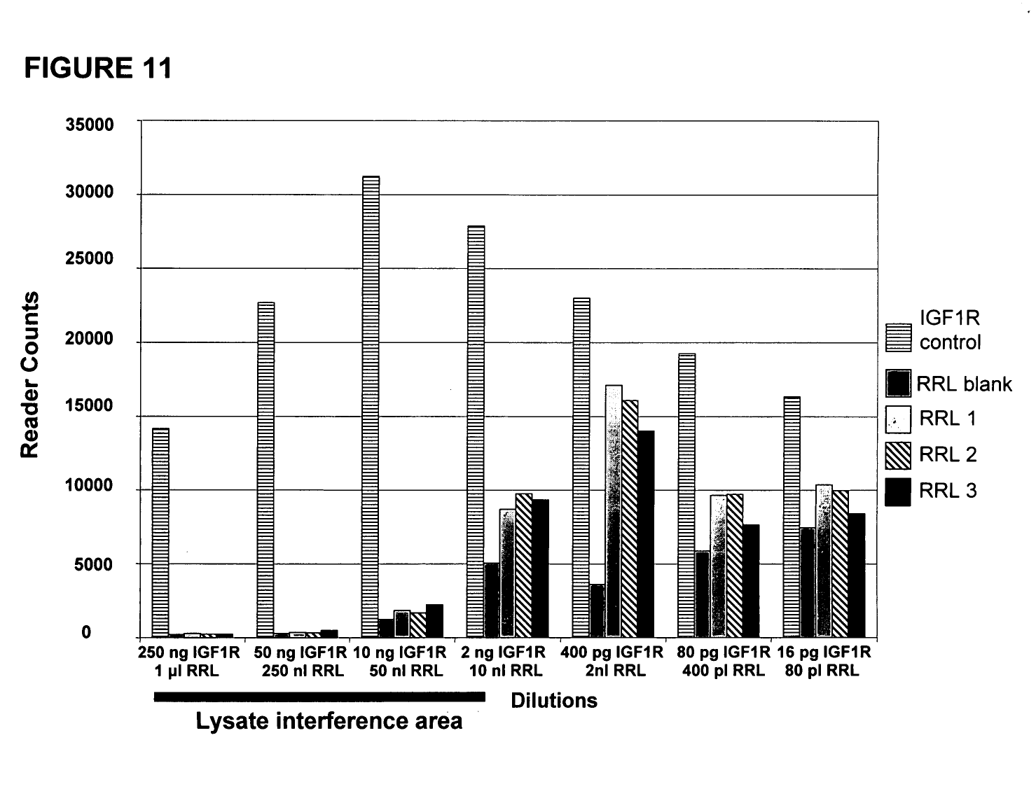

Results of the kinase assays are shown in FIG. 11 (results of three independent experiments). Note that due to the presence of high concentration of optically absorbing material in the reticulocyte lysate, signal in the low dilution area is strongly reduced when crude, nonfractionated translation mix is used directly in the assay.

6.1.5 Detecting Tyrosine Kinase Activity in ELISA Assay

Multiwell plates for the assay were prepared by covering with a generic tyrosine kinase substrate poly(Glu-Tyr), 4:1. A solution of biotinylated Poly(Glu-Tyr) peptide (Upstate Biotechnology, Cat. 12-440) in Hank's Balanced Salt Solution (HBSS) buffer (10 μg/ml) was added at 10-50 μL/well to a commercial streptavidin-coated 96- or 384-well plate (e.g., Neutravidin HBC 96-well, flat-bottom white plates, Pierce Chemical, Cat. 15509), and incubated for 2 hours at room temperature or overnight at 4° C. The plates were then washed three times with 100 μL per well of HBSS and patted dry.

Kinase reactions were run in 75 μL volume per well (for 96-well plate) containing diluted IVT mix programmed with the corresponding kinase DNA, test compound at a desired concentration (e.g., 1 μM), and 100 μM ATP in 1× kinase buffer (20 mM HEPES-K, pH 7.4, 10 mM MgCl₂, 5 mM MnCL₂, 1 mM DTT, 0.1% Triton X-100) with 2 hour incubation at room temperature. IVT reactions were set up as described above (Section 6.1.3) using either SP6 or T7 TNT™ Quick Coupled Transcription/Translation System kits (Promega) and used for the assay immediately after translation without purification, at a typical lysate dilution of 1:50 to 1:250. After incubation, the kinase reaction mix was aspirated from the wells. The plates were washed three times with 100 μL per well of HBSS+0.02% Tween-20 (Wash Buffer), patted dry, and supplied with 50 μL anti-phosphotyrosine antibody 4G10-HRP conjugate (Upstate Biotechnology, Cat. 16-184) diluted 1:30,000 in Starting Block Buffer (Pierce Chemical, Cat. 37538). After incubating for 2 hours at room temperature, plates were washed 4 times with 100 μL per well of Wash Buffer, patted dry, and supplied with 50 per well of SUPERSIGNAL® Pico Substrate (Pierce Chemical, Cat. 37070). After incubating for 5 minutes, the plates were read in luminescence mode on a multiwell plate reader (Alpha Fusion, Perkin-Elmer) and percent inhibition values for each kinase were calculated.

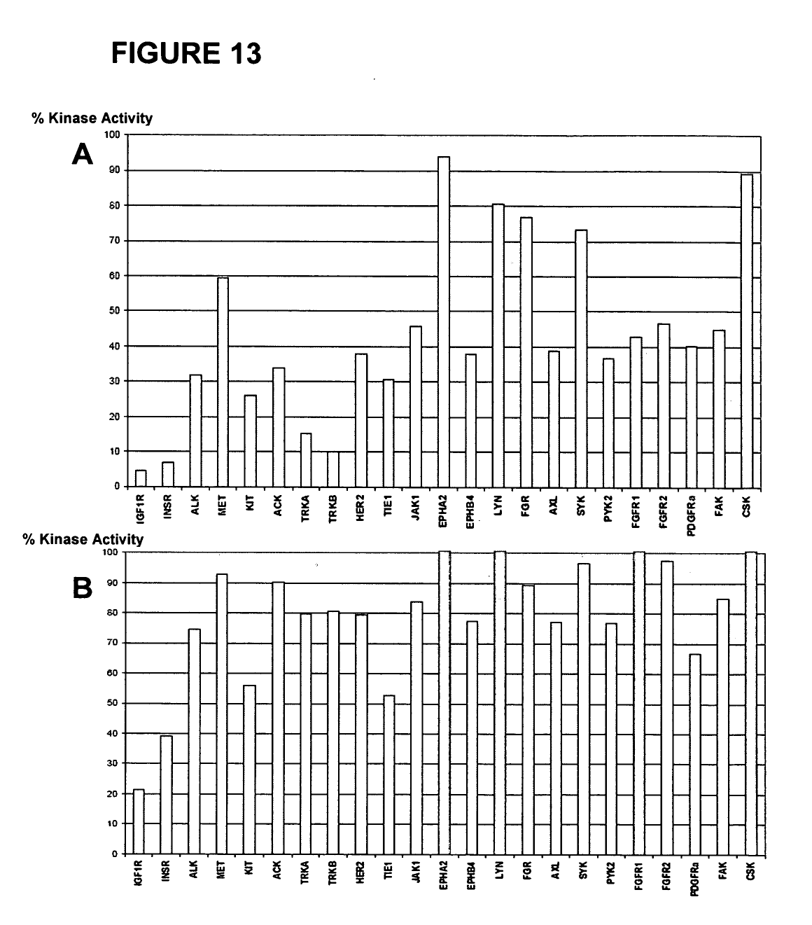

Results of the kinase inhibition profiling of two examplary test compounds, “Compound A” and “Compound B,” which were tested at a concentration of 1 μM are shown in FIG. 13.

6.2 Expression of ABL Tyrosine Kinases

Expression of ABL tyrosine kinase was completed essentially as described in Example 6.1 except that the ABL kinase domain module (FIG. 5R) is inserted into the GST expression cassette in place of the IGF-IR kinase domain module.

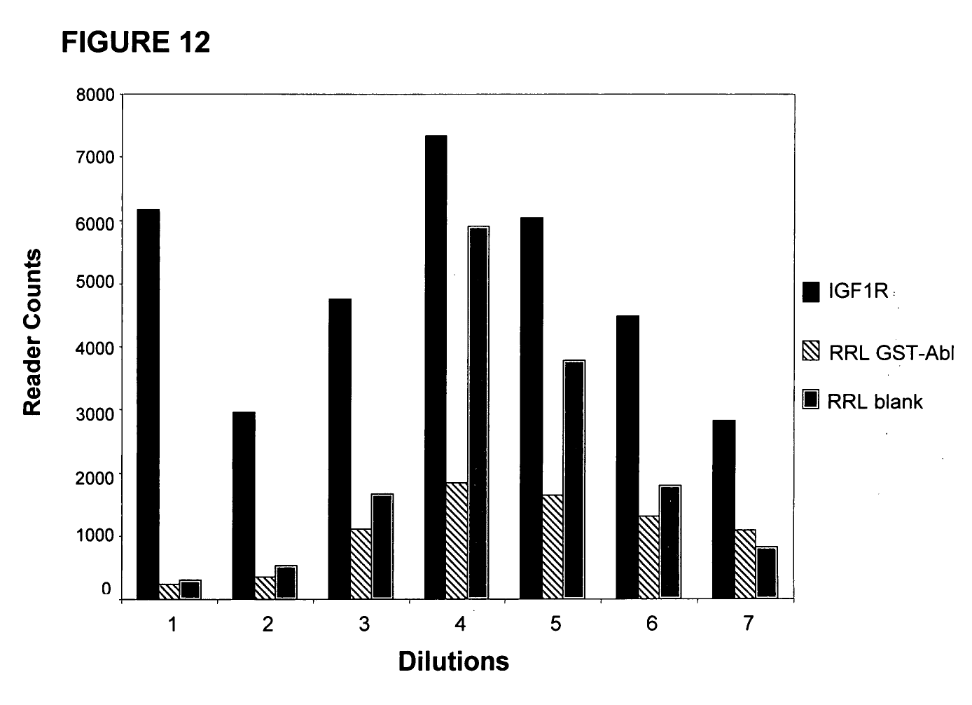

Purification and activity of the kinase was also performed essentially as described in Example 6.1. Results of the kinase assays are shown in FIG. 12.

6.3 Expression of SRC Tyrosine Kinase

Expression, purification and activation assays of SRC tyrosine kinase is completed essentially as described in Example 6.1 except that the SRC kinase domain module (FIG. 5Q) is inserted into the GST expression cassette in place of the IGF-1R kinase domain module. Expression of the tyrosine kinases can be completed simultaneously or sequentially with other PTK as described herein.

6.4 Cloning and Expression of Other Tyrosine Kinases

Expression, purification and activation assays of other tyrosine kinases is completed essentially as described in Example 6.1 except that the kinase domain module is inserted into the GST expression cassette in place of the IGF-1R kinase domain module. Non-limiting examples of kinase domain modules that may be cloned and expressed using the methods provided here (e.g., insertion into the GST expression cassette) are show in FIGS. 5A-5Z and FIG. 6A-6Z. These 52 tyrosine kinase domains (e.g., FIG. 5A-Y represents 16 RTKs and 9 CTKs) correspond to the subset of PTKs representing all major, therapeutically attractive subfamilies of the tyrosine kinase family (see FIGS. 1 and 2). For the RTKs, the complete cytoplasmic tails beginning right after the transmembrane domain to the C-terminus can be chosen. For the CTKs, sequences can vary from, for example, fill-length for smaller kinases like SRC and BRK to fragments including a kinase domain, and, for example, 200-300 amino acids in flanking sequences.

Cloning of additional PTK (including mutant forms) is within the skill of those in the art and can also be similarly inserted into the GST expression cassette. Expression of the tyrosine kinases can be completed simultaneously or sequentially with other PTK as described herein.

1. A method for producing a panel of protein tyrosine kinases (PTK) and/or fragments thereof, wherein substantially all of said PTK in the panel have kinase activity, said method comprising:
providing one or more polynucleotides that encode one or more polypeptides comprising a tyrosine kinase domain and/or fragment thereof,
optionally adding a first tag to the N-terminus of one or more of the polynucleotides,
optionally adding a second tag to the C-terminus of one or more of the polynucleotides,
translating the one or more polynucleotides in an in vitro cell-free translation (IVT) system, wherein the resulting one or more polypeptides comprise a tyrosine kinase domain, and/or fragment thereof, having kinase activity.

2. A method of screening for a modulator of tyrosine kinase activity, comprising:
providing one or more polynucleotides that encode one or more polypeptides comprising a tyrosine kinase domain and/or fragment thereof,
optionally adding a first tag to the N-terminus of one or more of the polynucleotides,
optionally adding a second tag to the C-terminus of one or more of the polynucleotides,
translating the one or more polynucleotides in an in vitro cell-free translation (IVT) system, wherein the resulting one or more polypeptides comprise a tyrosine kinase domain, and/or fragment thereof, having kinase activity,
contacting a test compound with the one or more polypeptides, and
detecting modulation of kinase activity the one or more polypeptides relative to kinase activity in the absence of test compound.

3. A method for modulating tyrosine kinase activity in a patient, comprising:
providing one or more polynucleotides that encode one or more polypeptides comprising a tyrosine kinase domain and/or fragment thereof,
optionally adding a first tag to the N-terminus of the one or more polynucleotides,
optionally adding a second tag to the C-terminus of the one or more polynucleotides,
translating the one or more polynucleotides in an in vitro cell-free translation (IVT) system, wherein the resulting one or more polypeptides comprise a tyrosine kinase domain, and/or fragment thereof, having kinase activity,
contacting a test compound with the one or more polypeptides,
detecting modulation of kinase activity of the one or more polypeptides relative to kinase activity in the absence of test compound, and
administering the test compound to the patient, wherein the test compound modulates kinase activity in the patient relative to kinase activity in the absence of test compound.

4. The method of claim 1, wherein about 5, 10, 15, 20, 30, 40, 50, 60, 70, 80, 90, 100, 150, 200, 250, 300 350, 400, 450, 500, 550, 600, 650, 700, 750, 800, 850, 900, 950, 1000 or more polynucleotides are provided.

5. The method of claim 1, wherein the one or more polynucleotides encode about 5, 10, 15, 20, 30, 40, 50, 60, 70, 80, 90, 100, 150, 200, 250, 300 350, 400, 450, 500, 550, 600, 650, 700, 750, 800, 850, 900, 950, 1000 or more polypeptides.

6. The method of claim 1, wherein the tyrosine kinase domain or fragment thereof is a non-receptor tyrosine kinase.

7. The method of claim 1, wherein the tyrosine kinase domain or fragment thereof is a receptor tyrosine kinase.

8. The method of claim 1, wherein the tyrosine kinase domain or fragment thereof is a combination of one or more non-receptor tyrosine kinases and one or more receptor tyrosine kinases.

9. The method of claim 1, wherein the tyrosine kinase domain or fragment thereof is selected from EGFR, IGF1R, KIT, VEGFR1, FGFR1, TRKA, MET, EPHB4, AXL, TIE1, DDR1, RET, ROS, ALK, ROR1, MUSK, SRC, ABL, JAK1, ACK1, FAK, FES, BRK, TEC, ZAP70, BLK, BMX, BTK, CSFR, CSK, CTK, DDR2, EPHA2, EPHA4, FGFR2, FGFR4, FGR, FLT3, FRK, FYN, HER2, HER3, JAK2, JAK3, KDR, LCK, LYN, PDGFRα, PYK2, SYK, TIE2, YES and combinations thereof.

10. The method of claim 1, wherein the one or more polypeptides comprising the tyrosine kinase domain or fragment thereof are one or more polypeptides comprising different tyrosine kinase domains, fragments of different tyrosine kinase domains, or combinations thereof.

11. The method of claim 1, wherein the one or more polypeptides comprising the tyrosine kinase domain or fragment thereof are one or more polypeptides comprising the same tyrosine kinase domain, fragments of the same tyrosine kinase domain, or combinations thereof.

12. The method of claim 1, wherein the one or more polypeptides comprising the tyrosine kinase domain or fragment thereof are one or more polypeptides comprising the same tyrosine kinase domain, a fragment of the same tyrosine kinase domain, different tyrosine kinase domains, a fragment of different tyrosine kinase domains, or combinations thereof.

13. The method of claim 1, wherein the one or more polypeptides comprising the tyrosine kinase domain or fragment thereof are one or more polypeptides comprising tyrosine kinases or fragments thereof from different families, different subfamilies, the same family, the same subfamily, or combinations thereof.

14. The method of claim 1, wherein the one or more polypeptides comprising the tyrosine kinase domain or fragment thereof are in the same form of the same kinase domain, the same form of different kinase domains, different forms of the same kinase domain, different forms of different kinase domains, or combinations thereof.

15. The method of claim 14, wherein the form is a wild-type form of a tyrosine kinase domain, a mutant form of a tyrosine kinase domain, or a combination thereof.

16. The method of claim 14, wherein the form is a mutant form of a tyrosine kinase domain.

17. The method of claim 1, wherein the one or more polynucleotides are linear polynucleotides.

18. The method of claim 1, wherein the in vitro cell-free translation system is a wheat germ extract (WGE), rabbit reticulocyte lysate (RRL), E. coli S30 (S30) cell-free translation system, or a combination thereof.

19. The method of claim 1, wherein the first tag is a detectable tag.

20. The method according to claim 19, wherein the detectable tag is a fluorescent tag.

21. The method according to claim 20, wherein the fluorescent tag is Cys-Cys-Pro-Gly-Cys-Cys (SEQ ID NO:54).

22. The method according to claim 19, wherein the second tag is an affinity tag.

23. The method of claim 1, wherein the second tag is an affinity tag.

24. The method according to claim 23 wherein the second tag is Trp-Ser-His-Pro-Gln-Phe-Gly-Lys (SEQ ID NO:55).

25. The method according to claim 23, wherein the affinity tag is a dimerization domain.

26. The method according to claim 24, wherein the dimerization domain is glutathione-S-transferase (GST).

27. The method of claim 1, wherein the second tag is a detectable tag.

28. The method according to claim 27, wherein the first tag is an affinity tag.

29. The method of claim 1, wherein the first tag is an affinity tag.

30. The method according to claim 29, wherein the second tag is a detectable tag.

31. The method of claim 1, wherein the tyrosine kinase domain or fragment thereof is a human non-receptor tyrosine kinase or fragment thereof.

32. The method of claim 31, wherein the human non-receptor tyrosine kinase or fragment thereof is selected from the group consisting of a member of the ABL family, a member of the ACK family, a member of the CSK family, a member of the FAK family, a member of the FES family, a member of the FRK family, a member of the JAK family, a member of the SRC-A family, a member of the SRC-B family, a member of the TEC family, a member of the SYK family, and combinations thereof.

33. The method of claim 1, wherein the tyrosine kinase domain or fragment thereof is a human receptor tyrosine kinase or fragment thereof.

34. The method of claim 33, wherein the human receptor tyrosine kinase or fragment thereof is selected from the group consisting of a member of the ALK family, a member of the AXL family, MER, TYRO3, a member of the DDR family, a member of the EGFR family, ERBB2, ERBB3, ERBB4, a member of the EPH family, a member of the FGFR family, a member of the INSR family, a member of the MET family, a member of the MUSK family, a member of the PDGFR family, a member of the PTK7 family, a member of the RET family, a member of the ROR family, a member of the ROS family, a member of the RYK family, a member of the TIE family, a member of the TRK family, a member of the VEGFR family, a member of the AATYK family, a member of the SuRTK106 family, and combinations thereof.

35.-48. (canceled)

49. A method of treating a protein tyrosine kinase-associated disorder or disease state in a patient comprising administering to the patient the test compound identified by the method of claim 3, thereby alleviating a symptom of the protein kinase-associated disorder or disease state.

50.-54. (canceled)

55. A method of preventing a protein tyrosine kinase-associated disorder or disease state in a patient comprising admninistering to the patient the test compound identified by the method of claim 3, thereby preventing a symptom of the protein kinase-associated disorder or disease state.

56. A kit for screening for a modulator of tyrosine kinase activity comprising:
one or more polynucleotides that encode for one or more polypeptides comprising a tyrosine kinase domain and/or fragment thereof, wherein said polynucleotides optionally further comprises a first tag on the N-terminus of the one or more polynucleotide and/or a second tag on the C-terminus of the one or more polynucleotides, and
an in vitro translation system.
